(19) United States
(12) Patent Application Publication
(10) Pub. No.: US 2013/0190248 A1
(43) Pub. Date: Jul. 25, 2013

(54) SUBSTITUTED PEPTIDE ANALOGS

(71) Applicants: Brian Alan MENDELSOHN, Newcastle, WA (US); Stuart Daniel BARNSCHER, Langley (CA)

(72) Inventors: Brian Alan MENDELSOHN, Newcastle, WA (US); Stuart Daniel BARNSCHER, Langley (CA)

(73) Assignee: AGENSYS, INC., Santa Monica, CA (US)

(21) Appl. No.: 13/557,044

(22) Filed: Jul. 24, 2012

Related U.S. Application Data
(60) Provisional application No. 61/511,943, filed on Jul. 26, 2011.

(51) Int. Cl. C07K 5/02

(52) U.S. Cl. CPC C07K 5/02
USPC 514/19.3; 530/332

(57) ABSTRACT

The present invention relates to peptide analogs, antibody-drug conjugates comprising such compounds, pharmaceutical compositions comprising such compounds and conjugates, and methods of treating cancer with such compounds and conjugates.

### CROSS-REFERENCE TO RELATED APPLICATIONS

This application is a non-provisional patent application, which claims the benefit of priority from U.S. Provisional Patent Application No. 61/511,943, filed Jul. 26, 2011. The contents of each application listed in this paragraph are fully incorporated by reference herein.

### FIELD OF THE INVENTION

The present invention relates to peptide analogs, antibody-drug conjugates comprising such compounds, pharmaceutical compositions comprising such compounds and conjugates, and methods of treating cancer with such compounds and conjugates.

### BACKGROUND OF THE INVENTION

Cancer is the second leading cause of human death exceeded only by coronary disease. In the U.S., cancer accounts for nearly 1 in 4 deaths. Worldwide, millions of people die from cancer every year. In the United States alone, as reported by the American Cancer Society, cancer causes the death of well over a half-million people annually, with over 1.5 million new cases diagnosed per year. While deaths from heart disease have been declining significantly, those resulting from cancer generally are on the rise. In the early part of the next century, cancer is predicted to become the leading cause of death unless new medicines are found.

Worldwide, several cancers stand out as the leading killers. In particular, carcinomas of the lung, prostate, breast, colon, pancreas, ovary, and bladder represent the primary causes of cancer death. With very few exceptions, metastatic cancer is fatal. Moreover, even for those cancer patients who initially survive their primary cancers, common experience has shown that their lives are dramatically altered. Many cancer patients experience strong anxieties driven by the awareness of the potential for recurrence or treatment failure. Many cancer patients experience physical debilitations following treatment. Furthermore, many cancer patients experience a recurrence.

Promising new cancer therapeutics include the dolastatins and synthetic dolastatin analogs such as auristatins (U.S. Pat. Nos. 5,635,483, 5,780,588, 6,323,315, and 6,884,869; Shnyder et al. (2007) Int. J. Oncol. 31:353-360; Otani, M. et al. Jpn. J. Cancer Res. 2000, 91, 837-844; PCT Intl. Publ. Nos. WO 01/18032 A3, WO 2005/039492, WO2006/132670, and WO 2009/095447; Fennell, B. J. et al. J. Antimicrob. Chemther. 2003, 51, 833-841). Dolastatins and auristatins have been shown to interfere with microtubule dynamics, thus disrupt cell division (Woyke et al. (2001) Antimicrob. Agents Chemother. 45(12):3580-3584) and have anticancer (U.S. Pat. No. 5,663,149) and antifungal activity (Pettit et al. (1998) Antimicrob. Agents Chemother. 42:2961-2965). Unfortunately, despite early enthusiasm, dolastatin 10 showed poor results as a single agent in phase II clinical trials (Shnyder (2007), supra). Certain compounds in the auristatins family have shown greater promise as clinical candidates with improved efficacy and pharmacological characteristics over the dolastatins (Pettit et al. (1995) Anti-Cancer Drug Des. 10:529-544; Pettit et al. (1998) Anti-Cancer Drug Des. 13:243-277; Shnyder (2007), supra). Various synthetic analogs of this structural type have been described (U.S. Pat. No. 6,569,834; U.S. Pat. No. 6,124,431; and Pettit et al. (2011) J. Nat. Prod. 74:962-968).

The auristatins have several properties which make them attractive for pharmaceutical development. First, these compounds are extremely potent. Second, their preparation is straight-forward because of the peptidic scaffold. Third, they possess good pharmacokinetic and metabolic profiles compared to peptides in general, or to other cancer drug classes in particular. Finally, the peptidic structure of the auristatins is similar to that of an antibody, so when these compounds are used as part of an antibody-drug conjugate (ADC), they are less likely to cause precipitation or formation of high molecular weight aggregates (Doronina et al. (2003) Nat. Biotechnology 21(7):778-784).

The use of ADC's for the delivery of cytotoxic or cytostatic agents, i.e. drugs to kill or inhibit tumor cells in the treatment of cancer (Syrigos and Epenetos (1999) Anticancer Res. 19:605-614; Niculescu-Duvaz and Springer (1997) Adv. Drug Delivery Rev. 26:151-172; U.S. Pat. No. 4,975,278) allows targeted delivery of the drug moiety directly to tumors. In contrast, systemic administration of these unconjugated drug agents may result in unacceptable levels of toxicity as they will affect normal cells as well as tumor cells (Baldwin et al. (1986) Lancet (Mar. 15, 1986):603-05; Thorpe (1985) “Antibody Carriers of Cytotoxic Agents in Cancer Therapy: A Review,” in Monoclonal Antibodies '84: Biological and Clinical Applications, A. Pinchera et al. (eds.), pp. 475-506). Cytotoxic and cytostatic toxins have several mechanisms of action including tubulin binding, DNA binding, or topoisomerase inhibition. Toxins used in ADC's include radioisotopes, bacterial toxins, plant toxins, and small molecule toxins such as geldanamycin (Mandler et al. (2000) J. Natl. Cancer Inst. 92(19):1573-1581; Mandler et al. (2000) Bioorg. Med. Chem. Lett. 10:1025-1028; Mandler et al. (2002) Bioconjugate Chem. 13:786-791), maytansinoids (EP 1391213; Liu et al. (1996) Proc. Natl. Acad. Sci. USA 93:8618-8623), calicheamicin (Lode et al. (1998) Cancer Res. 58:2928; Hinman et al. (1993) Cancer Res. 53:3336-3342), daunomycin, doxorubicin, methotrexate, and vindesine (Rowland et al. (1986), supra). Not all cytotoxic drugs are suited for ADC use because they are inactive or less active when conjugated to proteins such as antibodies.

Several ADC's have shown promising results against cancer in clinical trials, including: 1) ZEVALIN® (ibritumomab tiuxetan, Biogen/Idec), an antibody-radioisotope conjugate composed of a murine IgG₁ kappa monoclonal antibody directed against the CD20 antigen found on the surface of normal and malignant B lymphocytes and ¹¹¹In or ⁹⁰Y radioisotope bound by a thiourea linker-chelator; 2) MYLOTARG™ (gemtuzumab ozogamicin, Wyeth Pharmaceuticals), an antibody drug conjugate composed of a hu CD33 antibody linked to calicheamicin, which was approved in 2000 for the treatment of acute myeloid leukemia by injection, but discontinued in 2010 due to lack of efficacy; 3) cantuzumab mertansine (Immunogen, Inc.), an antibody drug conjugate composed of the hu C242 antibody linked via the disulfide linker SPP to the maytansinoid drug moiety, DM1, which is advancing in human trials for the treatment of cancers that express CanAg, such as colon, pancreatic, gastric, and others; and MLN-2704 (Millennium Pharm., BZL Biologics, Immunogen Inc.), an antibody drug conjugate composed of the anti-prostate specific membrane antigen (PSMA) monoclonal antibody linked to the maytansinoid drug moiety, DM1, which is under development for the potential treatment of prostate tumors.

The auristatin peptides, auristatin E (AE), monomethylauristatin F, and monomethylauristatin E (MMAE), synthetic analogs of dolastatin 10, also show promise. These have been conjugated to chimeric monoclonal antibodies cBR96 (specific to the Lewis Y carbohydrate antigen on carcinomas) and cAC10 (specific to CD30 on hematological malignancies) (Doronina et al. (2003), supra). ADCETRIS™ has been clinically approved and several others are under therapeutic development. Antibody-drug conjugates of synthetic analogs of the dolastatins were described in U.S. Pat. No. 7,498,298.

Despite significant advances, there remains a need for new anticancer therapeutics, including small molecule drugs and ADC's, with desirable pharmaceutical properties.

### SUMMARY OF THE INVENTION

In one aspect, the present invention provides for novel peptide analogs. Thus, the invention relates to a compound of Formula (I):

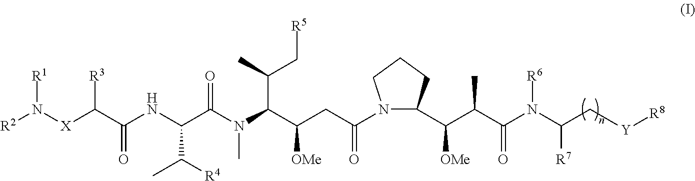

wherein
R¹ and R² are each independently H, methyl, or OH; or R¹ and R² taken together with the N to which they are attached form a 3- to 8-membered heterocyclic ring, optionally substituted with C₁₋₄alkyl, hydroxy, or C₁₋₄alkoxy;
X is O or is absent;
R³ is an amino acid substituent;
   wherein R³ is in the R or S stereochemical configuration relative to the remainder of the molecule;
   or, alternatively, the —(CH(R³))— group is a phenyl;
R⁴ is methyl or ethyl;
R⁵ is H or methyl;
R⁶ is H or C₁₋₈alkyl;
R⁷ is H, C₁₋₈alkyl, —CO₂R^{a}, —(OCH₂CH₂)ₘ—OR^{b}, —NH—CH₂CH₂—N(CH₃)₂, —B(OR^{c})₂, or tetrazole;
   wherein R^{a}, R^{b}, and R^{c} are each independently H or C₁₋₄alkyl;
   m is an integer from 1 to 12; and
R⁷ is in the R or S stereochemical configuration relative to the remainder of the molecule;
n is 0, 1, 2, 3, or 4;
Y is O or is absent; and
R⁸ is (1) or (2):
   (1) —NR^{d}R^{e} wherein R^{d} and R^{e} are each independently H, C₁₋₄alkyl, OH, SH, —NR^{f}R^{g}, —NHCOR^{g}, halo, —COR^{h}, or a phenyl or 3- to 8-membered heterocyclic ring, each optionally substituted with C₁₋₄alkyl, hydroxy, or C₁₋₄alkoxy;
      wherein R^{f} and R^{g} are each independently H, C₁₋₄alkyl, phenyl, or a 3- to 8-membered heterocyclic ring;
      R^{h} is H, —OH, —OC₁₋₄alkyl, —NRⁱR^{j}, —NHNH₂, or —ONH₂; and
         Rⁱ and R^{j} are each independently H, C₁₋₄alkyl, phenyl, or a 3- to 8-membered heterocyclic ring;
   or R^{d} and R^{e} taken together with the nitrogen to which they are attached form a 5- to 8-membered heterocyclic ring, optionally substituted with one or two substituents selected from the group consisting of C₁₋₄alkyl, halo, OH, —OC₁₋₄alkyl, SH, —NR^{k}R^{l}, —NHC(O)R^{l}, —NHCO₂R^{l}, cyano, —CONR^{k}R^{l}, —COR^{m}, phenyl, and a 3- to 8-membered heterocyclic ring;
      where R^{k} and R^{l} are each independently H, C₁₋₄alkyl, phenyl, or a 3- to 8-membered heterocyclic ring; or
      R^{k} and R^{l} taken together with the nitrogen to which they are attached form a 3- to 8-membered heterocyclic ring; and
      R^{m} is H, OH, —OC₁₋₄alkyl, —NHNH₂, or —ONH₂;
   (2) a phenyl or a carbon-linked, nitrogen-containing heteroaryl ring, each optionally substituted with one, two, or three substituents selected from the group consisting of halo, OH, SH, —NRⁿR^{o}, —NHCOR^{o}, —COR^{p}, phenyl, anilino, and a 3- to 8-membered heterocyclic ring;
      wherein Rⁿ and R^{o} are each independently H, C₁₋₄alkyl, phenyl, or a 3- to 8-membered heterocyclic ring;
      R^{p} is H, OH, —NR^{q}R^{r}, —NHNH₂, or —ONH₂; and
         Wherein R^{q} and R^{r} are each independently H, C₁₋₄alkyl, phenyl, or a 3- to 8-membered heterocyclic ring;
   wherein n is not 0 when R⁸ is —NR^{d}R^{e};
with the proviso that when R¹ and R² are both methyl,

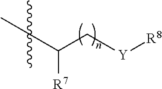

is not 2-(2-pyridyl)ethyl, phenethyl, 4-hydroxyphenethyl, 2-morpholinoethyl, 3-dimethylaminopropyl, 2-dimethylaminoethyl, 4-aminophenethyl, 2-(5-methoxy-1H-indol-2-yl)ethyl, or 1-methoxy-1-oxo-3-phenylpropan-2-yl;
or a pharmaceutically acceptable salt thereof.

The present invention further contemplates antibody-drug conjugates comprising the novel peptide analogs described herein covalently bound to an antibody. Thus, in a further aspect, the present invention relates to an antibody-drug conjugate of Formula (II):

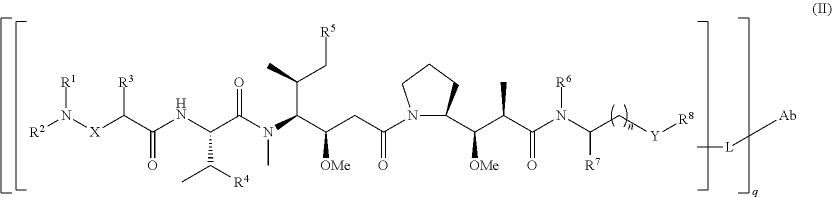

wherein R¹, R², R³, R⁴, R⁵, R⁶, R⁷, R⁸, X, Y, and n are defined as for Formula (I);
L is a linker;
q is an integer from 1 to 20; and
Ab is an antibody;
wherein each -L- unit is covalently bound to Ab and to the

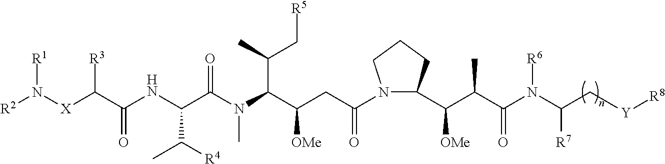

structure.

In a further aspect, the invention relates to a pharmaceutical composition comprising an effective amount of at least one compound of Formula (I), or a pharmaceutically acceptable salt thereof. In a further aspect, the invention relates to a pharmaceutical composition comprising an effective amount of at least one antibody-linker-drug conjugate of Formula (II).

In yet another aspect, the invention relates to a method of treating a subject suffering from or diagnosed with cancer, comprising administering to a subject in need of such treatment an effective amount of at least one compound of Formula (I), or a pharmaceutically acceptable salt thereof. In a further aspect, the invention relates to a method of treating a subject suffering from or diagnosed with cancer, comprising administering to a subject in need of such treatment an effective amount of at least one antibody-linker-drug conjugate of Formula (II).

### BRIEF DESCRIPTION OF THE FIGURES

FIG. 1 shows the results for Example 1 in an in vitro cytotoxicity experiment using T47D cells, as described in Example 23. Data is graphed as percent survival versus concentration of test compound, compared to untreated control wells.

FIG. 2 shows the results for Examples 1 and 2 in an in vitro cytotoxicity experiment using SW780 cells, as described in Example 23. Data is graphed as percent survival versus concentration of test compound, compared to untreated control wells.

FIG. 3 shows the results for Examples 1, 2, and 21 in an in vitro cytotoxicity experiment using HCC-1954 cells, as described in Example 23. Data is graphed as percent survival versus concentration of test compound, compared to untreated control wells.

FIG. 4 shows the results for Examples 1, 2, and 21 in an in vitro cytotoxicity experiment using PC3 cells, as described in Example 23. Data is graphed as percent survival versus concentration of test compound, compared to untreated control wells.

FIG. 5 shows the results for Examples 1, 2, and 21 in an in vitro cytotoxicity experiment using SKOV3 cells, as described in Example 23. Data is graphed as percent survival versus concentration of test compound, compared to untreated control wells.

FIG. 6 shows the results for Examples 1, 2, and 21 in an in vitro cytotoxicity experiment using HCT15 cells, as described in Example 23. Data is graphed as percent survival versus concentration of test compound, compared to untreated control wells.

FIG. 7 shows the results for Examples 3, 4, and 5 in an in vitro cytotoxicity experiment using HCC-1954 cells, as described in Example 23. Data is graphed as percent survival versus concentration of test compound.

FIG. 8 shows the results for Examples 3, 4, and 5 in an in vitro cytotoxicity experiment using PC3 cells, as described in Example 23. Data is graphed as percent survival versus concentration of test compound.

FIG. 9 shows the results for Examples 3, 4, and 5 in an in vitro cytotoxicity experiment using SKOV3 cells, as described in Example 23. Data is graphed as percent survival versus concentration of test compound.

FIG. 10 shows the results for Examples 3, 4, and 5 in an in vitro cytotoxicity experiment using T47D cells, as described in Example 23. Data is graphed as percent survival versus concentration of test compound.

FIG. 11 shows the results for Examples 6, 7, and 22 in an in vitro cytotoxicity experiment using HCC-1954 cells, as described in Example 23. Data is graphed as percent survival versus concentration of test compound.

FIG. 12 shows the results for Examples 6, 7, and 22 in an in vitro cytotoxicity experiment using PC3 cells, as described in Example 23. Data is graphed as percent survival versus concentration of test compound.

FIG. 13 shows the results for Examples 6, 7, and 22 in an in vitro cytotoxicity experiment using SKOV3 cells, as described in Example 23. Data is graphed as percent survival versus concentration of test compound.

FIG. 14 shows the results for Examples 6, 7, and 22 in an in vitro cytotoxicity experiment using T47D cells, as described in Example 23. Data is graphed as percent survival versus concentration of test compound.

FIG. 15 shows the results for Examples 9, 10, 14, and 20 in an in vitro cytotoxicity experiment using HCC-1954 cells, as described in Example 23. Data is graphed as percent survival versus concentration of test compound.

FIG. 16 shows the results for Examples 9, 10, 14, and 20 in an in vitro cytotoxicity experiment using PC3 cells, as described in Example 23. Data is graphed as percent survival versus concentration of test compound.

FIG. 17 shows the results for Examples 9, 10, 14, and 20 in an in vitro cytotoxicity experiment using SKOV3 cells, as described in Example 23. Data is graphed as percent survival versus concentration of test compound.

FIG. 18 shows the results for Examples 9, 10, 14, and 20 in an in vitro cytotoxicity experiment using HCT15 cells, as described in Example 23. Data is graphed as percent survival versus concentration of test compound.

FIG. 19 shows the results for Examples 8, 12, and 17 in an in vitro cytotoxicity experiment using HCC-1954 cells, as described in Example 23. Data is graphed as percent survival versus concentration of test compound.

FIG. 20 shows the results for Examples 8, 12, and 17 in an in vitro cytotoxicity experiment using PC3 cells, as described in Example 23. Data is graphed as percent survival versus concentration of test compound.

FIG. 21 shows the results for Examples 8, 12, and 17 in an in vitro cytotoxicity experiment using SKOV3 cells, as described in Example 23. Data is graphed as percent survival versus concentration of test compound.

FIG. 22 shows the results for Examples 8, 12, and 17 in an in vitro cytotoxicity experiment using HCT15 cells, as described in Example 23. Data is graphed as percent survival versus concentration of test compound.

FIG. 23 shows the results for Examples 13, 15, 16, and 19 in an in vitro cytotoxicity experiment using HCC-1954 cells, as described in Example 23. Data is graphed as percent survival versus concentration of test compound.

FIG. 24 shows the results for Examples 13, 15, 16, and 19 in an in vitro cytotoxicity experiment using PC3 cells, as described in Example 23. Data is graphed as percent survival versus concentration of test compound.

FIG. 25 shows the results for Examples 13, 15, 16, and 19 in an in vitro cytotoxicity experiment using SKOV3 cells, as described in Example 23. Data is graphed as percent survival versus concentration of test compound.

FIG. 26 shows the results for Examples 13, 15, 16, and 19 in an in vitro cytotoxicity experiment using HCT15 cells, as described in Example 23. Data is graphed as percent survival versus concentration of test compound.

FIG. 27 shows in vitro tubulin polymerization data for tubulin treated with Example 1 and Example 3, as described in Example 24. Untreated (buffer) tubulin shows the basal level of tubulin polymerization. A tubulin stabilizer (Paclitaxel) and a tubulin de-stabilizer (Vinblastine) were used as controls. All compounds were used at a final concentration of 10 μM.

FIG. 28 shows in vitro tubulin polymerization data for tubulin treated with Example 2, as described in Example 24. Untreated (buffer) tubulin shows the basal level of tubulin polymerization. A tubulin stabilizer (Paclitaxel) and a tubulin de-stabilizer (Vinblastine) were used as controls. All compounds were used at a final concentration of 10 μM.

FIG. 29 shows in vitro tubulin polymerization data for tubulin treated with Example 4, Example 7, and Example 22, as described in Example 24. Untreated (buffer) tubulin shows the basal level of tubulin polymerization. A tubulin stabilizer (Paclitaxel) was used as a control. All compounds were used at a final concentration of 10 μM.

FIG. 30 shows in vitro tubulin polymerization data for tubulin treated with Example 1, Example 5, and Example 6, as described in Example 24. Untreated (buffer) tubulin shows the basal level of tubulin polymerization. A tubulin stabilizer (Paclitaxel) was used as a control. All compounds were used at a final concentration of 10 μM.

FIG. 31 shows in vitro tubulin polymerization data for tubulin treated with Example 9, Example 10, Example 11, and Example 19, as described in Example 24. Untreated (buffer) and 0.5% DMSO treated tubulin show the basal level of tubulin polymerization. A tubulin stabilizer (Paclitaxel) was used as a control. All compounds were used at a final concentration of 10 μM.

FIG. 32 shows in vitro tubulin polymerization data for tubulin treated with Example 8, Example 13, Example 17, and Example 20, as described in Example 24. Untreated (buffer) and 0.5% DMSO treated tubulin show the basal level of tubulin polymerization. A tubulin stabilizer (Paclitaxel) was used as a control. All compounds were used at a final concentration of 10 μM.

FIG. 33 shows in vitro tubulin polymerization data for tubulin treated with Example 12, Example 14, Example 15, and Example 16, as described in Example 24. Untreated (buffer) and 0.5% DMSO treated tubulin show the basal level of tubulin polymerization. A tubulin stabilizer (Paclitaxel) was used as a control. All compounds were used at a final concentration of 10 μM. For the sake of brevity, the disclosures of the publications cited in this specification, including patents, are herein incorporated by reference.

FIG. 34 shows the in vivo efficacy of Examples 1, 4, 5, and 6 at two dose levels, as described in Example 25. Results are presented as mean tumor volume versus days of treatment.

FIG. 35 shows the in vivo efficacy of Examples 1, 2, 7, 11, 13, 15, 16, 19, and 22, as described in Example 25. Results are presented as mean tumor volume versus days of treatment.

### DETAILED DESCRIPTION OF THE INVENTION

For the sake of brevity, the disclosures of the publications cited in this specification, including patents, are herein incorporated by reference.

As used herein, the terms “including,” “containing,” and “comprising” are used in their open, non-limiting sense.

To provide a more concise description, some of the quantitative expressions given herein are not qualified with the term “about”. It is understood that, whether the term “about” is used explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value.

The term “alkyl” refers to a straight- or branched-chain alkyl group having from 1 to 12 carbon atoms in the chain. Examples of alkyl groups include methyl (Me), ethyl (Et), n-propyl, isopropyl, butyl, isobutyl, sec-butyl, tert-butyl (tBu), pentyl, isopentyl, tert-pentyl, hexyl, isohexyl, and groups that, in light of the ordinary skill in the art and the teachings provided herein, would be considered equivalent to any one of the foregoing examples.

The term “heterocycloalkyl” refers to a monocyclic, or fused, bridged, or spiro polycyclic ring structure that is saturated or partially saturated and has from 3 to 12 ring atoms per ring structure selected from carbon atoms and up to three heteroatoms selected from nitrogen, oxygen, and sulfur. The ring structure may optionally contain up to two oxo groups on carbon or sulfur ring members. Illustrative entities, in the form of properly bonded moieties, include:

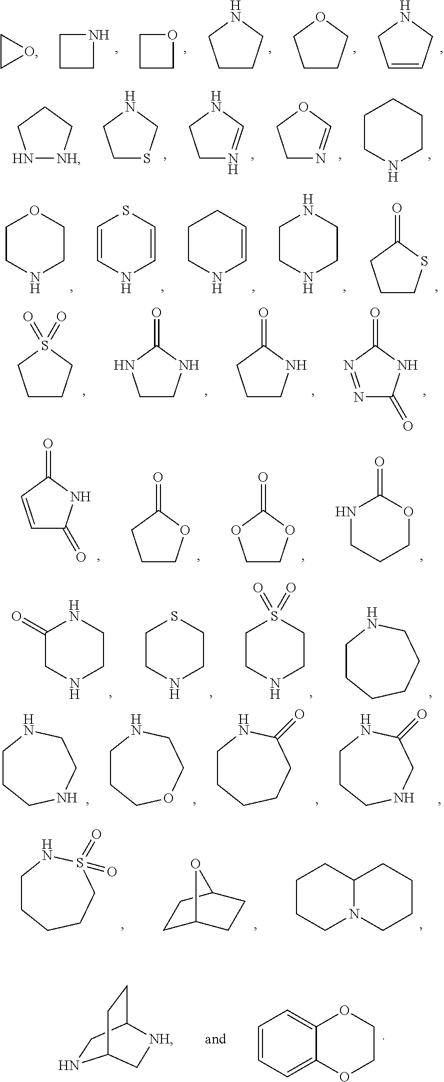

The term “heteroaryl” refers to a monocyclic, fused bicyclic, or fused polycyclic aromatic heterocycle (ring structure having ring atoms selected from carbon atoms and up to four heteroatoms selected from nitrogen, oxygen, and sulfur) having from 3 to 12 ring atoms per heterocycle. Illustrative examples of heteroaryl groups include the following entities, in the form of properly bonded moieties:

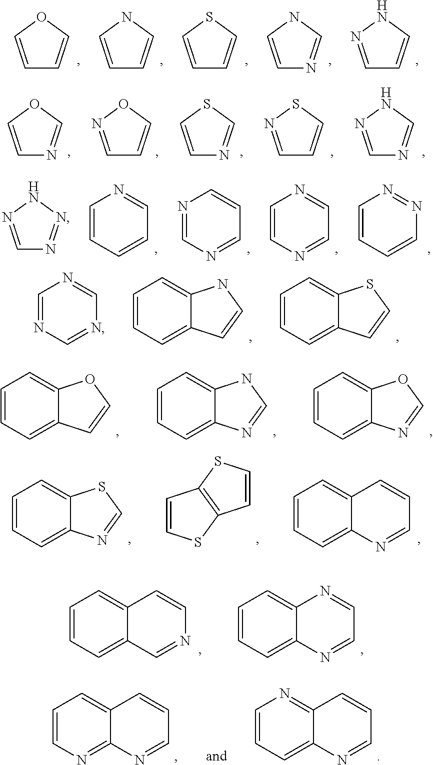

The terms “heterocycle,” “heterocyclic,” or “heterocyclyl” as used herein encompass both the “heterocycloalkyl” and “heteroaryl” moieties as defined above.

Those skilled in the art will recognize that the species of heterocyclyl, heteroaryl and heterocycloalkyl groups listed or illustrated above are not exhaustive, and that additional species within the scope of these defined terms may also be selected.

The term “halogen” represents chlorine, fluorine, bromine, or iodine. The term “halo” represents chloro, fluoro, bromo, or iodo.

The term “substituted” means that the specified group or moiety bears one or more substituents. The term “unsubstituted” means that the specified group bears no substituents. The term “optionally substituted” means that the specified group is unsubstituted or substituted by one or more substituents. Where the term “substituted” is used to describe a structural system, the substitution is meant to occur at any valency-allowed position on the system.

The term “amino acid substituent” as used in Formula (I) refers to the substituent on the carbon alpha to the carbonyl in any naturally occurring or synthetic amino acid. Exemplary amino acids include: arginine, histidine, lysine, aspartic acid, glutamic acid, serine, threonine, asparagine, glutamine, cysteine, selenocysteine, glycine, sarcosine, proline, alanine, isoleucine, leucine, norleucine, methionine, phenylalanine, tryptophan, tyrosine, valine, para-aminobenzoic acid, meta-aminobenzoic acid, and ortho-aminobenzoic acid. Thus, suitable “amino acid substituents” include, for example: H, pyrrolidine, or C₁₋₈alkyl optionally substituted with OH, amino, methylamino, —CO₂H, —CONH₂, SH, phenyl, hydroxyphenyl, indole, guanidine, SeH, or imidazole, and the like.

Any formula given herein is intended to represent compounds having structures depicted by the structural formula as well as certain variations or forms. In particular, compounds of any formula given herein may have asymmetric centers and therefore exist in different enantiomeric forms. All optical isomers and stereoisomers of the compounds of the general formula, and mixtures thereof, are considered within the scope of the formula. Thus, any formula given herein is intended to represent a racemate, one or more enantiomeric forms, one or more diastereomeric forms, one or more atropisomeric forms, and mixtures thereof. Furthermore, certain structures may exist as geometric isomers (i.e., cis and trans isomers), as tautomers, or as atropisomers. Additionally, any formula given herein is intended to refer also to any one of hydrates, solvates, and amorphous and polymorphic forms of such compounds, and mixtures thereof, even if such forms are not listed explicitly. In some embodiments, the solvent is water and the solvates are hydrates.

Any formula given herein is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the invention include isotopes of hydrogen, carbon, nitrogen, oxygen, phosphorous, fluorine, chlorine, and iodine, such as ²H, ³H, ¹¹C, ¹³C, ¹⁴C, ¹⁵N, ¹⁸O, ¹⁷O, ³¹P, ³²P, ³⁵S, ¹⁸F, ³⁶Cl, and ¹²⁵I, respectively. Such isotopically labeled compounds are useful in metabolic studies (preferably with ¹⁴C), reaction kinetic studies (with, for example ²H or ³H), detection or imaging techniques [such as positron emission tomography (PET) or single-photon emission computed tomography (SPECT)] including drug or substrate tissue distribution assays, or in radioactive treatment of patients. In particular, an ¹⁸F or ¹¹C labeled compound may be particularly preferred for PET or SPECT studies. Further, substitution with heavier isotopes such as deuterium (i.e., ²H) may afford certain therapeutic advantages resulting from greater metabolic stability, for example increased in vivo half-life or reduced dosage requirements. Isotopically labeled compounds of this invention and prodrugs thereof can generally be prepared by carrying out the procedures disclosed in the schemes or in the examples and preparations described below by substituting a readily available isotopically labeled reagent for a non-isotopically labeled reagent.

When referring to any formula given herein, the selection of a particular moiety from a list of possible species for a specified variable is not intended to define the same choice of the species for the variable appearing elsewhere. In other words, where a variable appears more than once, the choice of the species from a specified list is independent of the choice of the species for the same variable elsewhere in the formula, unless stated otherwise.

The nomenclature “Cᵢ₋ⱼ” with j>i, when applied herein to a class of substituents, is meant to refer to embodiments of this invention for which each and every one of the number of carbon members, from i to j including i and j, is independently realized. By way of example, the term C₁₋₃ refers independently to embodiments that have one carbon member (C₁), embodiments that have two carbon members (C₂), and embodiments that have three carbon members (C₃).

The term Cₙ₋ₘalkyl refers to an aliphatic chain, whether straight or branched, with a total number N of carbon members in the chain that satisfies n≦N≦m, with m>n.

Any disubstituent referred to herein is meant to encompass the various attachment possibilities when more than one of such possibilities are allowed. For example, reference to disubstituent -A-B-, where A B, refers herein to such disubstituent with A attached to a first substituted member and B attached to a second substituted member, and it also refers to such disubstituent with A attached to the second substituted member and B attached to the first substituted member.

Chemical names listed herein were generated using AutoNOM™ software. If there is a discrepancy between a chemical structure and the name listed for that structure, the structure prevails.

According to the foregoing interpretive considerations on assignments and nomenclature, it is understood that explicit reference herein to a set implies, where chemically meaningful and unless indicated otherwise, independent reference to embodiments of such set, and reference to each and every one of the possible embodiments of subsets of the set referred to explicitly.

In certain embodiments, R¹ and R² are each H or methyl. In other embodiments, R¹ is H and R² is methyl. In other embodiments, R¹ and R² are both methyl. In other embodiments, R¹ and R² is are each independently H or methyl.

In certain embodiments, R¹ and R² are taken together with the nitrogen to which they are attached to form an optionally substituted heterocyclic ring selected from the group consisting of pyrrole, imidazole, pyrrolidine, piperidine, morpholine, or thiomorpholine. In other embodiments, R¹ and R² are taken together with the nitrogen to which they are attached to form maleimido or 3,5-dioxo-3H-1,2,4-triazol-4(5H)-yl.

In certain embodiments, X is O. In other embodiments, X is absent.

In certain embodiments, R³ is H, pyrrolidine, or a C₁₋₈alkyl optionally substituted with OH, amino, methylamino, —CO₂H, —CONH₂, SH, phenyl, hydroxyphenyl, indole, guanidine, SeH, or imidazole. In other embodiments, R³ is isopropyl. In still other embodiments, R³ is (R)-isopropyl. In other embodiments, the group —(CH(R³))— is phenyl.

In certain embodiments, R⁴ is methyl. In certain embodiments, R⁵ is methyl. In other embodiments, R⁴ and R⁵ are both methyl.

In certain embodiments, R⁶ is H, methyl, ethyl, or isopropyl. In other embodiments, R⁶ is H.

In certain embodiments, R⁷ is H, methyl, ethyl, isopropyl, carboxy, carboxymethyl, methoxyethyloxy, dimethylaminoethylamino, boronate, dimethoxyboryl, or tetrazole. In other embodiments, R⁷ is H. In certain embodiments, R^{a}, R^{b}, and R^{c} are each independently H or methyl. In certain embodiments, m is 1.

In certain embodiments, n is 1, 2, or 3. In other embodiments, n is 1 or 2. In still other embodiments, n is 1.

In certain embodiments, Y is O. In other embodiments, Y is absent.

In certain embodiments, R⁸ is —NR^{d}R^{e} wherein R^{d} and R^{e} are each independently H, C₁₋₄alkyl, OH, SH, —NR^{f}R^{g}, —NHCOR^{g}, halo, —COR^{h}, optionally substituted phenyl, or an optionally substituted 3- to 8-membered heterocyclic ring. In other embodiments, R⁸ is —NR^{d}R^{e} wherein R^{d} and R^{e} are each independently H, C₁₋₄alkyl, OH, SH, —NR^{f}R^{g}, —NHCOR^{g}, halo, or —COR^{h}.

In other embodiments, R^{d} and R^{e} are each independently H, methyl, ethyl, isopropyl, OH, SH, amino, alkylamino, dialkylamino, —N(alkyl)(phenyl or heterocyclyl), —NHCOC₁₋₄alkyl, halo, carboxy, methoxycarbonyl, formyl, optionally substituted phenyl, or an optionally substituted 3- to 8-membered heterocyclic ring. In other embodiments, R^{d} and R^{e} are each independently H or methyl.

In certain embodiments, R^{f} and R^{g} are each independently H, methyl, ethyl, isopropyl, or phenyl. In further embodiments, R^{f} and R^{g} are each independently H or methyl.

In certain embodiments, R^{h} is H, —OH, —OCH₃, —NH₂, —NHNH₂, or —ONH₂.

In certain embodiments, R^{d} and R^{e} taken together with the nitrogen to which they are attached form a 5- to 8-membered heterocyclic ring, optionally substituted with one or two substituents selected from the group consisting of C₁₋₄alkyl, halo, OH, —OC₁₋₄alkyl, SH, —NR^{k}R^{l}, —NHC(O)R¹, cyano, —CONR^{k}R^{l}, —COR^{m}, phenyl, and a 3- to 8-membered heterocyclic ring.

In other embodiments, R^{d} and R^{e} taken together with the nitrogen to which they are attached form a heterocyclic ring which is optionally substituted with fluoro, chloro, OH, SH, amino, alkylamino, dialkylamino, C₁₋₄alkoxycarbonylamino, carboxy, methoxycarbonyl, —CONHNH₂, —COONH₂, or phenyl. In other embodiments, R^{d} and R^{e} taken together with the nitrogen to which they are attached form an optionally substituted heterocyclic ring selected from the group consisting of pyrrolidine, piperidine, morpholine, or thiomorpholine. In still other embodiments, R^{d} and R^{e} taken together with the nitrogen to which they are attached form piperazine, N-methylpiperazine, or morpholine. In other embodiments, R^{d} and R^{e} are taken together with the nitrogen to which they are attached to form maleimido or 3,5-dioxo-3H-1,2,4-triazol-4(5H)-yl. In other embodiments, R^{d} and R^{e} taken together with the nitrogen to which they are attached form piperazine, N-methylpiperazine, morpholine, maleimido, or 3,5-dioxo-3H-1,2,4-triazol-4(5H)-yl.

In other embodiments, R⁸ is a carbon-linked, nitrogen-containing heteroaryl ring, optionally substituted with one, two, or three substituents selected from the group consisting of halo, OH, SH, —NRⁿR^{o}, —NHCOR^{o}, —COR^{p}, phenyl, and a 3- to 8-membered heterocyclic ring. In other embodiments, R⁸ is an optionally substituted pyridine or imidazole ring. In other embodiments, R⁸ is 2-pyridyl, 3-pyridyl, 4-pyridyl, 1-imidazolyl, 2-imidazolyl, or 4-imidazolyl. In other embodiments, the R⁸ heteroaryl ring is substituted with fluoro, chloro, OH, SH, amino, alkylamino, dialkylamino, anilino, C₁₋₄alkoxycarbonylamino, carboxy, methoxycarbonyl, —CONHNH₂, —COONH₂, or phenyl.

In other embodiments, R⁸ is an optionally substituted phenyl ring. In other embodiments, R⁸ is phenyl, optionally substituted with fluoro, chloro, OH, SH, amino, alkylamino, dialkylamino, anilino, C₁₋₄alkoxycarbonylamino, carboxy, methoxycarbonyl, —CONHNH₂, —COONH₂, or phenyl. In other embodiments, R⁸ is phenyl, optionally substituted with amino.

In other embodiments, the compound of Formula (I) comprises an NH group which allows for connection to an antibody-linker moiety. In other embodiments, at least one of —NR¹R² and

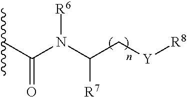

comprises an —NH moiety.

In some embodiments of Formula (I), the compound is not a compound wherein R¹ and R² are each H or methyl, R⁷ is H or —CO₂R^{a}, n is 1 or 2, Y is absent, and R⁸ is amino, alkylamino, dialkylamino, a 5- to 7-membered heterocyclic ring, or an optionally substituted phenyl or heteroaryl ring.

In certain embodiments, compounds of Formula (I) are selected from the group consisting of:
(S)—N-((3R,4S,5S)-3-methoxy-1-((S)-2-((1R,2R)-1-methoxy-2-methyl-3-oxo-3-((2-(pyridin-2-yl)ethyl)amino)propyl)pyrrolidin-1-yl)-5-methyl-1-oxoheptan-4-yl)-N,3-dimethyl-2-((S)-3-methyl-2-(methylamino)butanamido)butanamide;
4-amino-N—((S)-1-(((3R,4S,5S)-3-methoxy-1-((S)-2-((1R,2R)-1-methoxy-2-methyl-3-oxo-3-((2-(pyridin-2-yl)ethyl)amino)propyl)pyrrolidin-1-yl)-5-methyl-1-oxoheptan-4-yl)(methyl)amino)-3-methyl-1-oxobutan-2-yl)benzamide;
(S)—N-((3R,4S,5S)-3-methoxy-1-((S)-2-((1R,2R)-1-methoxy-2-methyl-3-oxo-3-((2-(pyridin-3-yl)ethyl)amino)propyl)pyrrolidin-1-yl)-5-methyl-1-oxoheptan-4-yl)-N,3-dimethyl-2-((S)-3-methyl-2-(methylamino)butanamido)butanamide;
4-amino-N—((S)-1-(((3R,4S,5S)-3-methoxy-1-((S)-2-((1R,2R)-1-methoxy-2-methyl-3-oxo-3-((2-(pyridin-3-yl)ethyl)amino)propyl)pyrrolidin-1-yl)-5-methyl-1-oxoheptan-4-yl)(methyl)amino)-3-methyl-1-oxobutan-2-yl)benzamide;
(S)—N-((3R,4S,5S)-3-methoxy-1-((S)-2-((1R,2R)-1-methoxy-2-methyl-3-oxo-3-((2-(pyridin-4-yl)ethyl)amino)propyl)pyrrolidin-1-yl)-5-methyl-1-oxoheptan-4-yl)-N,3-dimethyl-2-((S)-3-methyl-2-(methylamino)butanamido)butanamide;
4-amino-N—((S)-1-(((3R,4S,5S)-3-methoxy-1-((S)-2-((1R,2R)-1-methoxy-2-methyl-3-oxo-3-((2-(pyridin-4-yl)ethyl)amino)propyl)pyrrolidin-1-yl)-5-methyl-1-oxoheptan-4-yl)(methyl)amino)-3-methyl-1-oxobutan-2-yl)benzamide;
(S)—N-((3R,4S,5S)-1-((S)-2-((1R,2R)-3-((2-(1H-imidazol-1-yl)ethyl)amino)-1-methoxy-2-methyl-3-oxopropyl)pyrrolidin-1-yl)-3-methoxy-5-methyl-1-oxoheptan-4-yl)-N,3-dimethyl-2-((S)-3-methyl-2-(methylamino)butanamido)butanamide;
N—((S)-1-(((3R,4S,5S)-1-((S)-2-((1R,2R)-3-((2-(1H-imidazol-1-yl)ethyl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)-4-aminobenzamide;
(S)—N-((3R,4S,5S)-1-((S)-2-((1R,2R)-3-((2-(1H-imidazol-2-yl)ethyl)amino)-1-methoxy-2-methyl-3-oxopropyl)pyrrolidin-1-yl)-3-methoxy-5-methyl-1-oxoheptan-4-yl)-N,3-dimethyl-2-((S)-3-methyl-2-(methylamino)butanamido)butanamide;
N—((S)-1-(((3R,4S,5S)-1-((S)-2-((1R,2R)-3-((2-(1H-imidazol-2-yl)ethyl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)-4-aminobenzamide;
(S)—N-((3R,4S,5S)-1-((S)-2-((1R,2R)-3-((2-(1H-imidazol-4-yl)ethyl)amino)-1-methoxy-2-methyl-3-oxopropyl)pyrrolidin-1-yl)-3-methoxy-5-methyl-1-oxoheptan-4-yl)-N,3-dimethyl-2-((S)-3-methyl-2-(methylamino)butanamido)butanamide;
N—((S)-1-(((3R,4S,5S)-1-((S)-2-((1R,2R)-3-((2-(1H-imidazol-4-yl)ethyl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)-4-aminobenzamide;
(S)—N-((3R,4S,5S)-1-((S)-2-((R,2R)-3-((2-(dimethylamino)ethyl)amino)-1-methoxy-2-methyl-3-oxopropyl)pyrrolidin-1-yl)-3-methoxy-5-methyl-1-oxoheptan-4-yl)-N,3-dimethyl-2-((S)-3-methyl-2-(methylamino)butanamido)butanamide;
(S)-2-((S)-2-(dimethylamino)-3-methylbutanamido)-N-((3R,4S,5S)-3-methoxy-1-((S)-2-((R,2R)-1-methoxy-2-methyl-3-((2-(methylamino)ethyl)amino)-3-oxopropyl)pyrrolidin-1-yl)-5-methyl-1-oxoheptan-4-yl)-N,3-dimethylbutanamide;
(S)—N-((3R,4S,5S)-1-((S)-2-((R,2R)-3-((2-aminoethyl)amino)-1-methoxy-2-methyl-3-oxopropyl)pyrrolidin-1-yl)-3-methoxy-5-methyl-1-oxoheptan-4-yl)-2-((S)-2-(dimethylamino)-3-methylbutanamido)-N,3-dimethylbutanamide;
(S)—N-((3R,4S,5S)-3-methoxy-1-((S)-2-((1R,2R)-1-methoxy-2-methyl-3-((2-morpholinoethyl)amino)-3-oxopropyl)pyrrolidin-1-yl)-5-methyl-1-oxoheptan-4-yl)-N,3-dimethyl-2-((S)-3-methyl-2-(methylamino)butanamido)butanamide;
(S)-2-((S)-2-(dimethylamino)-3-methylbutanamido)-N-((3R,4S,5S)-3-methoxy-1-((S)-2-((R,2R)-1-methoxy-2-methyl-3-oxo-3-((2-(piperazin-1-yl)ethyl)amino)propyl)pyrrolidin-1-yl)-5-methyl-1-oxoheptan-4-yl)-N,3-dimethylbutanamide;
(S)—N-((3R,4S,5S)-3-methoxy-1-((S)-2-((1R,2R)-1-methoxy-2-methyl-3-oxo-3-((2-(piperazin-1-yl)ethyl)amino)propyl)pyrrolidin-1-yl)-5-methyl-1-oxoheptan-4-yl)-N,3-dimethyl-2-((S)-3-methyl-2-(methylamino)butanamido)butanamide;
(S)—N-((3R,4S,5S)-3-methoxy-1-((S)-2-((1R,2R)-1-methoxy-2-methyl-3-((2-(4-methylpiperazin-1-yl)ethyl)amino)-3-oxopropyl)pyrrolidin-1-yl)-5-methyl-1-oxoheptan-4-yl)-N,3-dimethyl-2-((S)-3-methyl-2-(methylamino)butanamido)butanamide;
4-amino-N—((S)-1-(((3R,4S,5S)-3-methoxy-1-((S)-2-((1R,2R)-1-methoxy-2-methyl-3-((2-morpholinoethyl)amino)-3-oxopropyl)pyrrolidin-1-yl)-5-methyl-1-oxoheptan-4-yl)(methyl)amino)-3-methyl-1-oxobutan-2-yl)benzamide;
4-amino-N—((S)-1-(((3R,4S,5S)-3-methoxy-1-((S)-2-((1R,2R)-1-methoxy-2-methyl-3-oxo-3-((2-(piperazin-1-yl)ethyl)amino)propyl)pyrrolidin-1-yl)-5-methyl-1-oxoheptan-4-yl)(methyl)amino)-3-methyl-1-oxobutan-2-yl)benzamide; and
(S)—N-((3R,4S,5S)-1-((S)-2-((R,2R)-3-((4-aminophenethyl)amino)-1-methoxy-2-methyl-3-oxopropyl)pyrrolidin-1-yl)-3-methoxy-5-methyl-1-oxoheptan-4-yl)-2-((S)-2-(dimethylamino)-3-methylbutanamido)-N,3-dimethylbutanamide;
and pharmaceutically acceptable salts thereof.

In other embodiments, the compound of Formula (I) is (S)—N-((3R,4S,5S)-3-methoxy-1-((S)-2-((1R,2R)-1-methoxy-2-methyl-3-oxo-3-((2-(pyridin-2-yl)ethyl)amino)propyl)pyrrolidin-1-yl)-5-methyl-1-oxoheptan-4-yl)-N,3-dimethyl-2-((S)-3-methyl-2-(methylamino)butanamido)butanamide or (S)—N-((3R,4S,5S)-1-((S)-2-((1R,2R)-3-((4-aminophenethyl)amino)-1-methoxy-2-methyl-3-oxopropyl)pyrrolidin-1-yl)-3-methoxy-5-methyl-1-oxoheptan-4-yl)-2-((S)-2-(dimethylamino)-3-methylbutanamido)-N,3-dimethylbutanamide, or a pharmaceutically acceptable salt thereof.

The present invention is also directed toward embodiments of Formula (II) wherein each of the variables is independently defined as indicated for Formula (I).

The invention includes pharmaceutically acceptable salts of the compounds of Formula (I), preferably of those described above and the specific compounds exemplified herein, pharmaceutical compositions comprising such salts, and methods of using such salts.

A “pharmaceutically acceptable salt” is intended to mean a salt of a free acid or base of a compound represented herein that is non-toxic, biologically tolerable, or otherwise biologically suitable for administration to the subject. See, generally, S. M. Berge, et al. “Pharmaceutical Salts,” J. Pharm. Sci. 1977, 66, 1-19. Preferred pharmaceutically acceptable salts are those that are pharmacologically effective and suitable for contact with the tissues of subjects without undue toxicity, irritation, or allergic response. A compound described herein may possess a sufficiently acidic group, a sufficiently basic group, or both types of functional groups, and accordingly react with a number of inorganic or organic bases, and inorganic and organic acids, to form a pharmaceutically acceptable salt. Examples of pharmaceutically acceptable salts include sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, methylsulfonates, propylsulfonates, besylates, xylenesulfonates, naphthalene-1-sulfonates, naphthalene-2-sulfonates, phenylacetates, phenylpropionates, phenylbutyrates, citrates, lactates, α-hydroxybutyrates, glycolates, tartrates, and mandelates.

The term “antibody” is used in the broadest sense unless clearly indicated otherwise. Therefore, an “antibody” can be naturally occurring or man-made such as monoclonal antibodies produced by conventional hybridoma technology. Suitable antibodies comprise monoclonal and polyclonal antibodies as well as fragments containing the antigen-binding domain and/or one or more complementarity determining regions of these antibodies. As used herein, the term “antibody” refers to any form of antibody, or fragment thereof, that specifically binds to a target antigen and/or exhibits the desired biological activity and specifically covers monoclonal antibodies (including full length monoclonal antibodies), polyclonal antibodies, multispecific antibodies (e.g., bispecific antibodies), and antibody fragments, so long as they specifically bind to the target antigen and/or exhibit the desired biological activity.

Any specific antibody can be used in the methods and compositions provided herein. Thus, in one embodiment the term “antibody” encompasses a molecule comprising at least one variable region from a light chain immunoglobulin molecule and at least one variable region from a heavy chain molecule that in combination form a specific binding site for the target antigen. In one embodiment, the antibody is an IgG antibody. For example, the antibody is an IgG1, IgG2, IgG3, or IgG4 antibody.

The antibodies useful in the present methods and compositions can be generated in cell culture, in phage, or in various animals, including but not limited to cows, rabbits, goats, mice, rats, hamsters, guinea pigs, sheep, dogs, cats, monkeys, chimpanzees, and apes. Therefore, in one embodiment, an antibody useful in the present invention is a mammalian antibody. Phage techniques can be used to isolate an initial antibody or to generate variants with altered specificity or avidity characteristics. Such techniques are routine and well known in the art. In one embodiment, the antibody is produced by recombinant means known in the art. For example, a recombinant antibody can be produced by transfecting a host cell with a vector comprising a DNA sequence encoding the antibody. One or more vectors can be used to transfect the DNA sequence expressing at least one VL and one VH region in the host cell. Exemplary descriptions of recombinant means of antibody generation and production include Delves, Antibody Production: Essential Techniques (Wiley, 1997); Shephard et al. Monoclonal Antibodies (Oxford University Press, 2000); Goding, Monoclonal Antibodies: Principles and Practice (Academic Press, 1993); and Current Protocols in Immunology (John Wiley & Sons, most recent edition). An antibody useful in the present invention can be modified by recombinant means to increase efficacy of the antibody in mediating the desired function. Thus, it is within the scope of the invention that antibodies can be modified by substitutions using recombinant means. Typically, the substitutions will be conservative substitutions. For example, at least one amino acid in the constant region of the antibody can be replaced with a different residue. See, e.g., U.S. Pat. No. 5,624,821, U.S. Pat. No. 6,194,551, Application No. WO 9958572; and Angal et al. (1993) Mol. Immunol. 30:105-08. Suitable amino acid modifications include deletions, additions, and substitutions of amino acids. In some cases, such changes are made to reduce undesired activities, e.g., complement-dependent cytotoxicity or to improve CMC properties. Frequently, the antibodies are labeled by joining, either covalently or non-covalently, a substance which provides for a detectable signal. A wide variety of labels and conjugation techniques are known and are reported extensively in both the scientific and patent literature. These antibodies can be screened for binding to normal or defective targets. See e.g., Antibody Engineering: A Practical Approach (Oxford University Press, 1996).

In one embodiment, an antibody useful in the present invention is a “human antibody.” As used herein, the term “human antibody” refers to an antibody in which essentially the entire sequences of the light chain and heavy chain sequences, including the complementary determining regions (CDRs), are from human genes. In one embodiment, human monoclonal antibodies are prepared by the trioma technique, the human B-cell technique (see, e.g., Kozbor, et al. (1983) Immunol. Today 4:72, EBV transformation technique (see, e.g., Cole et al. Monoclonal Antibodies and Cancer Therapy (1985) 77-96), or using phage display (see, e.g., Marks et al. (1991) J. Mol. Biol. 222:581). In a specific embodiment, the human antibody is generated in a transgenic mouse. Techniques for making such partially to fully human antibodies are known in the art and any such techniques can be used. According to one particularly preferred embodiment, fully human antibody sequences are made in a transgenic mouse engineered to express human heavy and light chain antibody genes. An exemplary description of preparing transgenic mice that produce human antibodies found in Application No. WO 02/43478 and U.S. Pat. No. 6,657,103 (Abgenix) and its progeny. B cells from transgenic mice that produce the desired antibody can then be fused to make hybridoma cell lines for continuous production of the antibody. See, e.g., U.S. Pat. Nos. 5,569,825; 5,625,126; 5,633,425; 5,661,016; and 5,545,806; and Jakobovits (1998) Adv. Drug Del. Rev. 31:33-42; Green et al. (1998) J. Exp. Med. 188:483-95.

As used herein, the term “humanized antibody” refers to forms of antibodies that contain sequences from non-human (e.g., murine) antibodies as well as human antibodies. Such antibodies are chimeric antibodies which contain minimal sequence derived from non-human immunoglobulin. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. See e.g., Cabilly, U.S. Pat. No. 4,816,567; Queen et al. (1989) Proc. Natl. Acad. Sci. USA 86:10029-10033; and Antibody Engineering: A Practical Approach (Oxford University Press 1996).

The term “monoclonal antibody,” as used herein, refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic epitope. In contrast, conventional (polyclonal) antibody preparations typically include a multitude of antibodies directed against (or specific for) different epitopes. In one embodiment, the polyclonal antibody contains a plurality of monoclonal antibodies with different epitope specificities, affinities, or avidities within a single antigen that contains multiple antigenic epitopes. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al. (1975) Nature 256: 495, or may be made by recombinant DNA methods (see, e.g., U.S. Pat. No. 4,816,567). The “monoclonal antibodies” may also be isolated from phage antibody libraries using the techniques described in Clackson et al. (1991) Nature 352: 624-628 and Marks et al. (1991) J. Mol. Biol. 222: 581-597, for example. These monoclonal antibodies will usually bind with at least a K_{d} of about 1 μM, more usually at least about 300 nM, typically at least about 30 nM, preferably at least about 10 nM, more preferably at least about 3 nM or better, usually determined by ELISA, FACS or BIACORE.

In a preferred embodiment, the antibody is a fully human antibody.

For compositions comprising a plurality of antibodies, the drug loading is represented by q, the average number of drug molecules per antibody. Drug loading may range from 1 to 20 drugs (D) per antibody. The average number of drugs per antibody in preparation of conjugation reactions may be characterized by conventional means such as mass spectroscopy, ELISA assay, and HPLC. The quantitative distribution of Antibody-Drug-Conjugates in terms of p may also be determined. In some instances, separation, purification, and characterization of homogeneous antibody-drug conjugates where q is a certain value from antibody-drug conjugates with other drug loadings may be achieved by means such as reverse phase HPLC or electrophoresis. In exemplary embodiments, q is from 2 to 8.

As shown in Formula II, antibody-drug conjugate compounds comprise a linker unit between the drug unit and the antibody unit. As used herein, the term “linker” refers to a bifunctional compound that can be used to link a compound of Formula (I) to an antibody to form an antibody-drug conjugate of Formula (II). A variety of linkers can be used with the present compositions. For example, exemplary linkers, including their structure and synthesis, are described in WO 2004/010957, U.S. Pat. Publ. No. 2006/0074008, U.S. Pat. Publ. No. 2005/0238649, and U.S. Pat. Publ. No. 2006/0024317, U.S. Pat. Publ. Nos. 2003/0083263, 2005/0238649 and 2005/0009751, each of which is incorporated herein by reference in its entirety and for all purposes.

In some embodiments, the linker is cleavable under intracellular conditions, such that cleavage of the linker releases the drug unit from the antibody in the intracellular environment. In yet other embodiments, the linker unit is not cleavable and the drug is released, for example, by antibody degradation.

In some embodiments, the linker is cleavable by a cleaving agent that is present in the intracellular environment (e.g., within a lysosome or endosome or caveolea). The linker can be, e.g., a peptidyl linker that is cleaved by an intracellular peptidase or protease enzyme, including, but not limited to, a lysosomal or endosomal protease. In some embodiments, the peptidyl linker is at least two amino acids long or at least three amino acids long. Cleaving agents can include cathepsins B and D and plasmin, all of which are known to hydrolyze dipeptide drug derivatives resulting in the release of active drug inside target cells (see, e.g., Dubowchik and Walker (1999) Pharm. Therapeutics 83:67-123). Most typical are peptidyl linkers that are cleavable by enzymes that are present in tumor cells expressing the target antigen. For example, a peptidyl linker that is cleavable by the thiol-dependent protease cathepsin-B, which is highly expressed in cancerous tissue, can be used (e.g., a Phe-Leu containing linker). Other examples of such linkers are described, e.g., in U.S. Pat. No. 6,214,345, incorporated herein by reference in its entirety and for all purposes. In a specific embodiment, the peptidyl linker cleavable by an intracellular protease is a Val-Cit linker or a Phe-Lys linker (see, e.g., U.S. Pat. No. 6,214,345, which describes the synthesis of doxorubicin with the val-cit linker). One advantage of using intracellular proteolytic release of the therapeutic agent is that the agent is typically attenuated when conjugated and the serum stabilities of the conjugates are typically high.

In other embodiments, the cleavable linker is pH-sensitive, i.e., sensitive to hydrolysis at certain pH values. Typically, the pH-sensitive linker hydrolyzable under acidic conditions. For example, an acid-labile linker that is hydrolyzable in the lysosome (e.g., a hydrazone, semicarbazone, thiosemicarbazone, cis-aconitic amide, orthoester, acetal, ketal, or the like) can be used. (See, e.g., U.S. Pat. Nos. 5,122,368; 5,824,805; 5,622,929; Dubowchik and Walker (1999), supra; Neville et al. (1989) Biol. Chem. 264:14653-14661.) Such linkers are relatively stable under neutral pH conditions, such as those in the blood, but are unstable at below pH 5.5 or 5.0, the approximate pH of the lysosome. In certain embodiments, the hydrolyzable linker is a thioether linker (such as, e.g., a thioether attached to the therapeutic agent via an acylhydrazone bond (see, e.g., U.S. Pat. No. 5,622,929).

In yet other specific embodiments, the linker is a malonate linker (Johnson et al. (1995) Anticancer Res. 15:1387-93), a maleimidobenzoyl linker (Lau et al. (1995) Bioorg. Med. Chem. 3(10):1299-1304), or a 3′-N-amide analog (Lau et al. (1995) Bioorg. Med. Chem. 3(10):1305-12).

In yet other embodiments, the linker unit is not cleavable and the drug is released by antibody degradation. (See U.S. Pat. Publ. No. 2005/0238649 incorporated by reference herein in its entirety and for all purposes).

Typically, the linker is not substantially sensitive to the extracellular environment. As used herein, “not substantially sensitive to the extracellular environment,” in the context of a linker, means that no more than about 20%, typically no more than about 15%, more typically no more than about 10%, and even more typically no more than about 5%, no more than about 3%, or no more than about 1% of the linkers, in a sample of antibody-drug conjugate compound, are cleaved when the antibody-drug conjugate compound presents in an extracellular environment (e.g., in plasma). Whether a linker is not substantially sensitive to the extracellular environment can be determined, for example, by incubating the antibody-drug conjugate with plasma for a predetermined time period (e.g., 2, 4, 8, 16, or 24 hours) and then quantitating the amount of free drug present in the plasma.

In other embodiments, conjugates of the antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al (1987) Science, 238:1098. Carbon-14-labeled 1-isothiocyanatobenzyl-3-methyldiethylene triaminepentaacetic acid (MX-DTPA) is an exemplary chelating agent for conjugation of radionucleotide to the antibody (WO94/11026).

A number of different reactions are available for covalent attachment of drugs and/or linkers to toxins. This is often accomplished by reaction of the amino acid residues of the binding agent, e.g., antibody molecule, including the amine groups of lysine, the free carboxylic acid groups of glutamic and aspartic acid, the sulfhydryl groups of cysteine and the various moieties of the aromatic amino acids. One of the most commonly used non-specific methods of covalent attachment is the carbodiimide reaction to link a carboxy (or amino) group of a compound to amino (or carboxy) groups of the antibody. Additionally, bifunctional agents such as dialdehydes or imidoesters have been used to link the amino group of a compound to amino groups of an antibody molecule. Also available for attachment of drugs to binding agents is the Schiff base reaction. This method involves the periodate oxidation of a drug that contains glycol or hydroxy groups, thus forming an aldehyde which is then reacted with the binding agent. Attachment occurs via formation of a Schiff base with amino groups of the binding agent. Isothiocyanates can also be used as coupling agents for covalently attaching drugs to binding agents. Other techniques are known to the skilled artisan and within the scope of the present invention.

In some embodiments, the toxins described herein are linked to antibodies as described for N-terminus linked monomethylauristatin drug moieties DE and DF, disclosed in Senter et al, Proceedings of the American Association for Cancer Research, Volume 45, Abstract Number 623, presented Mar. 28, 2004 and described in U.S. Pat. No. Publication No. 2005/0238649, the disclosure of which is expressly incorporated by reference in its entirety.

In other, non-mutually exclusive embodiments, the linker promotes cellular internalization. In certain embodiments, the linker promotes cellular internalization when conjugated to the therapeutic agent.

For treatment purposes, pharmaceutical compositions comprising compounds or conjugates of the invention may further comprise one or more pharmaceutically-acceptable excipients. A pharmaceutically-acceptable excipient is a substance that is non-toxic and otherwise biologically suitable for administration to a subject. Such excipients facilitate formulation and administration of a compound or conjugate of the invention and are compatible with the active ingredient. Examples of pharmaceutically-acceptable excipients include stabilizers, lubricants, surfactants, diluents, anti-oxidants, binders, coloring agents, emulsifiers, or taste-modifying agents. In preferred embodiments, pharmaceutical compositions are sterile compositions.

The pharmaceutical compositions described herein may be formulated as solutions, emulsions, suspensions, or dispersions in suitable pharmaceutical solvents or carriers, or as pills, tablets, lozenges, suppositories, powders for reconstitution, or capsules along with solid carriers according to conventional methods known in the art for preparation of various dosage forms. For topical applications, the compounds and conjugates described herein are preferably formulated as creams or ointments or a similar vehicle suitable for topical administration. The pharmaceutical compositions and compounds and conjugates described herein may be administered in the inventive methods by a suitable route of delivery, e.g., oral, nasal, parenteral, rectal, topical, ocular, or by inhalation.

The term “treat” or “treating” as used herein is intended to refer to administration of a compound of the present invention to a subject for the purpose of creating a therapeutic benefit. Treating includes reversing, ameliorating, alleviating, inhibiting the progress of, or lessening the severity of, a disease, disorder, or condition, or one or more symptoms of cancer. The term “subject” refers to a mammalian patient in need of such treatment, such as a human.

In treatment methods according to the invention, “an effective amount” means an amount or dose sufficient to generally bring about the desired therapeutic benefit in subjects needing such treatment. Effective amounts or doses of the compounds or conjugates described herein may be ascertained by routine methods, such as modeling, dose escalation or clinical trials, taking into account routine factors, e.g., the mode or route of administration or drug delivery, the pharmacokinetics of the agent, the severity and course of the infection, the subject's health status, condition, and weight, and the judgment of the treating physician. An exemplary dose is in the range of about 1 ug to 2 mg of active compound per kilogram of subject's body weight per day, preferably about 0.05 to 100 mg/kg/day, or about 1 to 35 mg/kg/day, or about 0.1 to 10 mg/kg/day. The total dosage may be given in single or divided dosage units (e.g., BID, TID, QID). In the context of drug-antibody conjugates, a suitable dose is in the range of 1 to 10 mg per kilogram of the subject's body weight per dose, or from 3 to 8 mg per kilogram, or about 5 mg per kilogram, with administration of from 1 to 7 doses per day.

The compounds and conjugates described herein may be used pharmaceutical compositions or methods in combination with additional active ingredients in the treatment of cancer. The additional active ingredients may be administered separately from a described compound or conjugate of the invention or may be included with a compound or conjugate of the invention in a pharmaceutical composition according to the invention. For example, additional active ingredients are those that are known or discovered to be effective in treating cancer, including those active against another target associated with cancer, such as but not limited to, Velcade, Rituximab, Methotrexate, Herceptin, Vincristine, Prednisone, Irinotecan, or the like, or a combination thereof. Such a combination may serve to increase efficacy, decrease one or more side effects, or decrease the required dose of a disclosed compound or conjugate.

In certain embodiments, the pharmaceutical composition of the invention comprises an effective amount of at least one compound selected from the group consisting of:
(S)—N-((3R,4S,5S)-3-methoxy-1-((S)-2-((1R,2R)-1-methoxy-2-methyl-3-oxo-3-((2-(pyridin-2-yl)ethyl)amino)propyl)pyrrolidin-1-yl)-5-methyl-1-oxoheptan-4-yl)-N,3-dimethyl-2-((S)-3-methyl-2-(methylamino)butanamido)butanamide;
4-amino-N—((S)-1-(((3R,4S,5S)-3-methoxy-1-((S)-2-((1R,2R)-1-methoxy-2-methyl-3-oxo-3-((2-(pyridin-2-yl)ethyl)amino)propyl)pyrrolidin-1-yl)-5-methyl-1-oxoheptan-4-yl)(methyl)amino)-3-methyl-1-oxobutan-2-yl)benzamide;
(S)—N-((3R,4S,5S)-3-methoxy-1-((S)-2-((1R,2R)-1-methoxy-2-methyl-3-oxo-3-((2-(pyridin-3-yl)ethyl)amino)propyl)pyrrolidin-1-yl)-5-methyl-1-oxoheptan-4-yl)-N,3-dimethyl-2-((S)-3-methyl-2-(methylamino)butanamido)butanamide;
4-amino-N—((S)-1-(((3R,4S,5S)-3-methoxy-1-((S)-2-((1R,2R)-1-methoxy-2-methyl-3-oxo-3-((2-(pyridin-3-yl)ethyl)amino)propyl)pyrrolidin-1-yl)-5-methyl-1-oxoheptan-4-yl)(methyl)amino)-3-methyl-1-oxobutan-2-yl)benzamide;
(S)—N-((3R,4S,5S)-3-methoxy-1-((S)-2-((1R,2R)-1-methoxy-2-methyl-3-oxo-3-((2-(pyridin-4-yl)ethyl)amino)propyl)pyrrolidin-1-yl)-5-methyl-1-oxoheptan-4-yl)-N,3-dimethyl-2-((S)-3-methyl-2-(methylamino)butanamido)butanamide;
4-amino-N—((S)-1-(((3R,4S,5S)-3-methoxy-1-((S)-2-((1R,2R)-1-methoxy-2-methyl-3-oxo-3-((2-(pyridin-4-yl)ethyl)amino)propyl)pyrrolidin-1-yl)-5-methyl-1-oxoheptan-4-yl)(methyl)amino)-3-methyl-1-oxobutan-2-yl)benzamide;
(S)—N-((3R,4S,5S)-1-((S)-2-((1R,2R)-3-((2-(1H-imidazol-1-yl)ethyl)amino)-1-methoxy-2-methyl-3-oxopropyl)pyrrolidin-1-yl)-3-methoxy-5-methyl-1-oxoheptan-4-yl)-N,3-dimethyl-2-((S)-3-methyl-2-(methylamino)butanamido)butanamide;
N—((S)-1-(((3R,4S,5S)-1-((S)-2-((1R,2R)-3-((2-(1H-imidazol-1-yl)ethyl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)-4-aminobenzamide;
(S)—N-((3R,4S,5S)-1-((S)-2-((1R,2R)-3-((2-(1H-imidazol-2-yl)ethyl)amino)-1-methoxy-2-methyl-3-oxopropyl)pyrrolidin-1-yl)-3-methoxy-5-methyl-1-oxoheptan-4-yl)-N,3-dimethyl-2-((S)-3-methyl-2-(methylamino)butanamido)butanamide;
N—((S)-1-(((3R,4S,5S)-1-((S)-2-((1R,2R)-3-((2-(1H-imidazol-2-yl)ethyl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)-4-aminobenzamide;
(S)—N-((3R,4S,5S)-1-((S)-2-((1R,2R)-3-((2-(1H-imidazol-4-yl)ethyl)amino)-1-methoxy-2-methyl-3-oxopropyl)pyrrolidin-1-yl)-3-methoxy-5-methyl-1-oxoheptan-4-yl)-N,3-dimethyl-2-((S)-3-methyl-2-(methylamino)butanamido)butanamide;
N—((S)-1-(((3R,4S,5S)-1-((S)-2-((1R,2R)-3-((2-(1H-imidazol-4-yl)ethyl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)-4-aminobenzamide;
(S)—N-((3R,4S,5S)-1-((S)-2-((R,2R)-3-((2-(dimethylamino)ethyl)amino)-1-methoxy-2-methyl-3-oxopropyl)pyrrolidin-1-yl)-3-methoxy-5-methyl-1-oxoheptan-4-yl)-N,3-dimethyl-2-((S)-3-methyl-2-(methylamino)butanamido)butanamide;
(S)-2-((S)-2-(dimethylamino)-3-methylbutanamido)-N-((3R,4S,5S)-3-methoxy-1-((S)-2-((R,2R)-1-methoxy-2-methyl-3-((2-(methylamino)ethyl)amino)-3-oxopropyl)pyrrolidin-1-yl)-5-methyl-1-oxoheptan-4-yl)-N,3-dimethylbutanamide;
(S)—N-((3R,4S,5S)-1-((S)-2-((R,2R)-3-((2-aminoethyl)amino)-1-methoxy-2-methyl-3-oxopropyl)pyrrolidin-1-yl)-3-methoxy-5-methyl-1-oxoheptan-4-yl)-2-((S)-2-(dimethylamino)-3-methylbutanamido)-N,3-dimethylbutanamide;
(S)—N-((3R,4S,5S)-3-methoxy-1-((S)-2-((1R,2R)-1-methoxy-2-methyl-3-((2-morpholinoethyl)amino)-3-oxopropyl)pyrrolidin-1-yl)-5-methyl-1-oxoheptan-4-yl)-N,3-dimethyl-2-((S)-3-methyl-2-(methylamino)butanamido)butanamide;
(S)-2-((S)-2-(dimethylamino)-3-methylbutanamido)-N-((3R,4S,5S)-3-methoxy-1-((S)-2-((R,2R)-1-methoxy-2-methyl-3-oxo-3-((2-(piperazin-1-yl)ethyl)amino)propyl)pyrrolidin-1-yl)-5-methyl-1-oxoheptan-4-yl)-N,3-dimethylbutanamide;
(S)—N-((3R,4S,5S)-3-methoxy-1-((S)-2-((1R,2R)-1-methoxy-2-methyl-3-oxo-3-((2-(piperazin-1-yl)ethyl)amino)propyl)pyrrolidin-1-yl)-5-methyl-1-oxoheptan-4-yl)-N,3-dimethyl-2-((S)-3-methyl-2-(methylamino)butanamido)butanamide;
(S)—N-((3R,4S,5S)-3-methoxy-1-((S)-2-((1R,2R)-1-methoxy-2-methyl-3-((2-(4-methylpiperazin-1-yl)ethyl)amino)-3-oxopropyl)pyrrolidin-1-yl)-5-methyl-1-oxoheptan-4-yl)-N,3-dimethyl-2-((S)-3-methyl-2-(methylamino)butanamido)butanamide;
4-amino-N—((S)-1-(((3R,4S,5S)-3-methoxy-1-((S)-2-((1R,2R)-1-methoxy-2-methyl-3-((2-morpholinoethyl)amino)-3-oxopropyl)pyrrolidin-1-yl)-5-methyl-1-oxoheptan-4-yl)(methyl)amino)-3-methyl-1-oxobutan-2-yl)benzamide;
4-amino-N—((S)-1-(((3R,4S,5S)-3-methoxy-1-((S)-2-((1R,2R)-1-methoxy-2-methyl-3-oxo-3-((2-(piperazin-1-yl)ethyl)amino)propyl)pyrrolidin-1-yl)-5-methyl-1-oxoheptan-4-yl)(methyl)amino)-3-methyl-1-oxobutan-2-yl)benzamide; and
(S)—N-((3R,4S,5S)-1-((S)-2-((R,2R)-3-((4-aminophenethyl)amino)-1-methoxy-2-methyl-3-oxopropyl)pyrrolidin-1-yl)-3-methoxy-5-methyl-1-oxoheptan-4-yl)-2-((S)-2-(dimethylamino)-3-methylbutanamido)-N,3-dimethylbutanamide;
and pharmaceutically acceptable salts thereof.

In certain embodiments, the method of treating according to the invention comprises administering at least one compound selected from the group consisting of:
(S)—N-((3R,4S,5S)-3-methoxy-1-((S)-2-((1R,2R)-1-methoxy-2-methyl-3-oxo-3-((2-(pyridin-2-yl)ethyl)amino)propyl)pyrrolidin-1-yl)-5-methyl-1-oxoheptan-4-yl)-N,3-dimethyl-2-((S)-3-methyl-2-(methylamino)butanamido)butanamide;
4-amino-N—((S)-1-(((3R,4S,5S)-3-methoxy-1-((S)-2-((1R,2R)-1-methoxy-2-methyl-3-oxo-3-((2-(pyridin-2-yl)ethyl)amino)propyl)pyrrolidin-1-yl)-5-methyl-1-oxoheptan-4-yl)(methyl)amino)-3-methyl-1-oxobutan-2-yl)benzamide;
(S)—N-((3R,4S,5S)-3-methoxy-1-((S)-2-((1R,2R)-1-methoxy-2-methyl-3-oxo-3-((2-(pyridin-3-yl)ethyl)amino)propyl)pyrrolidin-1-yl)-5-methyl-1-oxoheptan-4-yl)-N,3-dimethyl-2-((S)-3-methyl-2-(methylamino)butanamido)butanamide;
4-amino-N—((S)-1-(((3R,4S,5S)-3-methoxy-1-((S)-2-((1R,2R)-1-methoxy-2-methyl-3-oxo-3-((2-(pyridin-3-yl)ethyl)amino)propyl)pyrrolidin-1-yl)-5-methyl-1-oxoheptan-4-yl)(methyl)amino)-3-methyl-1-oxobutan-2-yl)benzamide;
(S)—N-((3R,4S,5S)-3-methoxy-1-((S)-2-((1R,2R)-1-methoxy-2-methyl-3-oxo-3-((2-(pyridin-4-yl)ethyl)amino)propyl)pyrrolidin-1-yl)-5-methyl-1-oxoheptan-4-yl)-N,3-dimethyl-2-((S)-3-methyl-2-(methylamino)butanamido)butanamide;
4-amino-N—((S)-1-(((3R,4S,5S)-3-methoxy-1-((S)-2-((1R,2R)-1-methoxy-2-methyl-3-oxo-3-((2-(pyridin-4-yl)ethyl)amino)propyl)pyrrolidin-1-yl)-5-methyl-1-oxoheptan-4-yl)(methyl)amino)-3-methyl-1-oxobutan-2-yl)benzamide;
(S)—N-((3R,4S,5S)-1-((S)-2-((1R,2R)-3-((2-(1H-imidazol-1-yl)ethyl)amino)-1-methoxy-2-methyl-3-oxopropyl)pyrrolidin-1-yl)-3-methoxy-5-methyl-1-oxoheptan-4-yl)-N,3-dimethyl-2-((S)-3-methyl-2-(methylamino)butanamido)butanamide;
N—((S)-1-(((3R,4S,5S)-1-((S)-2-((1R,2R)-3-((2-(1H-imidazol-1-yl)ethyl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)-4-aminobenzamide;
(S)—N-((3R,4S,5S)-1-((S)-2-((1R,2R)-3-((2-(1H-imidazol-2-yl)ethyl)amino)-1-methoxy-2-methyl-3-oxopropyl)pyrrolidin-1-yl)-3-methoxy-5-methyl-1-oxoheptan-4-yl)-N,3-dimethyl-2-((S)-3-methyl-2-(methylamino)butanamido)butanamide;
N—((S)-1-(((3R,4S,5S)-1-((S)-2-((1R,2R)-3-((2-(1H-imidazol-2-yl)ethyl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)-4-aminobenzamide;
(S)—N-((3R,4S,5S)-1-((S)-2-((1R,2R)-3-((2-(1H-imidazol-4-yl)ethyl)amino)-1-methoxy-2-methyl-3-oxopropyl)pyrrolidin-1-yl)-3-methoxy-5-methyl-1-oxoheptan-4-yl)-N,3-dimethyl-2-((S)-3-methyl-2-(methylamino)butanamido)butanamide;
N—((S)-1-(((3R,4S,5S)-1-((S)-2-((1R,2R)-3-((2-(1H-imidazol-4-yl)ethyl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)-4-aminobenzamide;
(S)—N-((3R,4S,5S)-1-((S)-2-((1R,2R)-3-((2-(dimethylamino)ethyl)amino)-1-methoxy-2-methyl-3-oxopropyl)pyrrolidin-1-yl)-3-methoxy-5-methyl-1-oxoheptan-4-yl)-N,3-dimethyl-2-((S)-3-methyl-2-(methylamino)butanamido)butanamide;
(S)-2-((S)-2-(dimethylamino)-3-methylbutanamido)-N-((3R,4S,5S)-3-methoxy-1-((S)-2-((R,2R)-1-methoxy-2-methyl-3-((2-(methylamino)ethyl)amino)-3-oxopropyl)pyrrolidin-1-yl)-5-methyl-1-oxoheptan-4-yl)-N,3-dimethylbutanamide;
(S)—N-((3R,4S,5S)-1-((S)-2-((R,2R)-3-((2-aminoethyl)amino)-1-methoxy-2-methyl-3-oxopropyl)pyrrolidin-1-yl)-3-methoxy-5-methyl-1-oxoheptan-4-yl)-2-((S)-2-(dimethylamino)-3-methylbutanamido)-N,3-dimethylbutanamide;
(S)—N-((3R,4S,5S)-3-methoxy-1-((S)-2-((1R,2R)-1-methoxy-2-methyl-3-((2-morpholinoethyl)amino)-3-oxopropyl)pyrrolidin-1-yl)-5-methyl-1-oxoheptan-4-yl)-N,3-dimethyl-2-((S)-3-methyl-2-(methylamino)butanamido)butanamide;
(S)-2-((S)-2-(dimethylamino)-3-methylbutanamido)-N-((3R,4S,5S)-3-methoxy-1-((S)-2-((R,2R)-1-methoxy-2-methyl-3-oxo-3-((2-(piperazin-1-yl)ethyl)amino)propyl)pyrrolidin-1-yl)-5-methyl-1-oxoheptan-4-yl)-N,3-dimethylbutanamide;
(S)—N-((3R,4S,5S)-3-methoxy-1-((S)-2-((1R,2R)-1-methoxy-2-methyl-3-oxo-3-((2-(piperazin-1-yl)ethyl)amino)propyl)pyrrolidin-1-yl)-5-methyl-1-oxoheptan-4-yl)-N,3-dimethyl-2-((S)-3-methyl-2-(methylamino)butanamido)butanamide;
(S)—N-((3R,4S,5S)-3-methoxy-1-((S)-2-((1R,2R)-1-methoxy-2-methyl-3-((2-(4-methylpiperazin-1-yl)ethyl)amino)-3-oxopropyl)pyrrolidin-1-yl)-5-methyl-1-oxoheptan-4-yl)-N,3-dimethyl-2-((S)-3-methyl-2-(methylamino)butanamido)butanamide;
4-amino-N—((S)-1-(((3R,4S,5S)-3-methoxy-1-((S)-2-((1R,2R)-1-methoxy-2-methyl-3-((2-morpholinoethyl)amino)-3-oxopropyl)pyrrolidin-1-yl)-5-methyl-1-oxoheptan-4-yl)(methyl)amino)-3-methyl-1-oxobutan-2-yl)benzamide;
4-amino-N—((S)-1-(((3R,4S,5S)-3-methoxy-1-((S)-2-((1R,2R)-1-methoxy-2-methyl-3-oxo-3-((2-(piperazin-1-yl)ethyl)amino)propyl)pyrrolidin-1-yl)-5-methyl-1-oxoheptan-4-yl)(methyl)amino)-3-methyl-1-oxobutan-2-yl)benzamide; and
(S)—N-((3R,4S,5S)-1-((S)-2-((1R,2R)-3-((4-aminophenethyl)amino)-1-methoxy-2-methyl-3-oxopropyl)pyrrolidin-1-yl)-3-methoxy-5-methyl-1-oxoheptan-4-yl)-2-((S)-2-(dimethylamino)-3-methylbutanamido)-N,3-dimethylbutanamide;
and pharmaceutically acceptable salts thereof.

In other embodiments, compounds of Formula (II) are conjugates comprising the species of Formula (I) described herein.

Compounds of Formula (I) will now be described by reference to illustrative synthetic schemes for their general preparation below and the specific examples that follow. Artisans will recognize that, to obtain the various compounds herein, starting materials may be suitably selected so that the ultimately desired substituents will be carried through the reaction scheme with or without protection as appropriate to yield the desired product. Alternatively, it may be necessary or desirable to employ, in the place of the ultimately desired substituent, a suitable group that may be carried through the reaction scheme and replaced as appropriate with the desired substituent. In addition, one of skill in the art will recognize that protecting groups may be used to protect certain functional groups (amino, carboxy, or sidechain groups) from reaction conditions, and that such groups are removed under standard conditions when appropriate. Each of the reactions depicted in Scheme A is preferably run at a temperature from about room temperature to the reflux temperature of the organic solvent used. Unless otherwise specified, the variables are as defined above in reference to Formula (I).

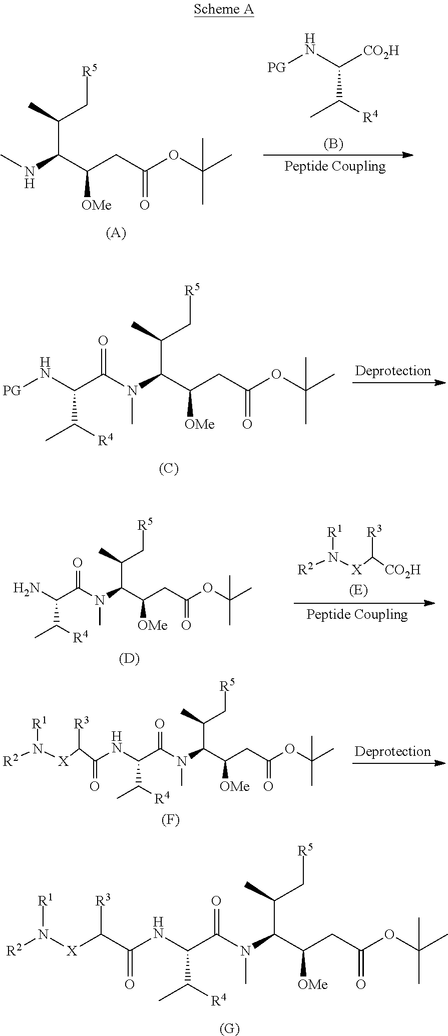

Referring to Scheme A, the preparation of compounds of Formula (I) begins with a protected acid form of dolaisoleucine (Dil) labeled (A) (see Pettit et al. (1994) J. Org. Chem. 59:1796-1800). Compound (A) is depicted with a tert-butyl ester protecting group, but one of skill in the art may select an appropriate replacement. Coupling with a nitrogen-protected valine or isoleucine derivative (B), where PG is a suitable amino protecting group such as a Boc (tert-butoxycarbonyl) or fluorenylmethyloxycarbonyl (Fmoc) group, is effected under standard peptide coupling conditions. For example, reactions are run in the presence of diethyl cyanophosphonate (DEPC), PyBrOP, PyBOP, BOP, diisopropylcarbodiimide (DIC), dicyclohexylcarbodiimide (DCC), 1-(3-dimethylaminopropyl)-3-ethylcarbodiimide hydrochloride (EDCI), 1-hydroxybenzotriazole (HOBt), 1-hydroxy-7-aza-benzotriazole (HOAt), HBTU (O-benzotriazol-1-yl-N,N,N′,N′-tetramethyluronium hexafluorophosphate), HATU (O-(7-azabenzotriazol-1-yl)-1,1,3,3-tetramethyluronium hexafluorophosphate), and the like, or a combination thereof. Reactions are typically run in the presence of a tertiary amine base, such as diisopropylethylamine. Suitable solvents include dichloromethane, N,N-dimethylformamide (DMF), dimethyl sulfoxide (DMSO), ethyl acetate and the like. The amino protecting group on resultant dipeptide (C) is removed by deprotection under suitable conditions. For example, where PG is a Boc group, compound (C) is treated with trifluoroacetic acid to form free amine (D). Where PG is an Fmoc group, compound (C) is treated with piperidine or diethylamine to yield compound (D). Compound (D) is then coupled to amino acid derivative (E), in protected form if necessary, under peptide coupling conditions as described above, to generate tripeptide (F). Treatment with acid removes the carboxy protecting group to provide free acid (G).

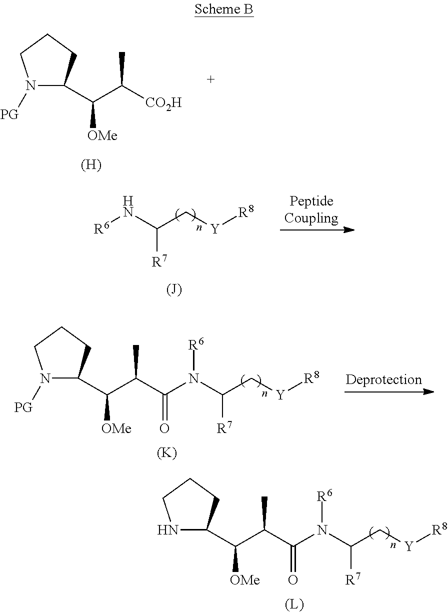

Referring to Scheme B, the amino-protected dolaproine (Dap) designated as (H) (see Pettit et al. (1994) J. Org. Chem. 59:6287-6295) is coupled with amine (J) (which is prepared using methods known to one in the art) under peptide coupling conditions as described above. Resulting dipeptide (K) is deprotected as discussed for Scheme A to provide compound (L).

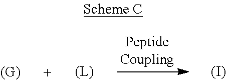

Referring to Scheme C, acid (G) and amine (L) are coupled under peptide coupling conditions as discussed above to provide compounds of Formula (I). Where the result of the reaction is a protected form of Formula (I), suitable deprotection conditions are employed to give the target compound.

The generation of antibody-drug conjugate compounds can be accomplished by any technique known to the skilled artisan. Briefly, the antibody-drug conjugate compounds comprise an antibody unit, a drug, and optionally a linker that joins the drug and the antibody. A number of different reactions are available for covalent attachment of drugs to linkers and linkers to antibodies to arrive at the conjugates. The coupling may be accomplished by reacting the drug with the linker, and then the drug-linker moiety to the antibody. Alternatively, coupling is accomplished by reacting the linker with the antibody and then attaching the drug to the pendant linker groups. In a further embodiment, the drug moiety is covalently attached directly to a suitable functional group on the antibody, such as an amino group, a thiol, a phenol, or an acid.

In certain embodiments, a linker intermediate, which is a precursor of the linker, is reacted with the drug under appropriate conditions. In certain embodiments, reactive groups are used on the drug and/or the intermediate. The product of the reaction between the drug and the intermediate, or the derivatized drug, is subsequently reacted with the antibody under appropriate conditions.

Suitable coupling reactions often involve reaction of the amino acid residues of the antibody molecule, including the amine groups of lysine, the free carboxylic acid groups of glutamic and aspartic acid, the sulfhydryl groups of cysteine and the various moieties of the aromatic amino acids, with a linker or linker-drug moiety. One of the most commonly used non-specific methods of covalent attachment is the carbodiimide reaction to link a carboxy (or amino) group of a drug or linker to amino (or carboxy) groups of the antibody. Additionally, bifunctional agents such as dialdehydes or imidoesters have been used to link the amino group of a drug or linker to amino groups of an antibody molecule. Also available for attachment of drugs to antibodies is the Schiff base reaction. This method involves the periodate oxidation of a drug that contains glycol or hydroxy groups, thus forming an aldehyde which is then reacted with the linker to form a Schiff base. Isothiocyanates can also be used as coupling agents for covalently attaching drugs to linkers. Similar reactions are used to couple the linkers to the antibody. In other embodiments, coupling of a linker, toxin-linker, or toxin to a tyrosine residue of the antibody may be accomplished (see, e.g., Ban et al. J. Am. Chem. Soc. 2010, 132, 1523-1525). Other techniques are known to the skilled artisan and within the scope of the present invention.

Conjugates of the antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene).

In one embodiment, the peptide analogs described herein are attached to the antibody through the N (amino) terminus or the C (carboxyl) terminus of the peptidic drug moiety (WO 02/088172).

The following examples are offered to illustrate but not to limit the invention. The compounds are prepared using the general methods described above.

### Example 1

### (S)—N-((3R,4S,5S)-3-Methoxy-1-((S)-2-((1R,2R)-1-methoxy-2-methyl-3-oxo-3-((2-(pyridin-2-yl)ethyl)amino)propyl)pyrrolidin-1-yl)-5-methyl-1-oxoheptan-4-yl)-N,3-dimethyl-2-((S)-3-methyl-2-(methylamino)butanamido)butanamide, TFA salt

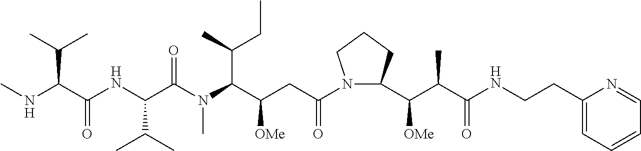 [SMILES: CC[C@H](C)[C@@H]([C@@H](CC(=O)N1CCC[C@H]1[C@H](OC)[C@@H](C)C(=O)NCCc1ccccn1)OC)N(C)C(=O)[C@@H](NC(=O)[C@@H](NC)C(C)C)C(C)C]

### Step 1. Preparation of Boc-Dap-2-(2-pyridyl)ethylamine

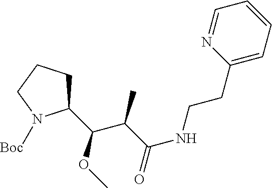 [SMILES: CO[C@H]([C@@H](C)C(=O)NCCc1ccccn1)[C@@H]1CCCN1C(=O)OC(C)(C)C]

To a stirred room temperature suspension of Boc-Dap-OH dicyclohexylamine salt (1.00 g, 2.13 mmol) and 2-(2-pyridyl)ethylamine (0.28 mL, 2.3 mmol) in CH₂Cl₂ (5 mL) was added diisopropylethylamine (DIEA; 0.760 mL, 4.26 mmol), followed by DEPC (0.483 mL, 3.20 mmol). After 8 hours, analysis by liquid chromatograph/mass spectrometry (LCMS) showed the reaction was complete. Boc-Dap-2-(2-pyridyl)ethylamine was isolated by flash chromatography on silica gel (silica gel 40 μm, 60 Å, 3.0×17.0 cm) using 2% to 10% MeOH/1% NEt₃ in CH₂Cl₂ as the eluent. A total of 0.80 g of Boc-Dap-2-(2-pyridyl)ethylamine (2.0 mmol, 95% yield) was obtained.

### Step 2. Preparation of Fmoc-Val-Dil-OtBu

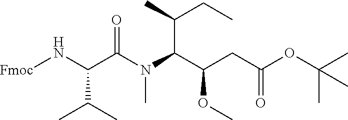

To a solution of Dil-OtBu hydrochloride (0.60 g, 2.03 mmol) and Fmoc-Val-OH (0.829 g, 2.44 mmol) stirring in ethyl acetate (EtOAc) (3 mL) was added DIEA (0.65 mL, 3.7 mmol). The reaction was cooled to 0° C. and stirred for 20 minutes, followed by the addition of DIEA (0.65 mL, 3.7 mmol). The reaction mixture was cooled for another 20 minutes, followed by the addition of 2-chloro-1-methylpyridinium iodide (CMPI; 0.83 g, 3.7 mmol). After 8 hours, analysis by LCMS showed the reaction was complete. The reaction mixture was washed with 1 M HCl (25 mL×2) and brine (50 mL). The organic phase was dried over magnesium sulfate, filtered, and concentrated in vacuo. Fmoc-Val-Dil-OtBu was isolated by flash chromatography on silica gel (silica gel 40 μm, 60 Å, 3.0×17.0 cm) using 18% to 90% EtOAc in Hexanes as the eluent. A total of 1.1 g of Fmoc-Val-Dil-OtBu (1.9 mmol, 93% yield) was obtained.

### Step 3. Preparation of Fmoc-Val-Dil-Dap-2-(2-pyridyl)ethylamine

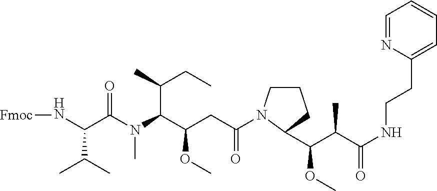

To a stirred room temperature suspension of Fmoc-Val-Dil-OtBu (0.883 g, 1.52 mmol) and Boc-Dap-2-(2-pyridyl)ethylamine (0.451 g, 1.52 mmol) in CH₂Cl₂ (10 mL) was added trifluoroacetic acid (TFA; 5 mL). After 8 hours, analysis by LCMS showed the reaction was complete. Volatile organics were evaporated in vacuo to yield crude Fmoc-Val-Dil-OH and H-Dap-2-(2-pyridyl)ethylamine TFA salt that were used without further purification.

To a stirred room temperature suspension of crude H-Dap-2-(2-pyridyl)ethylamine TFA salt and Fmoc-Val-Dil-OH in EtOAc (2 mL) was added DIEA (1.10 mL, 6.08 mmol), followed by DEPC (0.92 mL, 6.08 mmol). After 15 hours, analysis by LCMS showed the reaction was complete. The reaction was washed with a saturated NaHCO₃ solution (50 mL) followed by water (50 mL×2). The organic fraction was filtered through a pad of magnesium sulfate and concentrated in vacuo. The resulting viscous oil was purified by flash chromatography on silica gel (silica gel 40 μm, 60 Å, 23×123 mm) using 5% to 10% MeOH in CH₂Cl₂ as the eluent. A total of 0.888 g of Fmoc-Val-Dil-Dap-2-(2-pyridyl)ethylamine (1.11 mmol, 73% yield) was obtained.

### Step 4

To a stirred room temperature solution of Fmoc-Val-Dil-Dap-2-(2-pyridyl)ethylamine (1.75 g, 2.19 mmol) in CH₂Cl₂ (5 mL) was added piperidine (5 mL). After 8 hours, analysis by LCMS showed the reaction was complete. Volatile organics were evaporated in vacuo to yield crude H-Val-Dil-Dap-2-(2-pyridyl)ethylamine that was used without further purification.

To a stirred room temperature suspension of crude H-Val-Dil-Dap-2-(2-pyridyl)ethylamine and Fmoc-MeVal-OH (1.55 g, 4.38 mmol) in CH₂Cl₂ (7 mL) was added DIEA (1.56 mL, 8.76 mmol), followed by DEPC (1.32 mL, 8.76 mmol). After 18 hours, analysis by LCMS showed the reaction was complete. The solvent was removed in vacuo, and the crude mixture was dissolved in EtOAc (100 mL) and washed with a saturated NaHCO₃ solution (50 mL), and water (50 mL×2). The organic fraction was filtered through a pad of magnesium sulfate and concentrated in vacuo. The resulting oil was purified by flash on silica gel (silica gel 40 μm, 60 Å 23×123 mm) using 5% to 10% MeOH in CH₂Cl₂ as the eluent. A total of 1.07 g of Fmoc-MeVal-Val-Dil-Dap-2-(2-pyridyl)ethylamine (1.17 mmol, 59% yield) was obtained.

To a stirred room temperature solution of Fmoc-MeVal-Val-Dil-Dap-2-(2-pyridyl)ethylamine (1.07 g, 1.17 mmol) in CH₂Cl₂ (5 mL) was added piperidine (5 mL). After 10 hours, analysis by LCMS showed the reaction was complete. The product was purified by preparatory reverse phase high performance liquid chromatograph (RP-HPLC), using a Phenomenex Synergi 10μ Max-RP 80 Å column (150×30.00 mm) using 10% to 100% acetonitrile in 0.05% aqueous TFA solution. A total of 0.624 g (64% yield) of the title compound was obtained as the TFA salt. LCMS (all LCMS data was acquired on Aquity UPLC BeH C8 1.7 μm 2.1×50 mm column, 40° C.; 0-0.5 min: isocratic 90 water/10 acetonitrile/0.05 TFA; 0-4 min: linear gradient 90 water/10 acetonitrile/0.05 TFA to 10 water/90 acetonitrile/0.05 TFA), retention time (RT)=1.89 minutes; ESI-MS m/z 689.76 [M+H]⁺; 711.63 [M+Na]⁺; UV λₘₐₓ 215, 264; HRMS m/z 689.4966 [C₃₇H₆₄N₆O₆+H]⁺.

### Example 2

### 4-Amino-N—((S)-1-(((3R,4S,5S)-3-methoxy-1-((S)-2-((1R,2R)-1-methoxy-2-methyl-3-oxo-3-((2-(pyridin-2-yl)ethyl)amino)propyl)pyrrolidin-1-yl)-5-methyl-1-oxoheptan-4-yl)(methyl)amino)-3-methyl-1-oxobutan-2-yl)benzamide, TFA salt

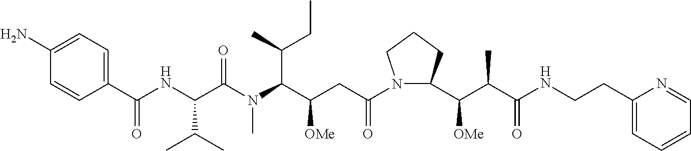 [SMILES: CC[C@H](C)[C@@H]([C@@H](CC(=O)N1CCC[C@H]1[C@H](OC)[C@@H](C)C(=O)NCCc1ccccn1)OC)N(C)C(=O)[C@@H](NC(=O)c1ccc(N)cc1)C(C)C]

To stirred room temperature solution of Fmoc-Val-Dil-Dap-2(2-pyridyl)ethylamine (113 mg, 0.141 mmol) in CH₂Cl₂ (5 mL) was added piperidine (2 mL). After 8 hours, analysis by LCMS showed the reaction was complete. Volatile organics were evaporated in vacuo to yield crude H-Val-Dil-Dap-2-(2-pyridyl)ethylamine that was used without further purification.

To stirred room temperature suspension of crude H-Val-Dil-Dap-2-(2-pyridyl)ethylamine and N-Boc-4-Abz-OH (40 mg, 0.16 mmol) in CH₂Cl₂ (3 mL) was added DIEA (0.10 mL, 0.56 mmol), followed by DEPC (0.08 mL, 0.56 mmol). After 18 hours, analysis by LCMS showed the reaction was complete. The solvent was removed in vacuo and the crude product was dissolved in EtOAc (10 mL) and washed with a saturated NaHCO₃ solution (5 mL), and water (5 mL×2). The organic fraction was filtered through a pad of magnesium sulfate and concentrated in vacuo. The resulting viscous oil was purified by preparatory RP-HPLC, using a Phenomenex Synergi 10μ Max-RP 80 Å column (150×30.00 mm) using 10% to 100% acetonitrile in 0.05% aqueous TFA solution. A total of 60 mg (45% yield) of N-Boc-4-Abz-Val-Dil-Dap-2-(2-pyridyl)ethylamine TFA salt was obtained.

To a stirred room temperature solution of N-Boc-4-Abz-Val-Dil-Dap-2-(2-pyridyl)ethylamine TFA salt (20 mg, 0.021 mmol) in CH₂Cl₂ (0.5 mL) was added TFA (0.5 mL). After 10 hours, analysis by LCMS showed the reaction was complete. The product was purified by preparatory RP-HPLC, using a Phenomenex Synergi 10μ Max-RP 80 Å column (150×30.00 mm) using 10% to 100% acetonitrile in a 0.05% aqueous TFA solution. A total of 16 mg (91% yield) of the title compound was obtained as the TFA salt. LCMS RT=2.66 minutes; ESI-MS m/z 695.51 [M+H]⁺; 717.46 [M+Na]⁺; UV λₘₐₓ 215, 266; HRMS m/z 695.4496 [C₃₈H₅₈N₆O₆+H]⁺.

### Example 3

### (S)—N-((3R,4S,5S)-3-Methoxy-1-((S)-2-((1R,2R)-1-methoxy-2-methyl-3-oxo-3-((2-(pyridin-3-yl)ethyl)amino)propyl)pyrrolidin-1-yl)-5-methyl-1-oxoheptan-4-yl)-N,3-dimethyl-2-((S)-3-methyl-2-(methylamino)butanamido)butanamide, TFA salt

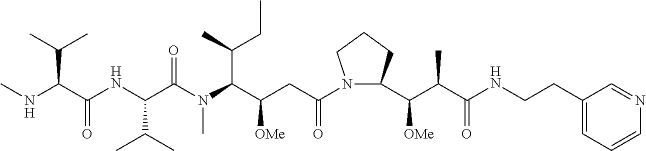 [SMILES: CC[C@H](C)[C@@H]([C@@H](CC(=O)N1CCC[C@H]1[C@H](OC)[C@@H](C)C(=O)NCCc1cccnc1)OC)N(C)C(=O)[C@@H](NC(=O)[C@@H](NC)C(C)C)C(C)C]

### Step 1. Preparation of Boc-Dap-3-(2-aminoethyl)pyridine

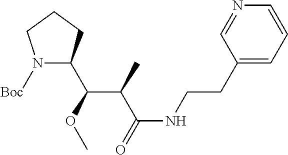 [SMILES: CO[C@H]([C@@H](C)C(=O)NCCc1cccnc1)[C@@H]1CCCN1C(=O)OC(C)(C)C]

To a stirred room temperature suspension of Boc-Dap-OH dicyclohexylamine salt (1.65 g, 3.52 mmol) and 3-(2-aminoethyl)pyridine dihydrobromide (1.00 g, 3.52 mmol) in CH₂Cl₂ (10 mL) was added DIEA (1.26 mL, 7.04 mmol), followed by DEPC (0.80 mL, 5.28 mmol). After 8 hours, analysis by LCMS showed the reaction was complete. Boc-Dap-3-(2-aminoethyl)pyridine was isolated by flash chromatography on silica gel (silica gel 40 μm, 60 Å, 9.0×20.0 cm) using 2% to 10% MeOH/1% NEt₃ in CH₂Cl₂ as the eluent. A total of 1.29 g (3.30 mmol, 94% yield) of Boc-Dap-3-(2-aminoethyl)pyridine was obtained.

### Step 2. Preparation of Fmoc-Val-Dil-Dap-3-(2-aminoethyl)pyridine

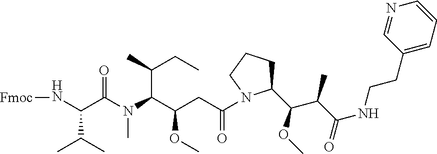

To a stirred room temperature suspension of Fmoc-Val-Dil-OtBu (3.53 g, 6.08 mmol) and Boc-Dap-3-(2-aminoethyl)pyridine (2.38 g, 6.08 mmol) in CH₂Cl₂ (20 mL) was added TFA (10 mL). After 8 hours, analysis by LCMS showed the reaction was complete. Volatile organics were evaporated in vacuo to yield crude Fmoc-Val-Dil-OH and H-Dap-3-(2-aminoethyl)pyridine TFA salt that were used without further purification.

To a stirred room temperature suspension of crude H-Dap-3-(2-aminoethyl)pyridine TFA salt and Fmoc-Val-Dil-OH in EtOAc (15 mL) was added DIEA (4.34 mL, 24.3 mmol), followed by DEPC (3.67 mL, 24.3 mmol). After 15 hours, analysis by LCMS showed the reaction was complete. The reaction was washed with a saturated NaHCO₃ solution (150 mL) followed by water (150 mL×2). The organic fraction was filtered through a pad of magnesium sulfate and concentrated in vacuo. The resulting viscous oil was dissolved in a minimal amount of CH₂Cl₂ and purified by flash chromatography on silica gel (silica gel 40 m, 60 Å, 3.0×17.0 cm using 5% to 10% MeOH in CH₂Cl₂ as the eluent. A total of 3.65 g of Fmoc-Val-Dil-Dap-3-(2-aminoethyl)pyridine (4.57 mmol, 75% yield) was obtained.

### Step 3

To stirred room temperature solution of Fmoc-Val-Dil-Dap-3-(2-aminoethyl)pyridine (3.65 g, 4.57 mmol) in CH₂Cl₂ (10 mL) was added piperidine (3 mL). After 8 hours, analysis by LCMS showed the reaction was complete. Volatile organics were evaporated in vacuo, to yield crude H-Val-Dil-Dap-3-(2-aminoethyl)pyridine that was used without further purification.

To a stirred room temperature suspension of crude H-Val-Dil-Dap-3-(2-aminoethyl)pyridine and Fmoc-MeVal-OH (3.23 g, 9.14 mmol) in CH₂Cl₂ (15 mL) was added DIEA (3.26 mL, 18.3 mmol), followed by DEPC (2.76 mL, 18.3 mmol). After 18 hours, analysis by LCMS showed the reaction was complete. The solvent was removed in vacuo, the crude product was dissolved in EtOAc (100 mL) and washed with a saturated NaHCO₃ solution (150 mL), and water (150 mL×2). The organic fraction was filtered through a pad of magnesium sulfate and concentrated in vacuo. The resulting viscous oil was purified by flash chromatography on silica gel (silica gel 40 m, 60 Å, 3.0×17.0 cm) using 5% to 10% MeOH in CH₂Cl₂ as the eluent. A total of 2.41 g of Fmoc-MeVal-Val-Dil-Dap-3-(2-aminoethyl)pyridine (2.64 mmol, 58% yield) was obtained.

To a stirred room temperature solution of Fmoc-MeVal-Val-Dil-Dap-3-(2-aminoethyl)pyridine (2.41 g, 2.64 mmol) in CH₂Cl₂ (10 mL) was added piperidine (3 mL). After hours, analysis by LCMS showed the reaction was complete. The product was purified by preparatory RP-HPLC, using a Phenomenex Synergi 10V Max-RP 80 Å column (150×30.00 mm) using 10% to 100% acetonitrile in a 0.05% aqueous TFA solution as the eluent. A total of 1.32 g (1.64 mmol, 62% yield) of the title compound was obtained as the TFA salt. LCMS RT=1.88 minutes; ESI-MS m/z 689.55 [M+H]⁺; 711.62 [M+Na]⁺; UV λₘₐₓ 215, 261; HRMS m/z 689.4957 [C₃₇H₆₄N₆O₆+H]⁺.

### Example 4

### 4-Amino-N—((S)-1-(((3R,4S,5S)-3-methoxy-1-((S)-2-((1R,2R)-1-methoxy-2-methyl-3-oxo-3-((2-(pyridin-3-yl)ethyl)amino)propyl)pyrrolidin-1-yl)-5-methyl-1-oxoheptan-4-yl)(methyl)amino)-3-methyl-1-oxobutan-2-yl)benzamide, TFA salt

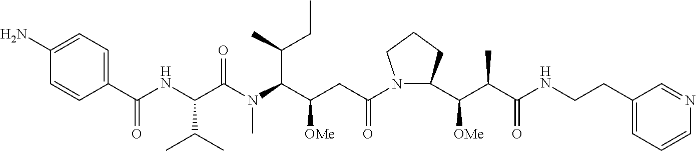 [SMILES: CC[C@H](C)[C@@H]([C@@H](CC(=O)N1CCC[C@H]1[C@H](OC)[C@@H](C)C(=O)NCCc1cccnc1)OC)N(C)C(=O)[C@@H](NC(=O)c1ccc(N)cc1)C(C)C]

To a stirred room temperature solution of Fmoc-Val-Dil-Dap-3-(2-aminoethyl)pyridine (1.59 g, 1.99 mmol) in CH₂Cl₂ (5 mL) was added piperidine (2 mL). After 8 hours, analysis by LCMS showed the reaction was complete. Volatile organics were evaporated in vacuo, to yield crude H-Val-Dil-Dap-3-(2-aminoethyl)pyridine that was used without further purification.

To a stirred room temperature suspension of crude H-Val-Dil-Dap-3-(2-aminoethyl)pyridine and N-Boc-4-Abz-OH (0.94 g, 3.98 mmol) in CH₂Cl₂ (5 mL) was added DIEA (1.42 mL, 7.96 mmol), followed by DEPC (1.20 mL, 7.96 mmol). After 18 hours, analysis by LCMS showed the reaction was complete. The solvent was removed in vacuo, the crude product was dissolved in EtOAc (20 mL) and washed with a saturated NaHCO₃ solution (50 mL), and water (50 mL×2). The organic fraction was filtered through a pad of magnesium sulfate and concentrated in vacuo. The resulting viscous oil was purified by flash chromatography on silica gel (silica gel 40 μm, 60 Å, 3.0×17.0 cm) using 5% to 10% MeOH in CH₂Cl₂ as the eluent. A total of 1.11 g of N-Boc-4-Abz-Val-Dil-Dap-3-(2-aminoethyl)pyridine (1.40 mmol, 70% yield) was obtained.

To a stirred room temperature solution of N-Boc-4-Abz-Val-Dil-Dap-3-(2-aminoethyl)pyridine (1.11 g, 1.40 mmol) in CH₂Cl₂ (10 mL) was added TFA (2 mL). After 10 hours, analysis by LCMS showed the reaction was complete. The product was purified by preparatory RP-HPLC, using a Phenomenex Synergi 10μ Max-RP 80 Å column (150×30.00 mm) using 10% to 100% acetonitrile in a aqueous 0.05% TFA solution as the eluent. A total of 0.684 g of the title compound was obtained as the TFA salt (0.815 mmol, 58% yield). LCMS RT=2.04 minutes; ESI-MS m/z 695.56 [M+H]⁺; 717.47 [M+Na]⁺; UV λₘₐₓ 215, 264; HRMS m/z 695.4496 [C₃₈H₅₈N₆O₆+H]⁺.

### Example 5

### (S)—N-((3R,4S,5S)-3-Methoxy-1-((S)-2-((1R,2R)-1-methoxy-2-methyl-3-oxo-3-((2-(pyridin-4-yl)ethyl)amino)propyl)pyrrolidin-1-yl)-5-methyl-1-oxoheptan-4-yl)-N,3-dimethyl-2-((S)-3-methyl-2-(methylamino)butanamido)butanamide, TFA salt

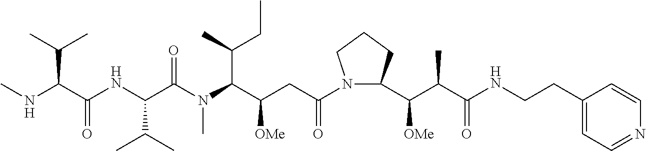 [SMILES: CC[C@H](C)[C@@H]([C@@H](CC(=O)N1CCC[C@H]1[C@H](OC)[C@@H](C)C(=O)NCCc1ccncc1)OC)N(C)C(=O)[C@@H](NC(=O)[C@@H](NC)C(C)C)C(C)C]

### Step 1. Preparation of Boc-Dap-4-(2-aminoethyl)pyridine

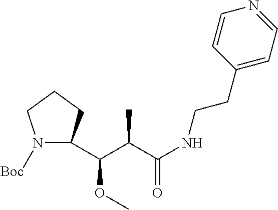 [SMILES: CO[C@H]([C@@H](C)C(=O)NCCc1ccncc1)[C@@H]1CCCN1C(=O)OC(C)(C)C]

To a stirred room temperature suspension of Boc-Dap-OH dicyclohexylamine salt (3.84 g, 8.19 mmol) and 4-(2-aminoethyl)pyridine (0.977 mL, 8.19 mmol) in CH₂Cl₂ (10 mL) was added DIEA (2.92 mL, 16.4 mmol), followed by DEPC (1.85 mL, 12.3 mmol). After 8 hours, analysis by LCMS showed the reaction was complete. Boc-Dap-4-(2-aminoethyl)pyridine was isolated by flash chromatography on silica gel (silica gel 40 μm, 60 Å, 9.0×20.0 cm) using 2% to 10% MeOH/1% NEt₃ in CH₂Cl₂ as the eluent. A total of 2.92 g of Boc-Dap-4-(2-aminoethyl)pyridine (7.46 mmol, 91% yield) was obtained.

### Step 2. Preparation of Fmoc-Val-Dil-Dap-4-(2-aminoethyl)pyridine

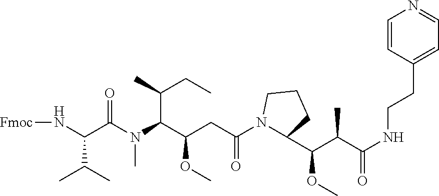

To stirred a room temperature suspension of Fmoc-Val-Dil-OtBu (4.33 g, 7.46 mmol) and Boc-Dap-4-(2-aminoethyl)pyridine (2.92 g, 7.46 mmol) in CH₂Cl₂ (25 mL) was added TFA (15 mL). After 8 hours, analysis by LCMS showed the reaction was complete. Volatile organics were evaporated in vacuo, to yield crude Fmoc-Val-Dil-OH and H-Dap-4-(2-aminoethyl)pyridine TFA salt that were used without further purification.

To a stirred room temperature suspension of crude H-Dap-4-(2-aminoethyl)pyridine TFA salt and Fmoc-Val-Dil-OH in EtOAc (20 mL) was added DIEA (5.32 mL, 29.8 mmol), followed by DEPC (4.50 mL, 29.8 mmol). After 15 hours, analysis by LCMS showed the reaction was complete. The reaction was washed with a saturated NaHCO₃ solution (150 mL) followed by water (150 mL×2). The organic fraction was filtered through a pad of magnesium sulfate and concentrated in vacuo. The resulting viscous oil was dissolved in a minimal amount of CH₂Cl₂ and purified by flash chromatography on silica gel (silica gel 40 m, 60 Å, 3.0×17.0 cm) using 5% to 10% MeOH in CH₂Cl₂ as the eluent. A total of 2.51 g of Fmoc-Val-Dil-Dap-4-(2-aminoethyl)pyridine (3.15 mmol, 53% yield) was obtained.

### Step 3

To a stirred room temperature solution of Fmoc-Val-Dil-Dap-4-(2-aminoethyl)pyridine (5.74 g, 7.19 mmol) in CH₂Cl₂ (5 mL) was added piperidine (5 mL). After 8 hours, analysis by LCMS showed the reaction was complete. Volatile organics were evaporated in vacuo to yield crude H-Val-Dil-Dap-4-(2-aminoethyl)pyridine that was used without further purification.

To a stirred room temperature suspension of crude H-Val-Dil-Dap-4-(2-aminoethyl)pyridine and Fmoc-MeVal-OH (5.08 g, 14.4 mmol) in CH₂Cl₂ (15 mL) was added DIEA (5.13 mL, 28.8 mmol), followed by DEPC (4.34 mL, 28.8 mmol). After 18 hours, analysis by LCMS showed the reaction was complete. The solvent was removed in vacuo, the crude product was dissolved in EtOAc (20 mL) and washed with a saturated NaHCO₃ solution (150 mL), and water (150 mL×2). The organic fraction was filtered through a pad of magnesium sulfate and concentrated in vacuo. The resulting viscous oil was purified by flash chromatography on silica gel (silica gel 40 m, 60 Å, 3.0×17.0 cm) using 5% to 10% MeOH in CH₂Cl₂ as the eluent. A total of 1.95 g of pure Fmoc-MeVal-Val-Dil-Dap-4-(2-aminoethyl)pyridine (2.15 mmol, 30% yield) was obtained.

To a stirred room temperature solution of Fmoc-MeVal-Val-Dil-Dap-4-(2-aminoethyl)pyridine (1.95 g, 2.15 mmol) in CH₂Cl₂ (5 mL) was added piperidine (2 mL). After hours, analysis by LCMS showed the reaction was complete. The product was purified by preparatory RP-HPLC, using a Phenomenex Synergi 10μ Max-RP 80 Å column (150×30.00 mm) using 10% to 100% acetonitrile in a 0.05% aqueous TFA solution as the eluent. A total of 0.963 g of the title compound was obtained as the TFA salt (1.16 mmol, 54% yield). LCMS RT=1.87 minutes; ESI-MS m/z 689.50 [M+H]⁺; 711.44 [M+Na]⁺; UV λₘₐₓ 218, 253; HRMS m/z 689.4966 [C₃₇H₆₄N₆O₆+H]⁺.

### Example 6

### 4-Amino-N—((S)-1-(((3R,4S,5S)-3-methoxy-1-((S)-2-((1R,2R)-1-methoxy-2-methyl-3-oxo-3-((2-(pyridin-4-yl)ethyl)amino)propyl)pyrrolidin-1-yl)-5-methyl-1-oxoheptan-4-yl)(methyl)amino)-3-methyl-1-oxobutan-2-yl)benzamide, TFA salt

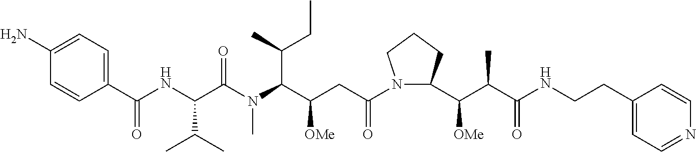 [SMILES: CC[C@H](C)[C@@H]([C@@H](CC(=O)N1CCC[C@H]1[C@H](OC)[C@@H](C)C(=O)NCCc1ccncc1)OC)N(C)C(=O)[C@@H](NC(=O)c1ccc(N)cc1)C(C)C]

To a stirred room temperature solution of Fmoc-Val-Dil-Dap-4-(2-aminoethyl)pyridine (3.46 g, 4.34 mmol) in CH₂Cl₂ (5 mL) was added piperidine (2 mL). After 8 hours, analysis by LCMS showed the reaction was complete. Volatile organics were evaporated in vacuo to yield crude H-Val-Dil-Dap-4-(2-aminoethyl)pyridine that was used without further purification.

To a stirred room temperature suspension of crude H-Val-Dil-Dap-4-(2-aminoethyl)pyridine and N-Boc-4-Abz-OH (2.06 g, 8.68 mmol) in CH₂Cl₂ (7 mL) was added DIEA (3.10 mL, 17.4 mmol), followed by DEPC (2.62 mL, 17.4 mmol). After 18 hours, analysis by LCMS showed the reaction was complete. The solvent was removed in vacuo, the crude product was dissolved in EtOAc (20 mL) and washed with saturated NaHCO₃ solution (50 mL), and water (50 mL×2). The organic fraction was filtered through a pad of magnesium sulfate and concentrated in vacuo. The resulting viscous oil was purified by flash chromatography on silica gel (silica gel 40 m, 60 Å, 3.0×17.0 cm) using a 5% to 10% MeOH in CH₂Cl₂ as the eluent. A total of 2.09 g of N-Boc-4-Abz-Val-Dil-Dap-4-(2-aminoethyl)pyridine (2.63 mmol, 61% yield) was obtained.

To a stirred room temperature solution of Boc-4-Abz-Val-Dil-Dap-4-(2-aminoethyl)pyridine (2.09 g, 2.63 mmol) in CH₂Cl₂ (10 mL) was added TFA (2 mL). After 10 hours, analysis by LCMS showed the reaction was complete. The product was purified by preparatory RP-HPLC, using a Phenomenex Synergi 10μ Max-RP 80 Å column (150×30.00 mm) using 10% to 100% acetonitrile in a aqueous 0.05% TFA solution as the eluent. A total of 0.633 g of the title compound was obtained as the TFA salt (0.755 mmol, 29% yield). LCMS RT=2.08 minutes; ESI-MS m/z 695.53 [M+H]⁺; 717.45 [M+Na]⁺; UV λₘₐₓ 212, 254; HRMS m/z 695.4496 [C₃₈H₅₈N₆O₆+H]⁺.

### Example 7

### (S)—N-((3R,4S,5S)-1-((S)-2-((1R,2R)-3-((2-(1H-Imidazol-1-yl)ethyl)amino)-1-methoxy-2-methyl-3-oxopropyl)pyrrolidin-1-yl)-3-methoxy-5-methyl-1-oxoheptan-4-yl)-N,3-dimethyl-2-((S)-3-methyl-2-(methylamino)butanamido)butanamide, TFA salt

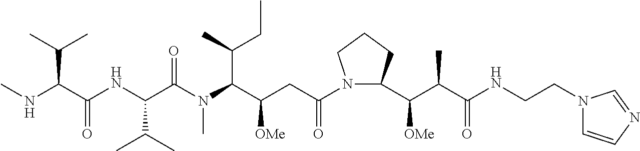 [SMILES: CC[C@H](C)[C@@H]([C@@H](CC(=O)N1CCC[C@H]1[C@H](OC)[C@@H](C)C(=O)NCCn1ccnc1)OC)N(C)C(=O)[C@@H](NC(=O)[C@@H](NC)C(C)C)C(C)C]

### Step 1. Preparation of Fmoc-MeVal-Val-Dil-OtBu

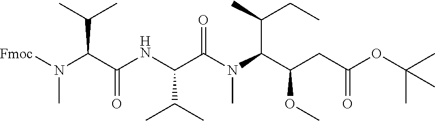

To a stirred room temperature solution of Fmoc-Val-Dil-OtBu (5.00 g, 8.61 mmol) in CH₂Cl₂ (10 mL) was added piperidine (5 mL). After 8 hours, analysis by LCMS showed the reaction was complete. Volatile organics were evaporated in vacuo to yield crude H-Val-Dil-OtBu that was used without further purification.

To a stirred room temperature suspension of crude H-Val-Dil-OtBu and Fmoc-MeVal-OH (9.13 g, 25.8 mmol) in EtOAc (10 mL) was added DIEA (6.14 mL, 34.4 mmol), followed by DEPC (5.19 mL, 34.4 mmol). After 12 hours, analysis by LCMS showed the reaction was complete. The reaction mixture was washed with a 1 M HCl solution (150 mL×2) followed by brine (150 mL). The organic fraction was filtered through a pad of magnesium sulfate and concentrated in vacuo. The resulting viscous oil was purified by flash chromatography on silica gel (silica gel 40 m, 60 Å, 9.0×20.0 cm) using 18% to 90% EtOAc in hexanes as the eluent. A total of 5.54 g of Fmoc-MeVal-Val-Dil-OtBu (7.98 mmol, 93% yield) was obtained.

Step 2. Preparation of Boc-Dap-2-(1H-imidazole-1-yl)-ethylamine.

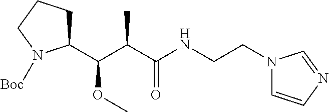 [SMILES: CO[C@H]([C@@H](C)C(=O)NCCn1ccnc1)[C@@H]1CCCN1C(=O)OC(C)(C)C]

To a stirred room temperature suspension of Boc-Dap-OH dicyclohexylamine salt (2.55 g, 5.43 mmol) and 2-(1H-imidazole-1-yl)-ethylamine dihydrochloride (1.00 g, 5.43 mmol) in CH₂Cl₂ (5 mL) was added DIEA (4.48 mL, 27.2 mmol), followed by DEPC (1.23 mL, 8.15 mmol). After 10 hours, analysis by LCMS showed the reaction was complete. Boc-Dap-2-(1H-imidazole-1-yl)-ethylamine was isolated by flash chromatography on silica gel (silica gel 40 m, 60 Å, 3.0×17.0 cm) using 2% to 10% MeOH/1% NEt₃ in CH₂Cl₂ as the eluent. A total of 1.64 g of Boc-Dap-2-(1H-imidazole-1-yl)-ethylamine (4.31 mmol, 79% yield) was obtained.

### Step 3

Fmoc-MeVal-Val-Dil-OtBu (5.47 g, 7.88 mmol) and Boc-Dap-2-(1H-imidazole-1-yl)-ethylamine (3.00 g, 7.88 mmol) were suspended in CH₂Cl₂ (10 mL), and to that stirring mixture was added TFA (10 mL). After 8 hours, analysis by LCMS showed the reaction was complete. The volatile organics were evaporated in vacuo to yield crude Fmoc-MeVal-Val-Dil-OH and H-Dap-2-(1H-imidazole-1-yl)-ethylamine TFA salt, which were used without further purification.

To a stirred room temperature suspension of crude Fmoc-MeVal-Val-Dil-OH and H-Dap-2-(1H-imidazole-1-yl)-ethylamine TFA salt in DMF (10 mL) was added DIEA (5.62 mL, 31.5 mmol), followed by HATU (5.99 g, 15.8 mmol) and HOBT (2.41 g, 15.8 mmol). After 12 hours, analysis by LCMS showed the reaction was complete. The reaction was terminated by addition of a saturated NaHCO₃ solution (20 mL), followed by extraction with EtOAc (4×20 mL). The combined organic extract was washed with brine (50 mL), dried over magnesium sulfate, filtered, and concentrated in vacuo. The resulting viscous oil was purified by preparatory RP-HPLC, using a Phenomenex Synergi 10μ Max-RP 80 Å column (150×30.00 mm) using 10% to 100% acetonitrile in a 0.05% aqueous TFA solution as the eluent. A total of 211 mg of Fmoc-MeVal-Val-Dil-Dap-2-(1H-imidazole-1-yl)-ethylamine TFA salt (0.202 mmol, 3% yield) was obtained.

To a stirred room temperature suspension of Fmoc-MeVal-Val-Dil-Dap-2-(1H-imidazole-1-yl)-ethylamine TFA salt (211 mg, 0.202 mmol) in CH₂Cl₂ (5 mL) was added piperidine (2 mL). After 10 hours, analysis by LCMS showed the reaction was complete. The product was purified by preparatory RP-HPLC, using a Phenomenex Synergi 10μ Max-RP 80 Å column (150×30.00 mm) using 10% to 100% acetonitrile in a 0.05% aqueous TFA solution as the eluent. A total of 100 mg of the title compound was obtained as the TFA salt (0.122 mmol, 60% yield). LCMS RT=1.95 minutes; ESI-MS m/z 678.50 [M+H]⁺; HRMS m/z 678.4918 [C₃₅H₆₃N₇O₆+H]⁺.

### Example 8

### N—((S)-1-(((3R,4S,5S)-1-((S)-2-((1R,2R)-3-((2-(1H-Imidazol-1-yl)ethyl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)-4-aminobenzamide, TFA salt

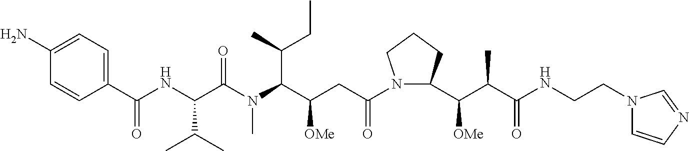 [SMILES: CC[C@H](C)[C@@H]([C@@H](CC(=O)N1CCC[C@H]1[C@H](OC)[C@@H](C)C(=O)NCCn1ccnc1)OC)N(C)C(=O)[C@@H](NC(=O)c1ccc(N)cc1)C(C)C]

### Step 1. Preparation of 4-Abz-Val-Dil-OtBu

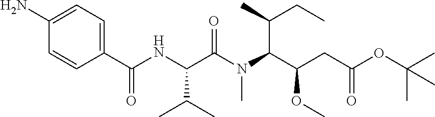 [SMILES: CC[C@H](C)[C@@H]([C@@H](CC(=O)OC(C)(C)C)OC)N(C)C(=O)[C@@H](NC(=O)c1ccc(N)cc1)C(C)C]

To a stirred room temperature solution of Fmoc-Val-Dil-OtBu (5.29 g, 9.11 mmol) in CH₂Cl₂ (10 mL) was added piperidine (5 mL). After 8 hours, analysis by LCMS showed the reaction was complete. Volatile organics were evaporated in vacuo to yield crude H-Val-Dil-OtBu that was used without further purification.

To a stirred room temperature suspension of H-Val-Dil-OtBu and Fmoc-4-Abz-OH (9.82 g, 27.3 mmol) in EtOAc (10 mL) was added DIEA (6.50 mL, 36.4 mmol), followed by DEPC (5.50 mL, 36.4 mmol). After 12 hours, analysis by LCMS showed the reaction was complete (including Fmoc deprotection). The reaction mixture was washed with water (250 mL×2), and brine (250 mL). The organic fraction was filtered through a pad of magnesium sulfate and concentrated in vacuo. The resulting oil was dissolved in CH₂Cl₂ (10 mL) and purified by flash chromatography on silica gel (silica gel 40 μm, 60 Å, 9.0×20.0 cm) using 18% to 90% EtOAc in Hexanes as the eluent. A total of 4.31 g of 4-Abz-Val-Dil-OtBu (9.02 mmol, 99% yield) was obtained.

### Step 2. Preparation of Cbz-4-Abz-Val-Dil-OtBu

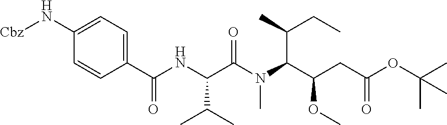 [SMILES: CC[C@H](C)[C@@H]([C@@H](CC(=O)OC(C)(C)C)OC)N(C)C(=O)[C@@H](NC(=O)c1ccc(NC(=O)OCc2ccccc2)cc1)C(C)C]

To a stirred room temperature suspension of H-Val-Dil-OtBu (3.52 g, 9.82 mmol) and Cbz-4-Abz-OH (4.00 g, 14.7 mmol) in CH₂Cl₂(20 mL) was added DIEA (7.01 mL, 39.3 mmol), followed by DEPC (5.93 mL, 39.3 mmol). After 18 hours, analysis by LCMS showed the reaction was complete. The reaction mixture was washed with water (250 mL×2), and brine (250 mL). The organic extract was filtered through a pad of magnesium sulfate and evaporated in vacuo. The resulting oil was purified by flash chromatography using on silica gel (silica gel μm, 60 Å, 9.0×20.0 cm) using 18% to 90% EtOAc in hexanes as the eluent. A total of 2.56 g of Cbz-4-Abz-Val-Dil-OtBu (4.18 mmol, 43% yield) was obtained.

### Step 3

Cbz-4-Abz-Val-Dil-OtBu (1.21 g, 1.97 mmol) and Boc-Dap-2-(1H-imidazole-1-yl)-ethylamine (0.75 g, 1.97 mmol) were suspended in CH₂Cl₂ (5 mL), and to the stirring solution was added TFA (5 mL). After 10 hours, analysis by LCMS showed the reaction was complete. The volatile organics were evaporated in vacuo to yield crude Cbz-4-Abz-Val-Dil-OH and H-Dap-2-(1H-imidazole-1-yl)-ethylamine TFA salt, which were used without further purification.

To a stirred room temperature suspension of crude Cbz-4-Abz-Val-Dil-OH and H-Dap-2-(1H-imidazole-1-yl)-ethylamine TFA salt in DMF (10 mL) was added DIEA (1.05 mL, 5.91 mmol), followed by HATU (1.50 g, 3.94 mmol) and HOBT (0.60 g, 3.94 mmol). After 8 hours, analysis by LCMS showed the reaction was complete. The reaction was terminated by the addition of a saturated NaHCO₃ solution (20 mL), followed by extraction with EtOAc (4×20 mL). The combined organic extract was washed with brine (50 mL), dried over a pad of magnesium sulfate, filtered, and concentrated in vacuo. The resulting viscous oil was purified by preparatory RP-HPLC, using a Phenomenex Synergi 10μ Max-RP 80 Å column (150×30.00 mm) using 10% to 100% acetonitrile in a 0.05% aqueous TFA solution as the eluent. A total of 0.654 g of Cbz-4-Abz-Val-Dil-Dap-2-(1H-imidazole-1-yl)-ethylamine TFA salt (0.68 mmol, 35% yield) was obtained.

To a stirred room temperature suspension of Cbz-4-Abz-Val-Dil-Dap-2-(1H-imidazole-1-yl)-ethylamine TFA salt (0.654 g, 0.68 mmol) and HCO₂NH₄ (8 mg, 1.20 mmol) in DMF (4 mL) and water (0.5 mL) was added 10% Pd/C (327 mg). After 8 hours, analysis by LCMS showed the reaction was complete. The reaction mixture was filtered through a pad of a diatomaceous earth and the filtrate was concentrated to give a total of 431 mg of the crude title compound as the formate salt (0.58 mmol, 85% yield). The product was purified by preparatory RP-HPLC, using a Phenomenex Synergi 10μ Max-RP 80 Å column (150×30.00 mm) using 10% to 100% acetonitrile in a 0.05% aqueous TFA solution as the eluent. A total of 367 mg of the title compound was obtained as the TFA salt (0.46 mmol, 79% yield). LCMS RT=2.17 minutes; ESI-MS m/z 684.68 [M+H]⁺; UV λₘₐₓ 215, 277; HRMS m/z 684.4449 [C₃₆H₅₇N₇O₆+H]⁺.

### Example 9

### (S)—N-((3R,4S,5S)-1-((S)-2-((1R,2R)-3-((2-(1H-Imidazol-2-yl)ethyl)amino)-1-methoxy-2-methyl-3-oxopropyl)pyrrolidin-1-yl)-3-methoxy-5-methyl-1-oxoheptan-4-yl)-N,3-dimethyl-2-((S)-3-methyl-2-(methylamino)butanamido)butanamide, TFA salt

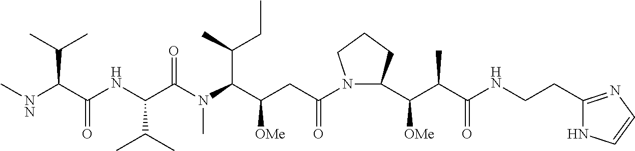

### Step 1. Preparation of Boc-Dap-[2-(H-imidazol-2-yl)ethyl]amine

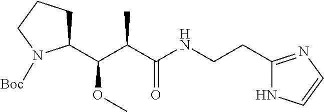 [SMILES: CO[C@H]([C@@H](C)C(=O)NCCc1ncc[nH]1)[C@@H]1CCCN1C(=O)OC(C)(C)C]

To a stirred room temperature suspension of Boc-Dap-OH dicyclohexylamine salt (2.55 g, 5.43 mmol) and [2-(1H-imidazol-2-yl)ethyl]amine dihydrochloride (1.00 g, 5.43 mmol) in CH₂Cl₂ (5 mL) was added DIEA (4.48 mL, 27.2 mmol), followed by DEPC (1.23 mL, 8.15 mmol). After 10 hours, analysis by LCMS showed the reaction was complete. Boc-Dap-[2-(1H-imidazol-2-yl)ethyl]amine was isolated by flash chromatography on silica gel (silica gel 40 μm, 60 Å, 3.0×17.0 cm) using 2% to 10% MeOH/1% NEt₃ in CH₂Cl₂ as the eluent. A total of 1.48 g of Boc-Dap-[2-(1H-imidazol-2-yl)ethyl]amine (3.89 mmol, 72% yield) was obtained.

### Step 2

Fmoc-MeVal-Val-Dil-OtBu (1.20 g, 1.73 mmol) and Boc-Dap-[2-(1H-imidazol-2-yl)ethyl]amine (0.60 g, 1.58 mmol) were suspended in CH₂Cl₂ (5 mL), and to the stirring solution was added TFA (4 mL). After 8 hours, analysis by LCMS showed the reaction was complete. The volatile organics were evaporated in vacuo to yield crude Fmoc-MeVal-Val-Dil-OH and H-Dap-[2-(1H-imidazol-2-yl)ethyl]amine TFA salt, which were used without further purification.

To a stirred room temperature suspension of crude Fmoc-MeVal-Val-Dil-OH and H-Dap-[2-(1H-imidazol-2-yl)ethyl]amine TFA salt in DMF (6 mL) was added DIEA (1.41 mL, 7.90 mmol), followed by HATU (1.80 g, 4.74 mmol) and HOBT (0.48 g, 3.16 mmol). After 12 hours, analysis by LCMS showed the reaction was complete. The reaction was terminated by the addition of a saturated NaHCO₃ solution (10 mL), followed by extraction with EtOAc (4×10 mL). The combined organic extract was washed with brine (20 mL), dried over a pad of magnesium sulfate, filtered, and concentrated in vacuo. The resulting viscous oil was purified by preparatory RP-HPLC, using a Phenomenex Synergi 10V Max-RP 80 Å column (150×30.00 mm) using 10% to 100% acetonitrile in a 0.05% aqueous TFA solution as the eluent. A total of 0.578 g of pure Fmoc-MeVal-Val-Dil-Dap-[2-(1H-imidazol-2-yl)ethyl]amine TFA salt (0.553 mmol, 35% yield) was obtained.

To a stirred room temperature suspension of Fmoc-MeVal-Val-Dil-Dap-[2-(1H-imidazol-2-yl)ethyl]amine TFA salt (0.578 g, 0.553 mmol) in CH₂Cl₂ (5 mL) was added piperidine (2 mL). After 10 hours, analysis by LCMS showed the reaction was complete. The product was purified by preparatory RP-HPLC, using a Phenomenex Synergi 10μ Max-RP 80 Å column (150×30.00 mm) using 10% to 100% acetonitrile in a 0.05% aqueous TFA solution as the eluent. A total of 111 mg of the title compound was obtained as the TFA salt (0.135 mmol, 24% yield). LCMS RT=1.96 minutes; ESI-MS m/z 678.71 [M+H]⁺; UV λₘₐₓ 215. HRMS m/z 678.4918 [C₃₅H₆₃N₇O₆+H]⁺.

### Example 10

### N—((S)-1-(((3R,4S,5S))-1-((S)-2-((1R,2R)-3-((2-(1H-Imidazol-2-yl)ethyl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)-4-aminobenzamide, TFA salt

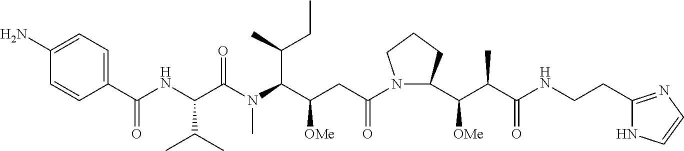 [SMILES: CC[C@H](C)[C@@H]([C@@H](CC(=O)N1CCC[C@H]1[C@H](OC)[C@@H](C)C(=O)NCCc1ncc[nH]1)OC)N(C)C(=O)[C@@H](NC(=O)c1ccc(N)cc1)C(C)C]

4-Abz-Val-Dil-OtBu (0.90 g, 1.89 mmol) and Boc-Dap-2-(1H-imidazole-2-yl)-ethylamine (0.72 g, 1.89 mmol) were suspended in CH₂Cl₂ (5 mL), and to the stirring mixture was added TFA (5 mL). After 8 hours, analysis by LCMS showed the reaction was complete. Volatile organics were evaporated in vacuo to yield crude 4-Abz-Val-Dil-OH TFA salt and H-Dap-2-(1H-imidazole-2-yl)-ethylamine TFA salt, which were used without further purification.

To a stirred room temperature suspension of crude 4-Abz-Val-Dil-OH TFA salt and H-Dap-2-(1H-imidazole-2-yl)-ethylamine TFA salt in DMF (6 mL) was added DIEA (1.35 mL, 7.56 mmol), followed by HATU (1.44 g, 3.78 mmol) and HOBT (0.58 g, 3.78 mmol). After 12 hours, analysis by LCMS showed the reaction was complete. The solvent was removed in vacuo. The resulting viscous oil was purified by preparatory RP-HPLC, using a Phenomenex Synergi 10μ Max-RP 80 Å column (150×30.00 mm) using 10% to 100% acetonitrile in a 0.05% aqueous TFA solution as the eluent. A total of 0.189 g of the title compound was obtained as the TFA salt (0.23 mmol, 12% yield). LCMS RT=2.21 minutes; ESI-MS m/z 684.69 [M+H]⁺; UV λₘₐₓ 215, 270; HRMS m/z 684.4449 [C₃₆H₅₇N₇O₆+H]⁺.

### Example 11

### (S)—N-((3R,4S,5S)-1-((S)-2-((1R,2R)-3-((2-(1H-Imidazol-4-yl)ethyl)amino)-1-methoxy-2-methyl-3-oxopropyl)pyrrolidin-1-yl)-3-methoxy-5-methyl-1-oxoheptan-4-yl)-N,3-dimethyl-2-((S)-3-methyl-2-(methylamino)butanamido)butanamide, TFA salt

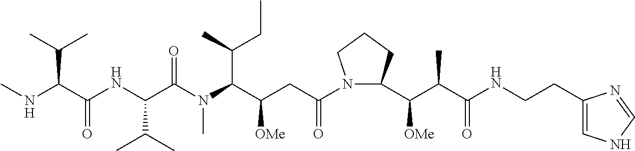 [SMILES: CC[C@H](C)[C@@H]([C@@H](CC(=O)N1CCC[C@H]1[C@H](OC)[C@@H](C)C(=O)NCCc1c[nH]cn1)OC)N(C)C(=O)[C@@H](NC(=O)[C@@H](NC)C(C)C)C(C)C]

### Step 1. Preparation of Boc-Dap-[2-(1H-imidazol-4-yl)ethyl]amine

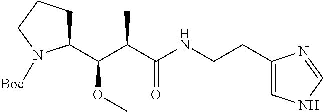 [SMILES: CO[C@H]([C@@H](C)C(=O)NCCc1c[nH]cn1)[C@@H]1CCCN1C(=O)OC(C)(C)C]

To a stirred room temperature suspension of Boc-Dap-OH dicyclohexylamine salt (1.00 g, 2.14 mmol) and [2-(1H-Imidazol-4-yl)ethyl]amine (0.48 g, 4.3 mmol) in CH₂Cl₂ (5 mL) was added DIEA (0.761 mL, 4.27 mmol), followed by DEPC (0.483 mL, 3.20 mmol). After 10 hours, analysis by LCMS showed the reaction was complete. Boc-Dap-[2-(1H-imidazol-4-yl)ethyl]amine was isolated by flash chromatography on silica gel (silica gel 40 μm, 60 Å, 3.0×17.0 cm) using 2% to 10% MeOH/1% NEt₃ in CH₂Cl₂ as the eluent. A total of 0.753 g of Boc-Dap-[2-(1H-Imidazol-4-yl)ethyl]amine (2.0 mmol, 93% yield) was obtained.

### Step 2

Fmoc-MeVal-Val-Dil-OtBu (3.25 g, 4.68 mmol) and Boc-Dap-[2-(1H-imidazol-4-yl)ethyl]amine (1.78 g, 4.68 mmol) were suspended in CH₂Cl₂ (10 mL), and to the stirring mixture was added TFA (5 mL). After 8 hours, analysis by LCMS showed the reaction was complete. The volatile organics were removed in vacuo to yield crude Fmoc-MeVal-Val-Dil-OH and H-Dap-[2-(1H-imidazol-4-yl)ethyl]amine TFA salt which were used without further purification.

To a stirred room temperature Fmoc-MeVal-Val-Dil-OH and H-Dap-[2-(1H-imidazol-4-yl)ethyl]amine TFA salt in DMF (10 mL) was added DIEA (4.17 mL, 23.4 mmol), followed by HATU (3.56 g, 9.36 mmol) and HOBT (1.43 g, 9.36 mmol). After 10 hours, analysis by LCMS showed the reaction was complete. The reaction was terminated by the addition of a saturated NaHCO₃ solution (20 mL), followed by extraction with EtOAc (4×20 mL). The combined organic extract was washed with brine (50 mL), dried over a pad of magnesium sulfate, filtered and concentrated in vacuo. The resulting viscous oil was purified by preparatory RP-HPLC, using a Phenomenex Synergi 10μ Max-RP 80 Å column (150×30.00 mm) using 10% to 100% acetonitrile in a 0.05% aqueous TFA solution as the eluent. A total of 60 mg of pure Fmoc-MeVal-Val-Dil-Dap-[2-(1H-imidazol-4-yl)ethyl]amine TFA salt (0.057 mmol, 1% yield) was obtained.

To a stirred room temperature suspension of Fmoc-MeVal-Val-Dil-Dap-[2-(1H-imidazol-4-yl)ethyl]amine TFA salt (60 mg, 0.057 mmol) in CH₂Cl₂ (2 mL) was added piperidine (1 mL). After 10 hours, analysis by LCMS showed the reaction was complete. The product was purified by preparatory RP-HPLC, using a Phenomenex Synergi 10μ Max-RP 80 Å column (150×30.00 mm) using 10% to 100% acetonitrile in a 0.05% aqueous TFA solution as the eluent. A total of 24 mg of the title compound was obtained as the TFA salt (0.029 mmol, 51% yield). LCMS RT=1.91 minutes; ESI-MS m/z 678.74 [M+H]⁺; 700.59 [M+Na]⁺; UV λₘₐₓ 215, 339; HRMS m/z 678.4918 [C₃₅H₆₃N₇O₆+H]⁺.

### Example 12

### N—((S)-1-(((3R,4S,5S)-1-((S)-2-((1R,2R)-3-((2-(1H-Imidazol-4-yl)ethyl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)-4-aminobenzamide, TFA salt

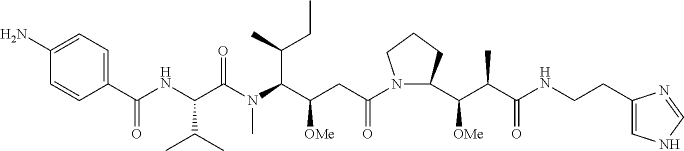 [SMILES: CC[C@H](C)[C@@H]([C@@H](CC(=O)N1CCC[C@H]1[C@H](OC)[C@@H](C)C(=O)NCCc1c[nH]cn1)OC)N(C)C(=O)[C@@H](NC(=O)c1ccc(N)cc1)C(C)C]

Cbz-4-Abz-Val-Dil-OtBu (0.88 g, 1.45 mmol) and Boc-Dap-[2-(1H-imidazol-4-yl)ethyl]amine (0.55 g, 1.45 mmol) were suspended in CH₂Cl₂ (5 mL), and to the stirring mixture was added TFA (5 mL). After 6 hours, analysis by LCMS showed the reaction was complete. The volatile organics were evaporated in vacuo to yield crude Cbz-4-Abz-Val-Dil-OH and H-Dap-[2-(1H-Imidazol-4-yl)ethyl]amine TFA salt that were used without further purification.

To a stirred room temperature suspension of crude Cbz-4-Abz-Val-Dil-OH and H-Dap-[2-(1H-imidazol-4-yl)ethyl]amine TFA salt in DMF (10 mL) was added DIEA (0.78 mL, 4.35 mmol), followed by HATU (1.10 g, 2.90 mmol) and HOBT (0.44 g, 2.90 mmol). After 10 hours, analysis by LCMS showed the reaction was complete. The reaction was terminated by the addition of a saturated NaCHO₃ solution (20 mL), followed by extraction with EtOAc (4×20 mL). The combined organic extract was washed with brine (50 mL), dried over a pad of magnesium sulfate, filtered, and concentrated in vacuo. The resulting viscous oil purified by preparatory RP-HPLC, using a Phenomenex Synergi 10μ Max-RP 80 Å column (150×30.00 mm) using 10% to 100% acetonitrile in a 0.05% aqueous TFA solution as the eluent. A total of 0.327 g of Cbz-4-Abz-Val-Dil-Dap-[2-(1H-imidazol-4-yl)ethyl]amine TFA salt (0.34 mmol, 23% yield) was obtained.

To a stirred room temperature suspension of Cbz-4-Abz-Val-Dil-Dap-[2-(1H-imidazol-4-yl)ethyl]amine (0.327 g, 0.34 mmol) and HCO₂NH₄ (4 mg, 0.60 mmol) in DMF (2 mL) and water (0.3 mL) was added 10% Pd/C (164 mg). The solution was stirred vigorously at room temperature. After 48 hours, analysis by LCMS showed the reaction was complete. The reaction mixture was filtered through a pad of a diatomaceous earth and the filtrate was concentrated to give a total of 238 mg of the crude title compound as the formate salt (0.32 mmol, 94% yield). The product was purified by preparatory RP-HPLC, using a Phenomenex Synergi 10μ Max-RP 80 Å column (150×30.00 mm) using 10% to 100% acetonitrile in a 0.05% aqueous TFA solution as the eluent. A total of 179 mg of the title compound was obtained as the TFA salt (0.22 mmol, 70% yield). LCMS RT=2.13 minutes; ESI-MS m/z 684.69 [M+H]⁺; UV λₘₐₓ 213, 277; HRMS m/z 684.4449 [C₃₆H₅₇N₇O₆+H]⁺.

### Example 13

### (S)—N-((3R,4S,5S)-1-((S)-2-((1R,2R)-3-((2-(Dimethylamino)ethyl)amino)-1-methoxy-2-methyl-3-oxopropyl)pyrrolidin-1-yl)-3-methoxy-5-methyl-1-oxoheptan-4-yl)-N,3-dimethyl-2-((S)-3-methyl-2-(methylamino)butanamido)butanamide, TFA salt

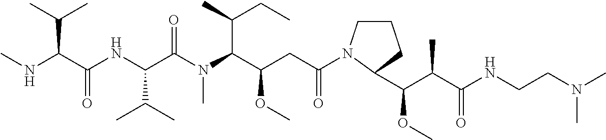

To a stirred room temperature suspension of Boc-Dap-OH dicyclohexylamine salt (3.00 g, 6.41 mmol) and N,N-dimethylethylenediamine (1.40 g, 12.8 mmol) in CH₂Cl₂ (10 mL) was added DIEA (2.28 mL, 12.8 mmol), followed by DEPC (1.45 mL, 9.61 mmol). After 10 hours, analysis by LCMS showed the reaction was complete. The solvent was removed and Boc-Dap-N,N-dimethylethylenediamine was isolated by flash chromatography on silica (silica gel 40 m, 60 Å, 3.0×17.0 cm) using 2% to 10% MeOH in CH₂Cl₂ as the eluent. A total of 1.54 g of Boc-Dap-N,N dimethylethylenediamine (4.31 mmol, 67% yield) was obtained.

To a stirred room temperature suspension of Fmoc-MeVal-Val-Dil-OtBu (4.85 g, 6.99 mmol) and Boc-Dap-N,N-dimethylethylenediamine (2.50 g, 6.99 mmol) in CH₂Cl₂ (10 mL) was added TFA (5 mL). After 8 hours, analysis by LCMS showed the reaction was complete. The volatile organics were evaporated in vacuo to yield crude Fmoc-MeVal-Val-Dil-OH and H-Dap-N,N-dimethylethylenediamine TFA salt that were used without further purification.

To a stirred room temperature suspension of crude H-Dap-N,N-dimethylethylenediamine TFA salt and Fmoc-MeVal-Val-Dil-OH in DMF (10 mL) was added DIEA (4.98 mL, 28.0 mmol), followed by HATU (5.32 g, 14.0 mmol) and HOBT (2.14 g, 14.0 mmol). After 6 hours, analysis by LCMS showed the reaction was complete. The reaction was terminated by the addition of a saturated NaCHO₃ solution (20 mL), followed by extraction with EtOAc (4×20 mL). The combined organic extract was washed with brine (50 mL), dried over a pad of magnesium sulfate, and concentrated in vacuo. The resulting viscous oil was purified by preparatory RP-HPLC, using a Phenomenex Synergi 10V Max-RP 80 Å column (150×30.00 mm) using 10% to 100% acetonitrile in a 0.05% aqueous TFA solution as the eluent. A total of 3.75 g of Fmoc-MeVal-Val-Dil-Dap-N,N-dimethylethylenediamine TFA salt (3.67 mmol, 52% yield) was obtained.

To a stirred room temperature suspension of Fmoc-MeVal-Val-Dil-Dap-N,N-dimethylethylenediamine TFA salt (1.97 g, 1.93 mmol) in CH₂Cl₂ (5 mL) was added piperidine (2 mL). After 10 hours, analysis by LCMS showed the reaction was complete. The product was purified by preparatory RP-HPLC, using a Phenomenex Synergi 10μ Max-RP 80 Å column (150×30.00 mm) using 10% to 100% acetonitrile in a 0.05% aqueous TFA solution as the eluent. A total of 0.963 g of the title compound was obtained as the TFA salt (1.21 mmol, 62% yield). LCMS RT=1.83 minutes; ESI-MS m/z 655.76 [M+H]⁺; UV λₘₐₓ 212; HRMS m/z 655.5122 [C₃₄H₆₆N₆O₆+H]⁺.

### Example 14

### (S)-2-((S)-2-(Dimethylamino)-3-methylbutanamido)-N-((3R,4S,5S)-3-methoxy-1-((S)-2-((1R,2R)-1-methoxy-2-methyl-3-((2-(methylamino)ethyl)amino)-3-oxopropyl)pyrrolidin-1-yl)-5-methyl-1-oxoheptan-4-yl)-N,3-dimethylbutanamide, TFA salt

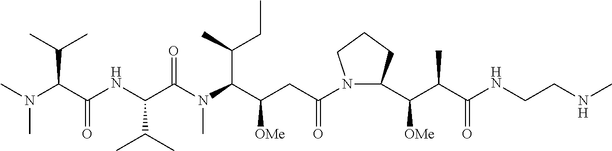

### Step 1. Preparation of Dov-Dil-OtBu

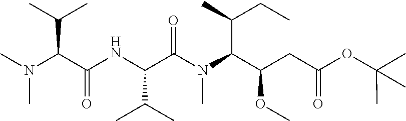 [SMILES: CC[C@H](C)[C@@H]([C@@H](CC(=O)OC(C)(C)C)OC)N(C)C(=O)[C@@H](NC(=O)[C@H](C(C)C)N(C)C)C(C)C]

To stirred a room temperature solution of crude Fmoc-Val-Dil-OtBu (13.3 g, 22.8 mmol) in CH₂Cl₂ (20 mL) was added piperidine (15 mL). After 8 hours, analysis by LCMS showed the reaction was complete. Volatile organics were evaporated in vacuo to yield crude H-Val-Dil-OtBu that was used without further purification.

To a stirred room temperature suspension of crude H-Val-Dil-OtBu and Dov (6.63 g, 45.7 mmol) in CH₂Cl₂ (20 mL) was added DIEA (12.2 mL, 68.5 mmol), followed by DEPC (10.3 mL, 68.5 mmol). After 12 hours, analysis by LCMS showed the reaction was complete. The solvent was evaporated in vacuo, the crude mixture dissolved in EtOAc (50 mL), and washed with water (250 mL×2), and brine (250 mL). The organic fraction was filtered through a pad of magnesium sulfate and concentrated in vacuo. The resulting viscous oil was purified by preparatory RP-HPLC with a Phenomenex Synergi 10μ Max-RP 80 Å column (150×30.00 mm) using a 10% to 100% acetonitrile in 0.05% aqueous TFA eluent. A total of 9.58 g of Dov-Val-Dil-OtBu TFA salt (15.2 mmol, 67% yield) was obtained.

### Step 2

To a stirred room temperature solution of N′-Boc-N-methylaminoethylamine (1.00 g, 4.75 mmol) stirring in CH₂Cl₂ (10 mL) was added Fmoc-OSu (1.76 g, 5.22 mmol) and DIEA (1.69 g, 9.49 mmol). After 1 hour, analysis by LCMS showed the reaction was complete. The solvent was removed in vacuo and the viscous reaction mixture dissolved in EtOAc (100 mL) and washed with a 0.1 M HCl solution (50 mL), a saturated NaHCO₃ solution (50 mL), and brine (50 mL). The organic fraction was dried over a pad of magnesium sulfate, filtered, and concentrated in vacuo. The crude N′-Boc-N-Fmoc-N-methylaminoethylamine was used without further purification.

To stirred a room temperature solution of crude N′-Boc-N-Fmoc-N-methylaminoethylamine in CH₂Cl₂ (5 mL) was added TFA (2 mL). After 10 hours, analysis by LCMS showed the reaction was complete. The solvent was removed in vacuo and the crude product was purified by preparatory RP-HPLC, using a Phenomenex Synergi 10μ Max-RP 80 Å column (150×30.00 mm) using 10% to 100% acetonitrile in a 0.05% aqueous TFA solution as the eluent. A total of 2.04 g of N-Fmoc-N-Methylaminoethylamine TFA salt (3.89 mmol, 79% yield) was obtained.

To a stirred room temperature suspension of Boc-Dap-OH dicyclohexylamine salt (0.88 g, 1.89 mmol) and N-Fmoc-N-methylaminoethylamine TFA salt (1.04 g, 2.83 mmol) in CH₂Cl₂ (10 mL) was added DIEA (1.01 mL, 5.66 mmol), followed by DEPC (0.43 mL, 2.83 mmol). After 10 hours, analysis by LCMS showed the reaction was complete. The solvent was removed and the Boc-Dap-N-Fmoc-N-methylaminoethylamine was isolated by flash chromatography on silica gel (silica gel 40 μm, 60 Å, 3.0×17.0 cm) using 2% to 10% MeOH as the eluent. A total of 0.617 g of Boc-Dap-N-Fmoc-N-methylaminoethylamine (1.09 mmol, 58% yield) was obtained.

### Step 3

To a stirred room temperature suspension of Dov-Val-Dil-OtBu (0.636 g, 1.31 mmol) and Boc-Dap-N-Fmoc-N-methylaminoethylamine (0.617 g, 1.09 mmol) in CH₂Cl₂ (5 mL) was added TFA (4 mL). After 8 hours, analysis by LCMS showed the reaction was complete. The volatile organics were removed in vacuo, to yield crude Dov-Val-Dil-OH and H-Dap-N-Fmoc-N-methylaminoethylamine TFA salt that were used without further purification.

To a stirred room temperature suspension of crude H-Dap-N-Fmoc-N-methylaminoethylamine TFA salt and Dov-Val-Dil-OH TFA salt in DMF (6 mL) was added DIEA (0.97 mL, 5.45 mmol), followed by HATU (0.83 g, 2.18 mmol) and HOBT (0.33 g, 2.18 mmol). After 6 hours, analysis by LCMS showed the reaction was complete. The reaction was terminated by the addition of a saturated NaHCO₃ solution (10 mL), followed by extraction with EtOAc (4×10 mL). The combined organic extract was washed with brine (20 mL), dried over a pad magnesium sulfate, filtered, and concentrated in vacuo. The resulting viscous oil purified by preparatory RP-HPLC, using a Phenomenex Synergi 10V Max-RP 80 Å column (150×30.00 mm) using 10% to 100% acetonitrile in a 0.05% aqueous TFA solution as the eluent. A total of 0.437 g of Dov-Val-Dil-Dap-N-Fmoc-N-methylaminoethylamine TFA salt (0.428 mmol, 39% yield) was obtained.

To a stirred room temperature suspension of Dov-Val-Dil-Dap-N-Fmoc-N-methylaminoethylamine TFA salt (0.437 g, 0.428 mmol) in CH₂Cl₂ (3 mL) was added piperidine (1 mL). After 10 hours, analysis by LCMS showed the reaction was complete. The product was purified by preparatory RP-HPLC, using a Phenomenex Synergi 10μ Max-RP 80 Å column (150×30.00 mm) using 10% to 100% acetonitrile in a 0.05% aqueous TFA solution as the eluent. A total of 0.212 g of the title compound was obtained as the TFA salt (0.265 mmol, 62% yield). LCMS RT=1.98 minutes; ESI-MS m/z 655.80 [M+H]⁺; HRMS m/z 655.5122 [C₃₄H₆₆N₆O₆+H]⁺.

### Example 15

### (S)—N-((3R,4S,5S)-1-((S)-2-((1R,2R)-3-((2-Aminoethyl)amino)-1-methoxy-2-methyl-3-oxopropyl)pyrrolidin-1-yl)-3-methoxy-5-methyl-1-oxoheptan-4-yl)-2-((S)-2-(dimethylamino)-3-methylbutanamido)-N,3-dimethylbutanamide, TFA salt

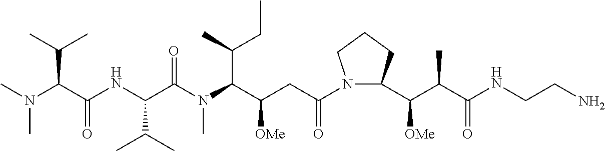 [SMILES: CC[C@H](C)[C@@H]([C@@H](CC(=O)N1CCC[C@H]1[C@H](OC)[C@@H](C)C(=O)NCCN)OC)N(C)C(=O)[C@@H](NC(=O)[C@H](C(C)C)N(C)C)C(C)C]

To a stirred room temperature suspension of Boc-Dap-OH dicyclohexylamine salt (2.04 g, 6.40 mmol) and Fmoc-ethylenediamine hydrochloride (3.00 g, 6.40 mmol) in CH₂Cl₂ (10 mL) was added DIEA (4.56 mL, 25.6 mmol), followed by DEPC (1.45 mL, 9.60 mmol). After 10 hours, analysis by LCMS showed the reaction was complete. The solvent was removed in vacuo, and the Boc-Dap-ethylenediamine-Fmoc was isolated by flash chromatography on silica gel (silica gel 40 μm, 60 Å, 3.0×17.0 cm) using 2% to 10% MeOH in CH₂Cl₂ as the eluent. A total of 1.38 g of Boc-Dap-ethylenediamine-Fmoc (2.50 mmol, 39% yield) was obtained.

To a stirred room temperature suspension of Boc-Dap-ethylenediamine-Fmoc (1.38 g, 2.50 mmol) and Dov-Val-Dil-OtBu (1.65 g, 2.75 mmol) in CH₂Cl₂ (5 mL) was added TFA (4 mL). After 8 hours, analysis by LCMS showed the reaction was complete. The volatile organics were evaporated in vacuo to yield crude Dov-Val-Dil-OH TFA salt and H-Dap-ethylenediamine-Fmoc TFA salt that were used without further purification.

To a stirred room temperature suspension of crude H-Dap-ethylenediamine-Fmoc TFA salt and Dov-Val-Dil-OH TFA salt in EtOAc (10 mL) was added DIEA (1.78 mL, 10.0 mmol), followed by DEPC (1.51 g, 10.0 mmol). After 18 hours, analysis by LCMS showed the reaction was complete. The crude mixture was washed with a saturated NaHCO₃ solution (50 mL), and water (50 mL×2). The organic fraction was filtered through a pad of magnesium sulfate and concentrated in vacuo. The resulting viscous oil was purified by flash chromatography on silica gel (silica gel 40 μm, 60 Å, 3.0×17.0 cm) using 5% to 10% MeOH in CH₂Cl₂ as the eluent. A total of 1.04 g of Dov-Val-Dil-Dap-ethylenediamine-Fmoc (1.20 mmol, 48% yield) was obtained.

To a stirred room temperature suspension of Dov-Val-Dil-Dap-ethylenediamine-Fmoc (1.04 g, 1.20 mmol) in CH₂Cl₂ (5 mL) was added piperidine (2 mL). After 10 hours, analysis by LCMS showed the reaction was complete. The solvent was removed and the product was purified by preparatory RP-HPLC, using a Phenomenex Synergi 10μ Max-RP 80 Å column (150×30.00 mm) using 10% to 100% acetonitrile in a 0.05% aqueous TFA solution as the eluent. A total of 0.716 g of the title compound was obtained as the TFA salt (0.912 mmol, 76% yield). LCMS RT=1.89 minutes; ESI-MS m/z 641.64 [M+H]⁺; HRMS m/z 641.4966 [C₃₃H₆₄N₆O₆+H]⁺.

### Example 16

### (S)—N-((3R,4S,5S)-3-Methoxy-1-((S)-2-((1R,2R)-1-methoxy-2-methyl-3-((2-morpholinoethyl)amino)-3-oxopropyl)pyrrolidin-1-yl)-5-methyl-1-oxoheptan-4-yl)-N,3-dimethyl-2-((S)-3-methyl-2-(methylamino)butanamido)butanamide, TFA salt

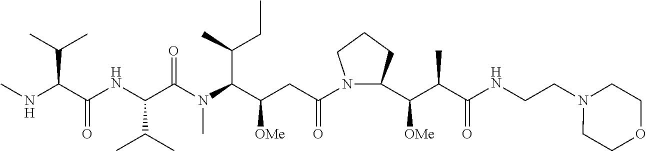 [SMILES: CC[C@H](C)[C@@H]([C@@H](CC(=O)N1CCC[C@H]1[C@H](OC)[C@@H](C)C(=O)NCCN1CCOCC1)OC)N(C)C(=O)[C@@H](NC(=O)[C@@H](NC)C(C)C)C(C)C]

### Step 1. Preparation of Boc-Dap-4-(2-aminoethyl)morpholine

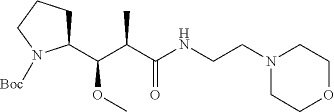 [SMILES: CO[C@H]([C@@H](C)C(=O)NCCN1CCOCC1)[C@@H]1CCCN1C(=O)OC(C)(C)C]

To a stirred room temperature suspension of Boc-Dap-OH dicyclohexylamine salt (3.00 g, 6.41 mmol) and 4-(2-aminoethyl)morpholine (0.92 g, 7.1 mmol) in CH₂Cl₂ (10 mL) was added DIEA (2.28 mL, 12.8 mmol), followed by DEPC (1.45 mL, 9.61 mmol). After 10 hours, analysis by LCMS showed the reaction was complete. Boc-Dap-4-(2-aminoethyl)morpholine was isolated by flash chromatography on silica gel (silica gel 40 μm, 60 Å, 3.0×17.0 cm) using 2% to 10% MeOH in CH₂Cl₂ as the eluent. A total of 2.41 g of Boc-Dap-4-(2-aminoethyl)morpholine (6.03 mmol, 94% yield) was obtained.

### Step 2

To a stirred room temperature suspension of Fmoc-MeVal-Val-Dil-OtBu (5.42 g, 7.81 mmol) and Boc-Dap-4-(2-aminoethyl)morpholine (3.12 g, 7.81 mmol) in CH₂Cl₂ (10 mL) was added TFA (10 mL). After 8 hours, analysis by LCMS showed the reaction was complete. The volatile organics were evaporated in vacuo to yield crude Fmoc-MeVal-Val-Dil-OH and H-Dap-4-(2-aminoethyl)morpholine TFA salt that were used without further purification.

To a stirred room temperature suspension of crude Fmoc-MeVal-Val-Dil-OH and H-Dap-4-(2-aminoethyl)morpholine TFA salt in DMF (10 mL) was added DIEA (6.96 mL, 39.1 mmol), followed by HATU (5.93 g, 15.6 mmol) and HOBT (2.39 g, 15.6 mmol). After 8 hours, analysis by LCMS showed the reaction was complete. The reaction was terminated by addition of saturated aqueous NaHCO₃ (20 mL), followed by extraction with EtOAc (4×20 mL). The combined organic extract was washed with brine (50 mL), dried over a pad of magnesium sulfate, filtered, and concentrated in vacuo. The resulting viscous oil was purified by preparatory RP-HPLC, using a Phenomenex Synergi 10μ Max-RP 80 Å column (150×30.00 mm) using 10% to 100% acetonitrile in a 0.05% aqueous TFA solution as the eluent. A total of 0.578 g of Fmoc-MeVal-Val-Dil-Dap-4-(2-aminoethyl)morpholine TFA salt (0.544 mmol, 7% yield) was obtained.

To a stirred room temperature suspension of Fmoc-MeVal-Val-Dil-Dap-4-(2-aminoethyl)morpholine TFA salt 0.578 g, 0.544 mmol) in CH₂Cl₂ (4 mL) was added piperidine (2 mL). After 10 hours, analysis by LCMS showed the reaction was complete. The solvent was removed in vacuo and the product was purified by preparatory RP-HPLC, using a Phenomenex Synergi 10μ Max-RP 80 Å column (150×30.00 mm) using 10% to 100% acetonitrile in a 0.05% aqueous TFA solution as the eluent. A total of 0.246 g of the title compound was obtained as the TFA salt (0.293 mmol, 54% yield). LCMS RT=1.87 minutes; ESI-MS m/z 697.73 [M+H]⁺; HRMS m/z 697.5228 [C₃₆H₆₈N₆O₇+H]⁺.

### Example 17

### (S)-2-((S)-2-(Dimethylamino)-3-methylbutanamido)-N-((3R,4S,5S)-3-methoxy-1-((S)-2-((1R,2R)-1-methoxy-2-methyl-3-oxo-3-((2-(piperazin-1-yl)ethyl)amino)propyl)pyrrolidin-1-yl)-5-methyl-1-oxoheptan-4-yl)-N,3-dimethylbutanamide, TFA salt

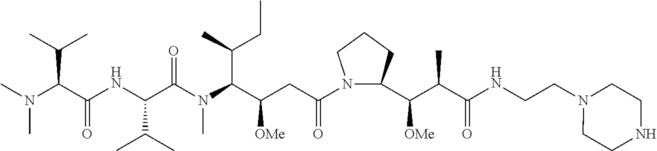 [SMILES: CC[C@H](C)[C@@H]([C@@H](CC(=O)N1CCC[C@H]1[C@H](OC)[C@@H](C)C(=O)NCCN1CCNCC1)OC)N(C)C(=O)[C@@H](NC(=O)[C@H](C(C)C)N(C)C)C(C)C]

To a stirred room temperature solution of 1-(2-aminoethyl)piperazine (4.00 g, 31.0 mmol) in CH₂Cl₂ (170 mL) was added Cbz-OSu (7.73 g, 31.0 g) followed by DIEA (5.40 mL, 31.0 mmol) and N,N-dimethylaminopyridine (DMAP; 10 mg). After 24 hours, analysis by LCMS showed the reaction was complete. The slightly yellow reaction mixture was washed sequentially with a 0.1 M HCl solution (150 mL), a saturated aqueous NaHCO₃ solution (100 mL), and brine (100 mL). The organic fraction was dried over a pad of magnesium sulfate, filtered, and concentrated in vacuo. The crude product was isolated by flash chromatography on silica gel (silica gel 40 m, 60 Å, 9.0×20.0 cm) using 2% to 10% MeOH in CH₂Cl₂ as the eluent. The isolated product was further purified by preparatory RP-HPLC, using a Phenomenex Synergi 10V Max-RP 80 Å column (150×30.00 mm) using 10% to 100% acetonitrile in a 0.05% aqueous TFA solution as the eluent. A total of 0.862 g of 2-(4-Cbz-piperazin-1-yl)ethanamine TFA salt (2.28 mmol, 7% yield) was obtained.

To a stirred room temperature suspension of Boc-Dap-OH dicyclohexylamine salt (1.07 g, 2.28 mmol) and 2-(4-Cbz-piperazin-1-yl)ethanamine TFA salt (0.86 g, 2.28 mmol) in CH₂Cl₂ (6 mL) was added DIEA (1.22 mL, 6.85 mmol), followed by DEPC (0.86 mL, 5.71 mmol). After 10 hours, analysis by LCMS showed the reaction was complete. The solvent was removed in vacuo and the Boc-Dap-2-(4-Cbz-piperazin-1-yl)ethanamine was isolated by flash chromatography on silica gel (silica gel 40 m, 60 Å, 3.0×17.0 cm) using 2% to 10% MeOH in CH₂Cl₂ as the eluent. A total of 0.96 g of Boc-Dap-2-(4-Cbz-piperazin-1-yl)ethanamine (1.80 mmol, 79% yield) was obtained.

To a stirred room temperature suspension of Boc-Dap-2-(4-Cbz-piperazin-1-yl)ethanamine (0.31 g, 0.59 mmol) and Dov-Val-Dil-OtBu (0.314 g, 0.65 mmol) in CH₂Cl₂ (5 mL) was added TFA (4 mL). After 8 hours, analysis by LCMS showed the reaction was complete. The volatile organics were evaporated in vacuo to yield crude Dov-Val-Dil-OH TFA salt and H-Dap-2-(4-Cbz-piperazin-1-yl)ethanamine TFA salt that were used without further purification.

To a stirred room temperature suspension of crude Dov-Val-Dil-OH TFA salt and H-Dap-2-(4-Cbz-piperazin-1-yl)ethanamine TFA salt in DMF (5 mL) was added DIEA (0.42 mL, 2.35 mmol), followed by HATU (0.45 g, 1.18 mmol) and HOBT (0.18 g, 1.18 mmol). After 10 hours, analysis by LCMS showed the reaction was complete. The reaction was terminated by the addition of a saturated NaHCO₃ solution (20 mL), followed by extraction with EtOAc (4×20 mL). The combined organic extract was washed with brine (50 mL), dried over a pad of magnesium sulfate, filtered, and concentrated in vacuo. The resulting viscous oil was purified by preparatory RP-HPLC, using a Phenomenex Synergi 10V Max-RP 80 Å column (150×30.00 mm) using 10% to 100% acetonitrile in a 0.05% aqueous TFA solution as the eluent. A total of 29 mg of Dov-Val-Dil-Dap-2-(4-Cbz-piperazin-1-yl)ethanamine TFA salt (0.029 mmol, 5% yield) was obtained.

To a stirred room temperature suspension of Dov-Val-Dil-Dap-2-(4-Cbz-piperazin-1-yl)ethanamine TFA salt (29 mg, 0.029 mmol) and NH₄HCO₂ (3 mg, 0.051 mmol) in DMF (3 mL) and water (0.5 mL) was added 10% Pd/C (17 mg). The solution was stirred vigorously at room temperature. After 48 hours, analysis by LCMS showed the reaction was complete. The reaction mixture was filtered through a pad of a diatomaceous earth and the filtrate was concentrated to give a total of 18 mg of the of the crude title compound as the formate salt (0.023 mmol, 79% yield). The product was purified by preparatory RP-HPLC, using a Phenomenex Synergi 10μ Max-RP 80 Å column (150×30.00 mm) using 10% to 100% acetonitrile in a 0.05% aqueous TFA solution as the eluent. A total of 15 mg of the title compound was obtained as the TFA salt (0.018 mmol, 79% yield). LCMS RT=1.87 minutes; ESI-MS m/z 710.81 [M+H]⁺; HRMS m/z 710.5544 [C₃₇H₇₁N₇O₆+H]⁺.

### Example 18

### (S)—N-((3R,4S,5S)-3-Methoxy-1-((S)-2-((1R,2R)-1-methoxy-2-methyl-3-oxo-3-((2-(piperazin-1-yl)ethyl)amino)propyl)pyrrolidin-1-yl)-5-methyl-1-oxoheptan-4-yl)-N,3-dimethyl-2-((S)-3-methyl-2-(methylamino)butanamido)butanamide

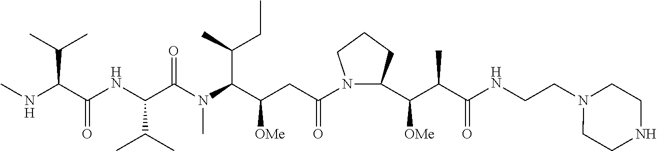 [SMILES: CC[C@H](C)[C@@H]([C@@H](CC(=O)N1CCC[C@H]1[C@H](OC)[C@@H](C)C(=O)NCCN1CCNCC1)OC)N(C)C(=O)[C@@H](NC(=O)[C@@H](NC)C(C)C)C(C)C]

The title compound may be prepared using methods analogous to those described in the Examples and general synthetic schemes.

### Example 19

### (S)—N-((3R,4S,5S)-3-Methoxy-1-((S)-2-((1R,2R)-1-methoxy-2-methyl-3-((2-(4-methylpiperazin-1-yl)ethyl)amino)-3-oxopropyl)pyrrolidin-1-yl)-5-methyl-1-oxoheptan-4-yl)-N,3-dimethyl-2-((S)-3-methyl-2-(methylamino)butanamido)butanamide, TFA salt

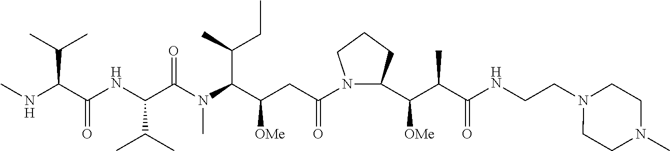

To a stirred room temperature suspension of Boc-Dap-OH dicyclohexylamine salt (3.27 g, 6.99 mmol) and 2-(4-methyl-piperazin-1-yl)-ethylamine (1.00 g, 6.99 mmol) in CH₂Cl₂ (10 mL) was added DIEA (2.49 mL, 14.0 mmol), followed by DEPC (1.58 mL, 10.5 mmol). After 10 hours, analysis by LCMS showed the reaction was complete. The solvent was removed in vacuo, and Boc-Dap-2-(4-methyl-piperazin-1-yl)-ethylamine was isolated by flash chromatography on silica gel (silica gel 40 μm, 60 Å, 3.0×17.0 cm) using 2% to 10% MeOH in CH₂Cl₂ as the eluent. A total of 1.69 g of Boc-Dap-2-(4-methyl-piperazin-1-yl)-ethylamine (4.10 mmol, 59% yield) was obtained.

To a stirred room temperature suspension of Fmoc-MeVal-Val-Dil-OtBu (3.55 g, 5.11 mmol) and Boc-Dap-2-(4-methyl-piperazin-1-yl)-ethylamine (2.11 g, 5.11 mmol) in CH₂Cl₂ (10 mL) was added TFA (10 mL). After 8 hours, analysis by LCMS showed the reaction was complete. The volatile organics were evaporated in vacuo to yield crude Fmoc-MeVal-Val-Dil-OH and H-Dap-2-(4-methyl-piperazin-1-yl)-ethylamine TFA salt that were used without further purification.

To a stirred room temperature suspension of crude Fmoc-MeVal-Val-Dil-OH and H-Dap-2-(4-methyl-piperazin-1-yl)-ethylamine TFA salt in DMF (10 mL) was added DIEA (4.55 mL, 25.6 mmol), followed by HATU (5.93 g, 10.2 mmol) and HOBT (2.39 g, 10.2 mmol). After 12 hours, analysis by LCMS showed the reaction was complete. Reaction was terminated by the addition of a saturated aqueous NaHCO₃ solution (20 mL), followed by extraction with EtOAc (4×20 mL). The combined organic extract was washed with brine (50 mL), dried over a pad of magnesium sulfate, filtered, and concentrated in vacuo. The resulting viscous oil was purified by preparatory RP-HPLC, using a Phenomenex Synergi 10μ Max-RP 80 Å column (150×30.00 mm) using 10% to 100% acetonitrile in a 0.05% aqueous TFA solution as the eluent. A total of 0.533 g of Fmoc-MeVal-Val-Dil-Dap-2-(4-methyl-piperazin-1-yl)-ethylamine TFA salt (0.495 mmol, 10% yield) was obtained.

To a stirred room temperature suspension of Fmoc-MeVal-Val-Dil-Dap-2-(4-methyl-piperazin-1-yl)-ethylamine TFA salt (0.533 g, 0.495 mmol) in CH₂Cl₂ (4 mL) was added piperidine (2 mL). After 10 hours, analysis by LCMS showed the reaction was complete. The solvent was removed in vacuo and the product was purified by preparatory RP-HPLC, using a Phenomenex Synergi 10μ Max-RP 80 Å column (150×30.00 mm) using 10% to 100% acetonitrile in a 0.05% aqueous TFA solution as the eluent. A total of 0.268 g of the title compound was obtained as the TFA salt (0.314 mmol, 63% yield). LCMS RT=1.81 minutes; ESI-MS m/z 710.78 [M+H]⁺; HRMS m/z 710.5544 [C₃₇H₇₁N₇O₆+H]⁺.

### Example 20

### 4-Amino-N—((S)-1-(((3R,4S,5S)-3-methoxy-1-((S)-2-((1R,2R)-1-methoxy-2-methyl-3-((2-morpholinoethyl)amino)-3-oxopropyl)pyrrolidin-1-yl)-5-methyl-1-oxoheptan-4-yl)(methyl)amino)-3-methyl-1-oxobutan-2-yl)benzamide, TFA salt

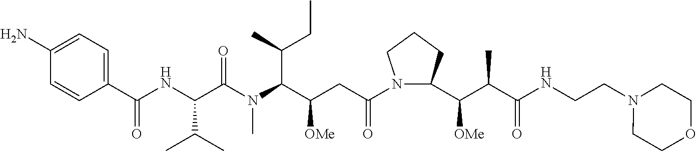 [SMILES: CC[C@H](C)[C@@H]([C@@H](CC(=O)N1CCC[C@H]1[C@H](OC)[C@@H](C)C(=O)NCCN1CCOCC1)OC)N(C)C(=O)[C@@H](NC(=O)c1ccc(N)cc1)C(C)C]

To a stirred room temperature suspension of 4-Abz-Val-Dil-OtBu (1.40 g, 2.02 mmol) and Boc-Dap-4-(2-aminoethyl)morpholine (0.81 g, 2.02 mmol) in CH₂Cl₂ (5 mL) was added TFA (5 mL). After 8 hours, analysis by LCMS showed the reaction was complete. The volatile organics were evaporated in vacuo to yield crude 4-Abz-Val-Dil-OH TFA salt and H-Dap-4-(2-aminoethyl)morpholine TFA salt that were used without further purification.

To a stirred room temperature suspension of crude 4-Abz-Val-Dil-OH TFA salt and H-Dap-4-(2-aminoethyl)morpholine TFA salt in DMF (10 mL) was added DIEA (1.44 mL, 8.08 mmol), followed by HATU (2.30 g, 6.06 mmol) and HOBT (0.62 g, 4.04 mmol). After 12 hours, analysis by LCMS showed the reaction was complete. The solvents was evaporated in vacuo and the resulting viscous oil was purified by preparatory RP-HPLC, using a Phenomenex Synergi Max-RP 80 Å column (150×30.00 mm) using 10% to 100% acetonitrile in a 0.05% aqueous TFA solution as the eluent. A total of 52 mg of the title compound was obtained as the TFA salt (0.061 mmol, 3% yield) was obtained. LCMS RT=2.17 minutes; ESI-MS m/z 703.71 [M+H]⁺; UV λₘₐₓ 215, 277; HRMS m/z 703.4758 [C₃₇H₆₂N₆O₇+H]⁺.

### Example 21

### 4-Amino-N—((S)-1-(((3R,4S,5S)-3-methoxy-1-((S)-2-((1R,2R)-1-methoxy-2-methyl-3-oxo-3-((2-(piperazin-1-yl)ethyl)amino)propyl)pyrrolidin-1-yl)-5-methyl-1-oxoheptan-4-yl)(methyl)amino)-3-methyl-1-oxobutan-2-yl)benzamide

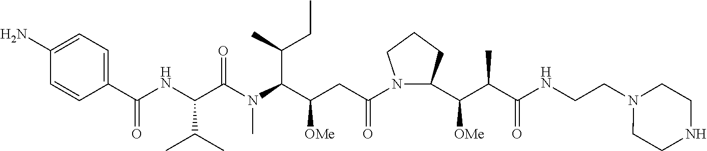 [SMILES: CC[C@H](C)[C@@H]([C@@H](CC(=O)N1CCC[C@H]1[C@H](OC)[C@@H](C)C(=O)NCCN1CCNCC1)OC)N(C)C(=O)[C@@H](NC(=O)c1ccc(N)cc1)C(C)C]

The title compound may be prepared using methods analogous to those described in the Examples and general synthetic schemes.

### Example 22

### (S)—N-((3R,4S,5S)-1-((S)-2-((1R,2R)-3-((4-aminophenethyl)amino)-1-methoxy-2-methyl-3-oxopropyl)pyrrolidin-1-yl)-3-methoxy-5-methyl-1-oxoheptan-4-yl)-2-((S)-2-(dimethylamino)-3-methylbutanamido)-N,3-dimethylbutanamide, TFA salt

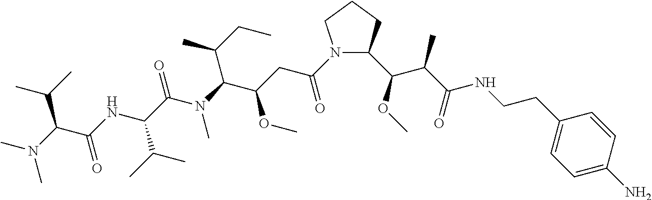 [SMILES: CC[C@H](C)[C@@H]([C@@H](CC(=O)N1CCC[C@H]1[C@H](OC)[C@@H](C)C(=O)NCCc1ccc(N)cc1)OC)N(C)C(=O)[C@@H](NC(=O)[C@H](C(C)C)N(C)C)C(C)C]

To a stirred room temperature solution of 4-(2-aminoethyl)aniline (2.61 mL, 19.8 mmol) in 10% acetic acid (120 mL) was added Fmoc-C1 (5.40 g, 20.9 mmol) in 1,4-dioxane (120 mL). After 24 hours, analysis by LCMS showed the reaction was complete. Non-polar impurities were removed by extraction with diethyl ether (100 mL×2). The aqueous reaction mixture was acidified using a 2 N HCl solution. The precipitate formed was collected, washed with diethyl ether (100 mL) and dried to yield Fmoc-4-(2-aminoethyl)aniline HCl salt (6.57 g, 16.6 mmol, 84% yield).

To a stirred room temperature suspension of Boc-Dap-OH dicyclohexylamine salt (1.00 g, 2.14 mmol) and Fmoc-4-(2-aminoethyl)aniline HCl salt (1.53 g, 3.86 mmol) in CH₂Cl₂ (5 mL) was added DIEA (0.76 mL, 4.27 mmol), followed by DEPC (0.48 mL, 3.20 mmol). After 10 hours, analysis by LCMS showed the reaction was complete. The solvent was removed in vacuo and Boc-Dap-4-(2-aminoethyl)aniline-Fmoc was isolated by flash chromatography on silica gel (silica gel 40 m, 60 Å, 3.0×17.0 cm) using 2% to 10% MeOH in CH₂Cl₂ as the eluent. A total of 1.25 g of Boc-Dap-4-(2-aminoethyl)aniline-Fmoc (1.99 mmol, 93% yield) was obtained.

To a stirred room temperature suspension of Boc-Dap-4-(2-aminoethyl)aniline-Fmoc (1.25 g, 1.99 mmol) and Dov-Val-Dil-OtBu (1.19 g, 1.99 mmol) in CH₂Cl₂ (5 mL) was added TFA (2 mL). After 8 hours, analysis by LCMS showed the reaction was complete. The volatile organics were evaporated in vacuo to yield crude Dov-Val-Dil-OH TFA salt and H-Dap-Fmoc-4-(2-aminoethyl)aniline TFA salt that were used without further purification.

To a stirred room temperature suspension of crude Dov-Val-Dil-OH TFA salt and H-Dap-4-(2-aminoethyl)aniline-Fmoc TFA salt in EtOAc (10 mL) was added DIEA (1.42 mL, 7.96 mmol), followed by DEPC (1.20 g, 7.96 mmol). After 18 hours, analysis by LCMS showed the reaction was complete. The reaction mixture was washed with a saturated NaHCO₃ solution (50 mL), and water (50 mL×2). The organic fraction was dried over a pad of magnesium sulfate, filtered, and concentrated in vacuo. The resulting viscous oil was purified by flash chromatography on silica gel (silica gel 40 μm, 60 Å, 3.0×17.0 cm) using 5% to 10% MeOH in CH₂Cl₂ as the eluent. A total of 1.75 g of Dov-Val-Dil-Dap-4-(2-aminoethyl)aniline-Fmoc (1.86 mmol, 94% yield) was obtained.

To a stirred room temperature suspension of Dov-Val-Dil-Dap-4-(2-aminoethyl)aniline-Fmoc (1.75 g, 1.86 mmol) in CH₂Cl₂ (5 mL) was added piperidine (2 mL). After 10 hours, analysis by LCMS showed the reaction was complete. The solvent was removed in vacuo and the product was purified by preparatory RP-HPLC, using a Phenomenex Synergi 10μ Max-RP 80 Å column (150×30.00 mm) using 10% to 100% acetonitrile in a 0.05% aqueous TFA solution as the eluent. A total of 0.759 g of the title compound as the TFA salt (0.881 mmol, 47% yield). LCMS RT=1.86 minutes; ES-MS m/z 717.66 [M+H]⁺; 740.70 [M+Na]⁺; HRMS m/z 717.5278 [C₃₉H₆₈N₆O₆+H]⁺.

### Example 23

### In vitro Cytotoxicity Experiments

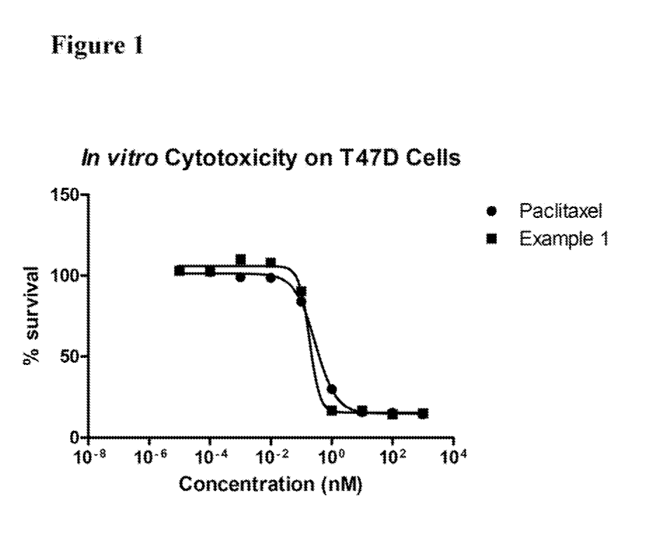

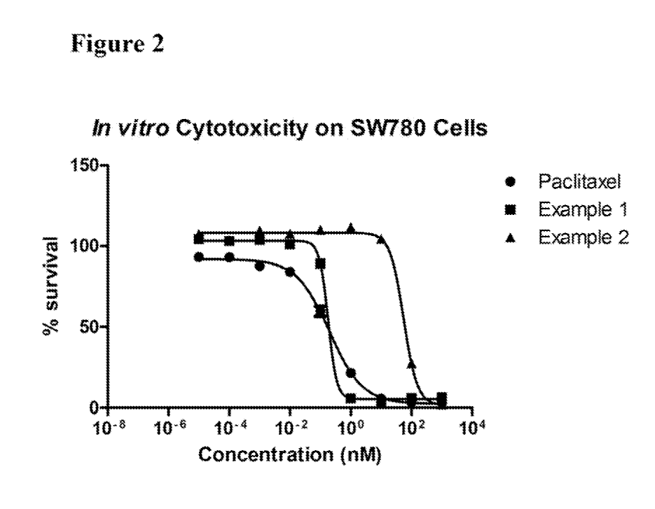

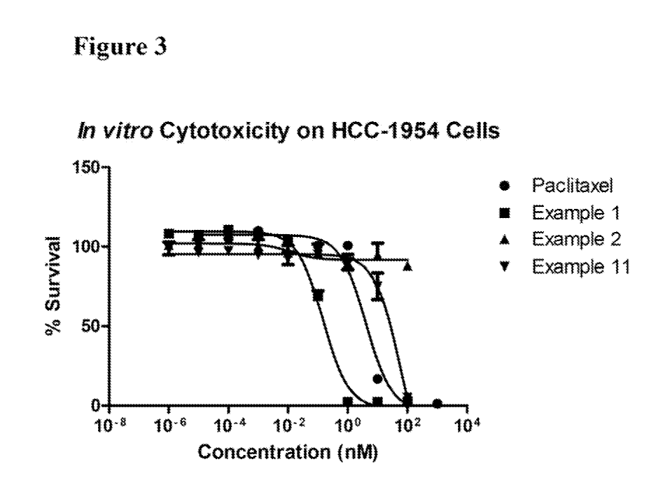

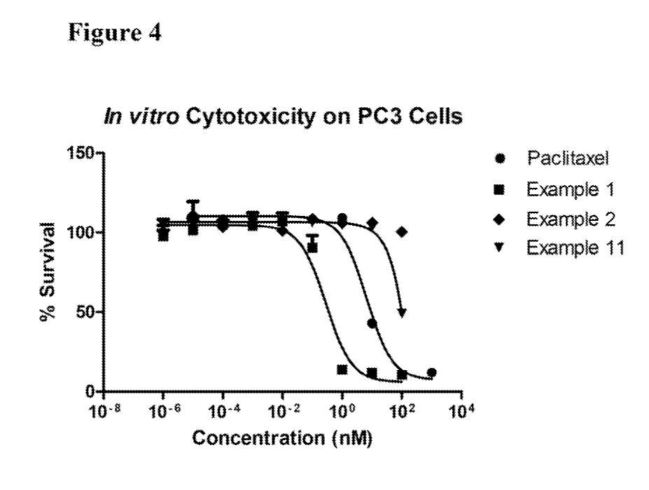

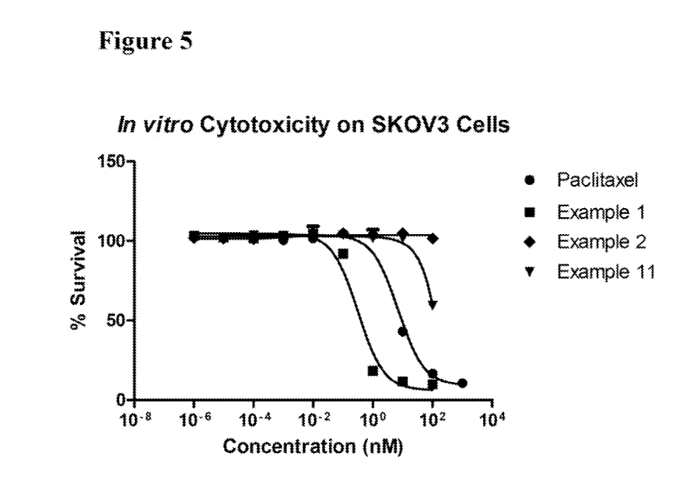

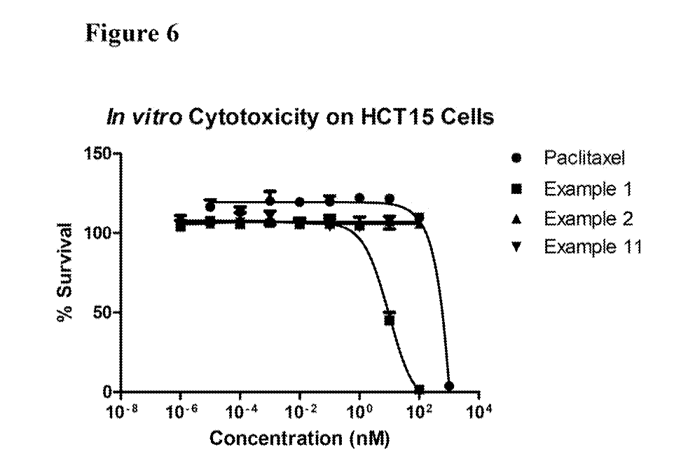

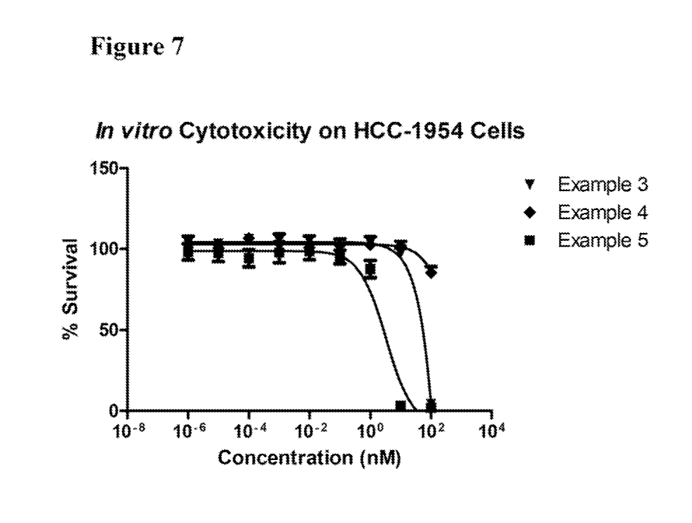

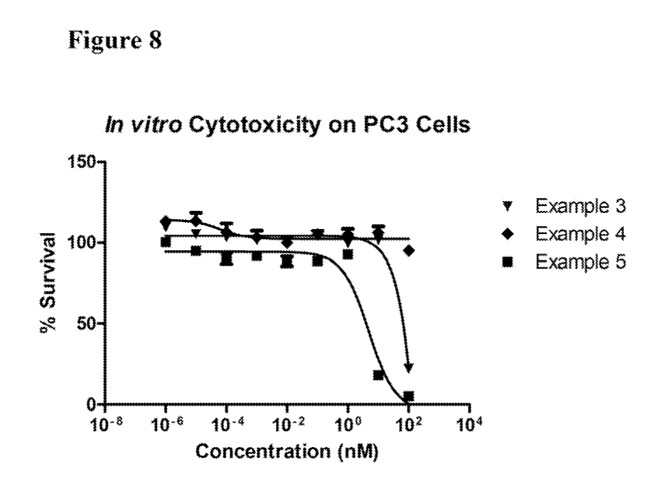

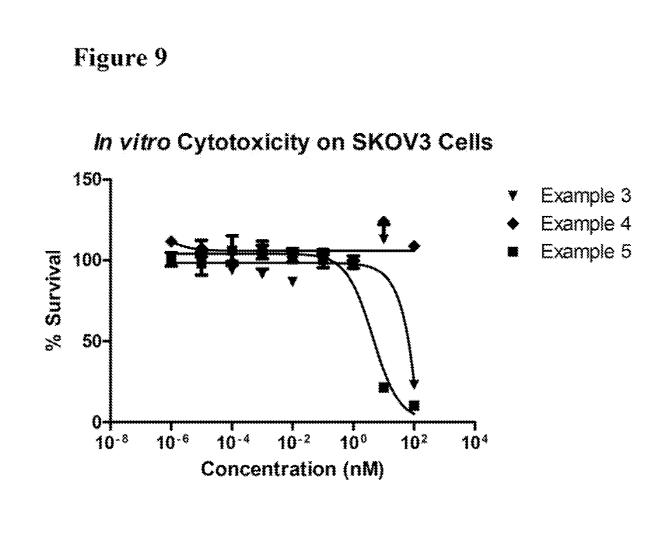

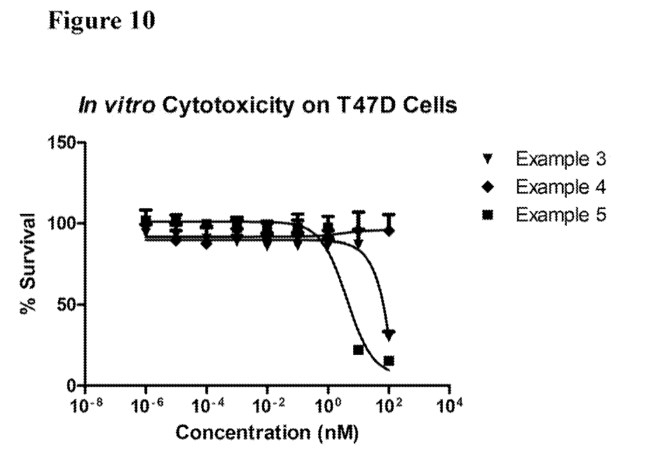

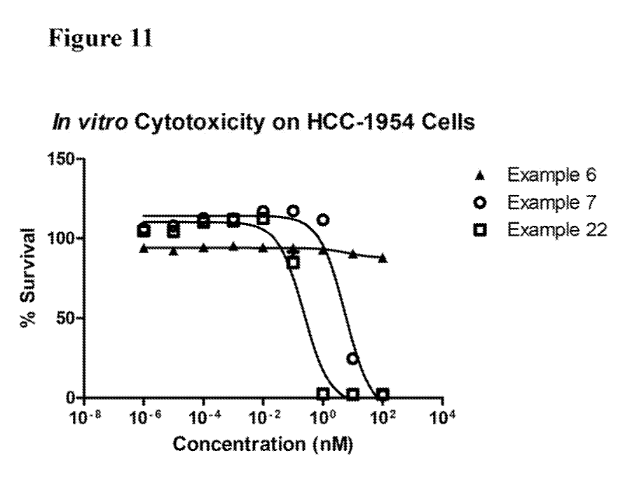

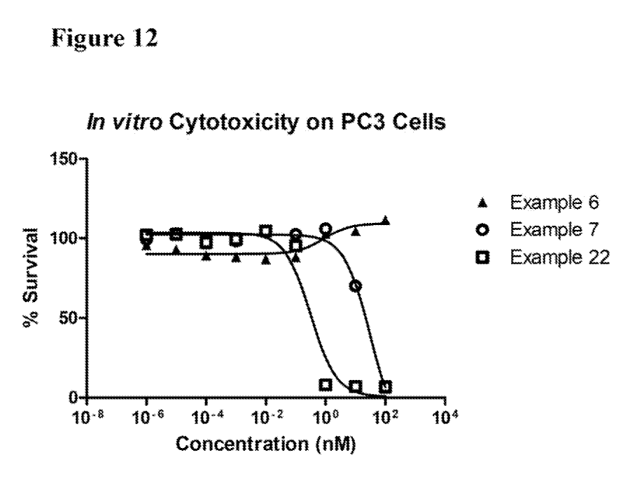

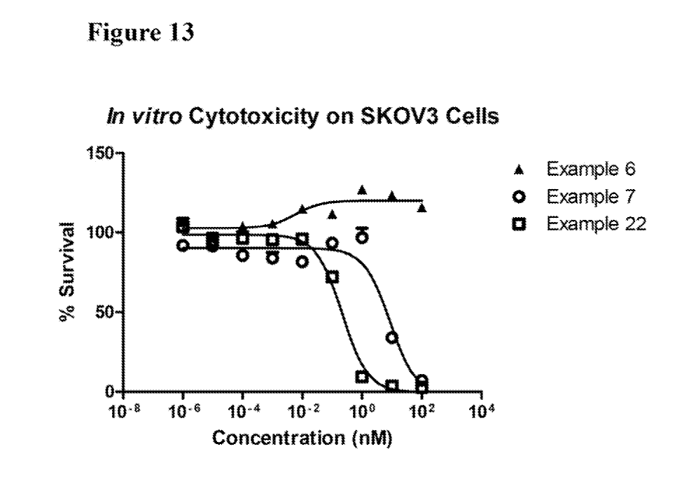

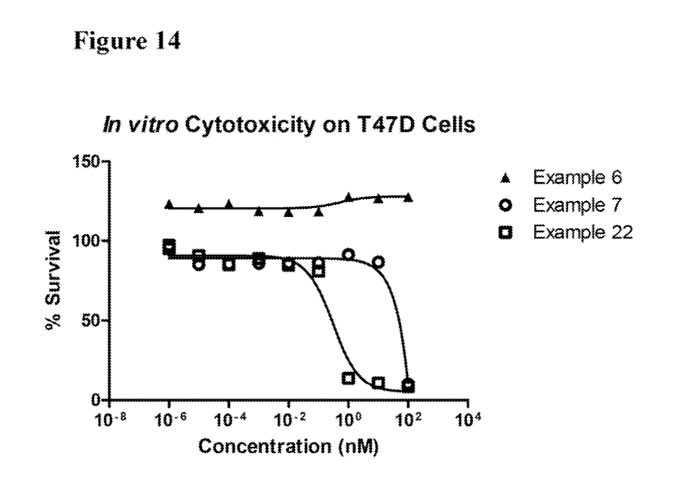

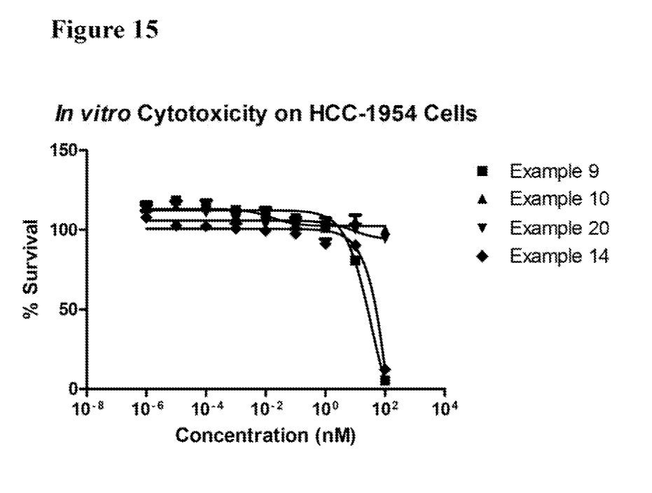

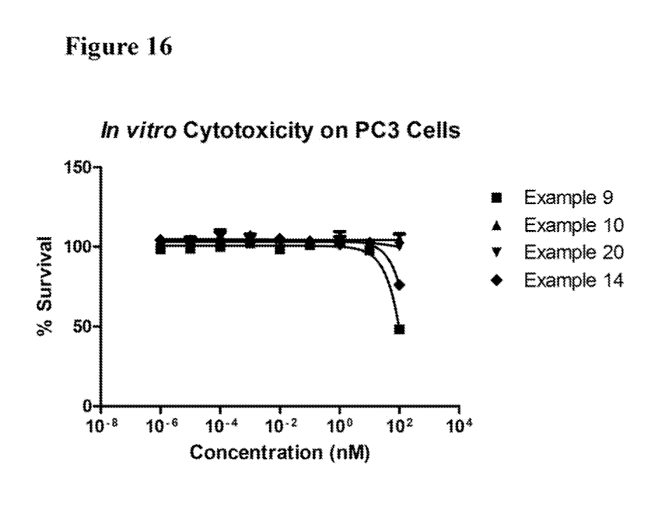

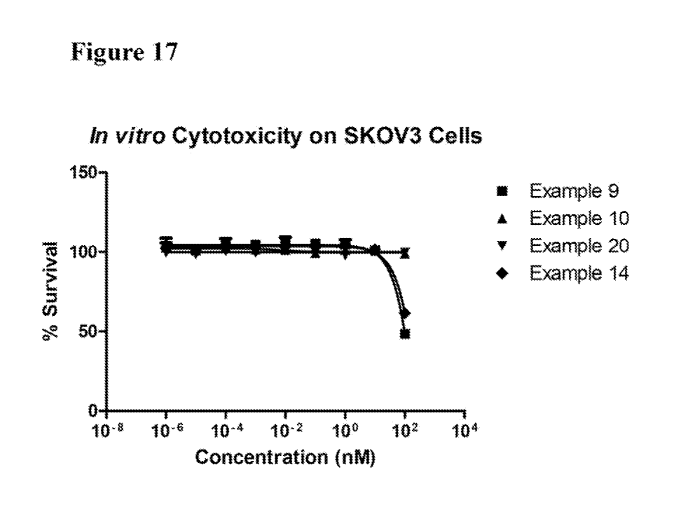

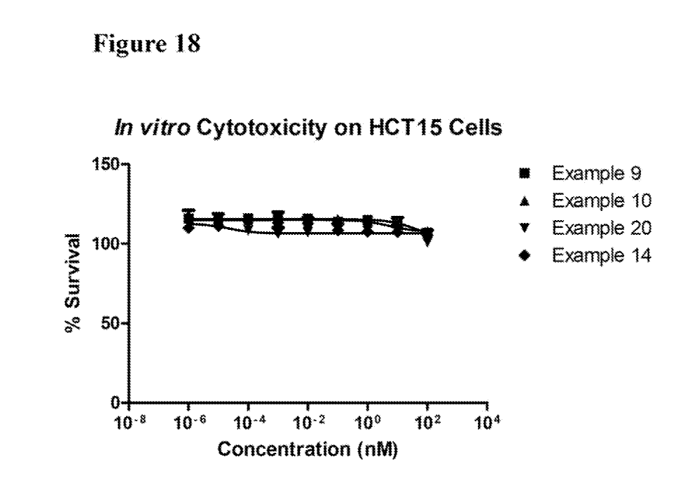

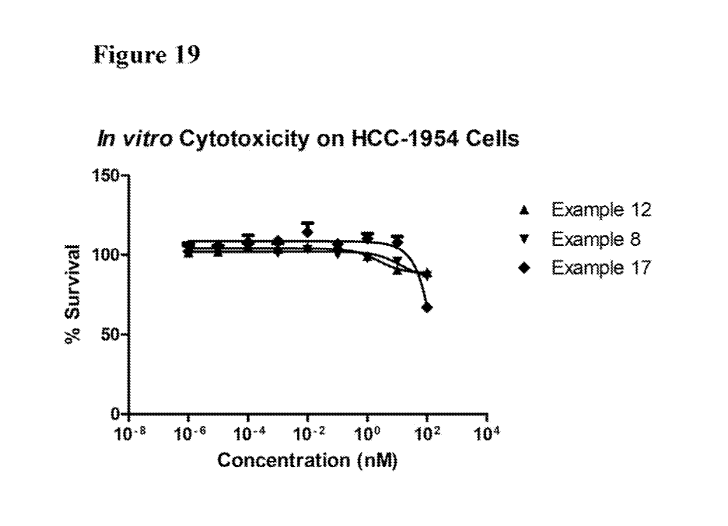

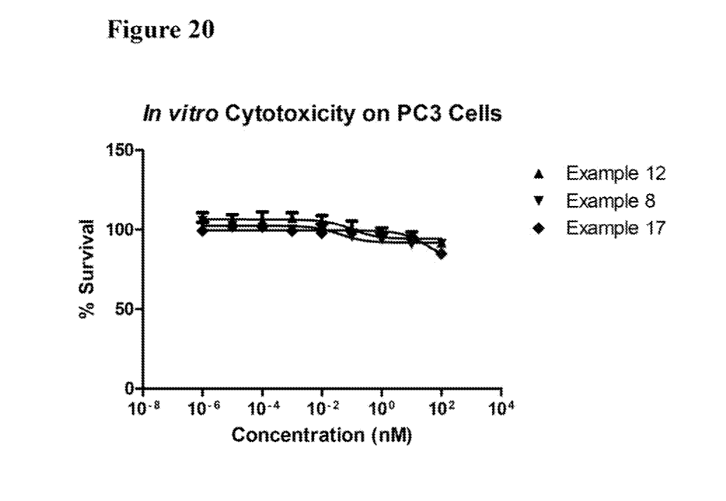

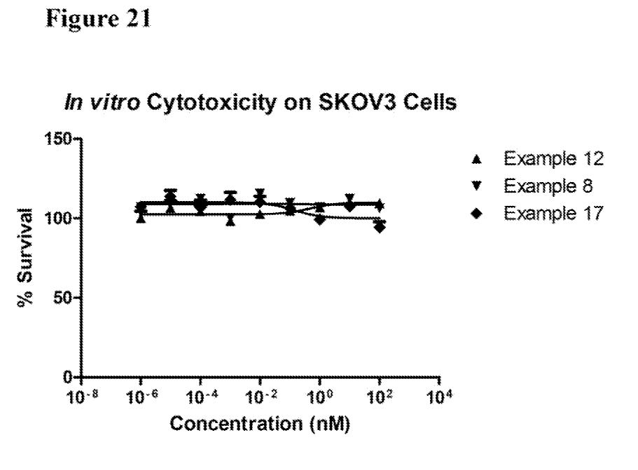

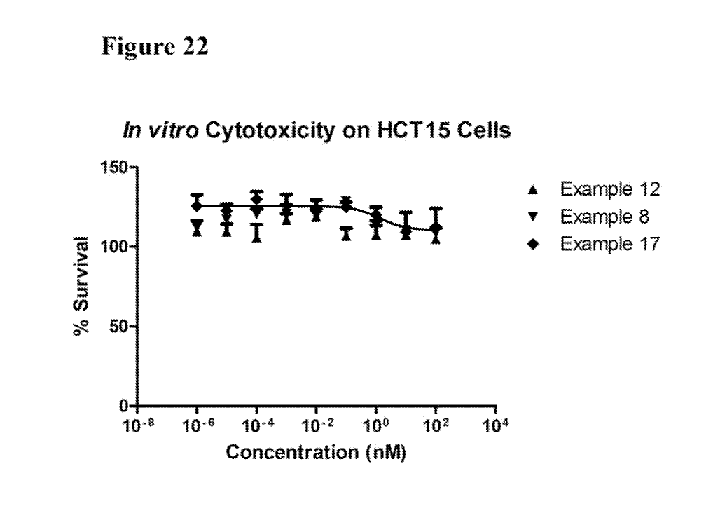

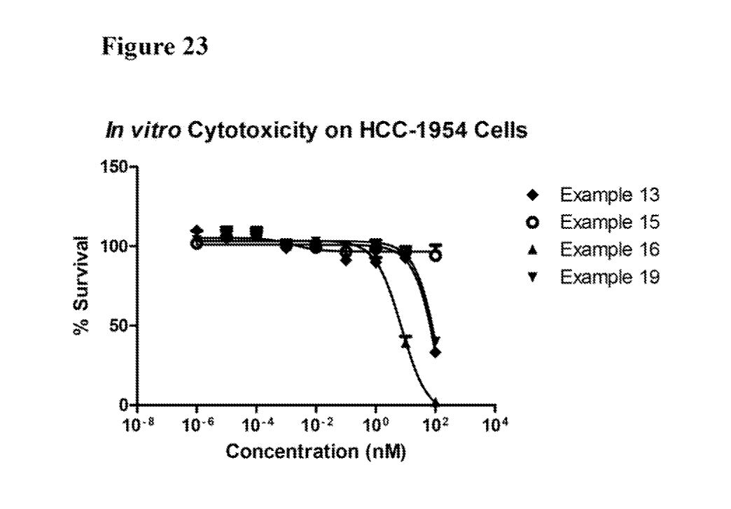

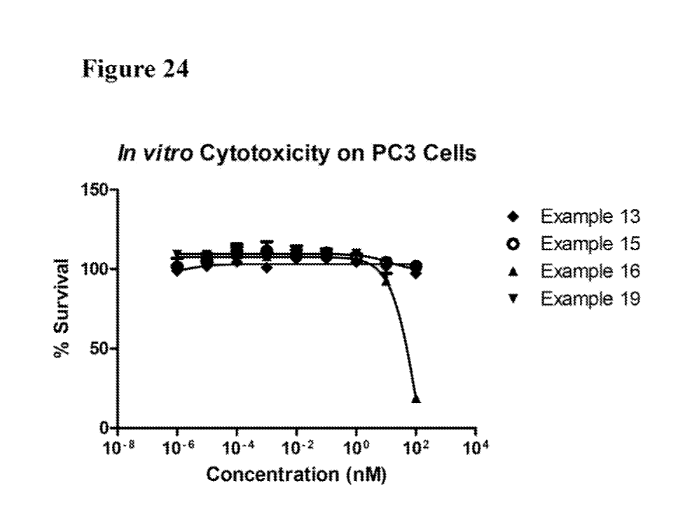

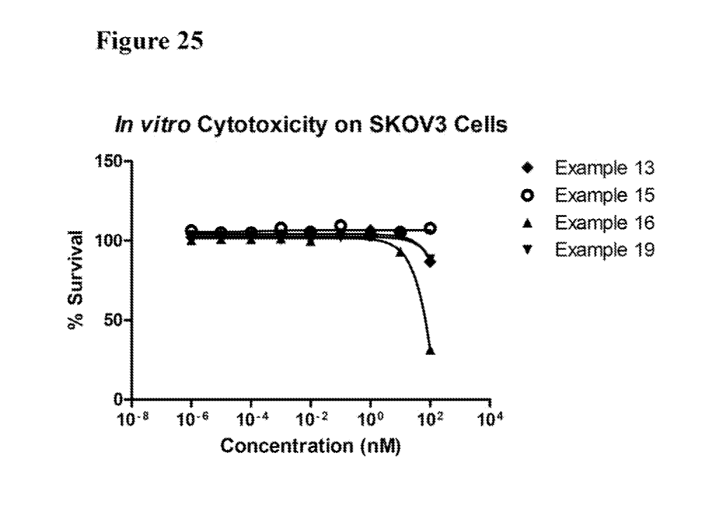

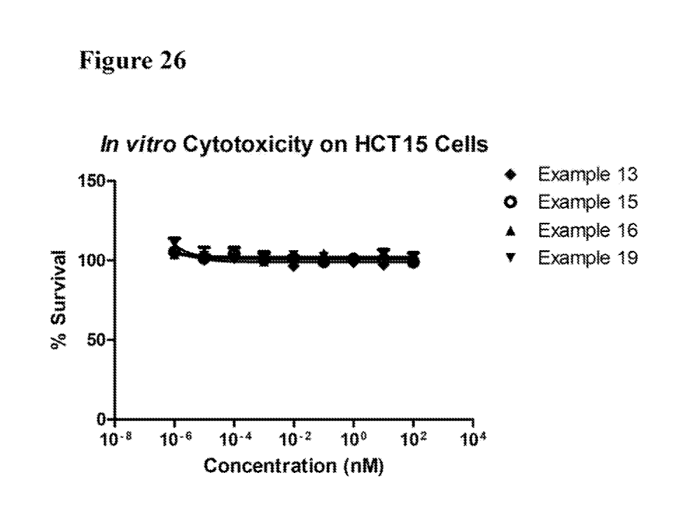

The in vitro efficacy of the compounds of the invention may be measured by evaluating their cytotoxic activity on various cancer cell lines. This assay was conducted in clear tissue-culture treated 96-well plates. The cell lines used were PC3 (human prostate carcinoma), HCC-1954 (human mammary ductal carcinoma), SKOV3 (human ovarian carcinoma), T47D (human mammary adenocarcinoma), SW780 (human bladder transitional cell carcinoma), and HCT15 (human colorectal adenocarcinoma, Pgp-expressing). Cells were seeded at approximately 1,000-3,000 cells per well in 50 μL of growth media (RPMI-1640+10% heat-inactivated fetal bovine serum) and incubated overnight at 37° C. with 5% CO₂ to allow them to attach. Stock solutions of the test compounds in DMSO were diluted with growth media (RPMI-1640+10% heat-inactivated fetal bovine serum). The next day, 50 μL of a 2× stock of vehicle control (DMSO) or compounds at varying concentrations were added to each well in triplicate. In addition, control wells with no cells or untreated cells alone were used. The plates were incubated in the humidified tissue culture incubator with 5% CO₂ at 37° C. for 4 to 6 days after addition of compounds to measure cytotoxicity. After 4 to 6 days, 20 μL of PrestoBlue™ Cell Viability Reagent (Life Technologies, Carlsbad, Calif., Catalog #A13261) was added per well. Plates were incubated at 37° C. for 1 to 2 hours. Fluorescence was recorded at 540ex/590em using a Biotek Synergy™H4 plate reader. Data for compounds tested in this assay are graphed as percent survival compared to untreated control wells, as shown in FIGS. 1-26.

### Example 24

### Determination of Tubulin Polymerization

The inhibition of tubulin polymerization by the compounds of the invention was evaluated on bovine brain tubulin. To evaluate the activity of compounds, tubulin was seeded at approximately 400 g per well in 100 μL of general tubulin buffer, and then treated with 10 μM final concentration of compound in duplicate at the initiation of the assay. Tubulin polymerization assays were usually carried out at 37° C. for 60 minutes after the addition of test compounds. Tubulin polymerization was determined by absorbance spectroscopy using the optical density value at 340 nm. To assess the amount of polymerized tubulin, the optical density value at 340 nm was obtained each minute after the addition of test compounds. For analysis, the extent of tubulin polymerization by the compound-treated tubulin was compared to that of the control, which was buffer-treated tubulin. In particular, tubulin inhibition studies were performed using HTS-Tubulin Polymerization Assay Kit (Cytoskeleton Inc.; Catalog #BK004P), using the following sample protocol:
1. Pre-warm the spectrophotometer and 96-well plates to 37° C. for 30 minutes prior to starting the assay. A warm plate is essential for high polymerization activity and reproducible results.
2. Enter all plate reader parameters (Absorbance at 340 nm, 37° C., one read each minute) so that the spectrophotometer is ready for use. Once the tubulin is aliquoted into the 37° C. wells, the reading must begin immediately.
3. Warm 500 μL of general tubulin buffer to room temperature. Warm buffer is needed for tubulin ligand dilutions.
4. Paclitaxel is included as a control. Use 10 μL of Paclitaxel per well, which brings the final concentration to 10 μM final.
5. Make cold assay buffer: general tubulin buffer, 1 mM GTP, 10% glycerol.
6. Resuspend 4 mgs of tubulin with 1 mL of cold assay buffer to bring the final protein concentration of 4 mg/mL. Place the tubes on ice and allow 3 minutes for the complete resuspension of the protein.
7. Prepare selected compound at 10× concentration in assay buffer.
8. Pipette 10 μL of the 10× concentrated compound into the required number of wells of the pre-warmed plate. Incubate the plate for 2 minutes at 37° C.
9. Pipette 10 μL of assay buffer only into two control wells (buffer-treated tubulin).
10. Pipette 100 μL of tubulin into the required number of wells (two wells should be the zero compound controls, which are buffer-treated).
11. Immediately place the plate into the spectrophotometer at 37° C. and start recording the optical density at 340 nm each minute. Increasing optical density values at 340 nm equate to increasing tubulin polymerization.

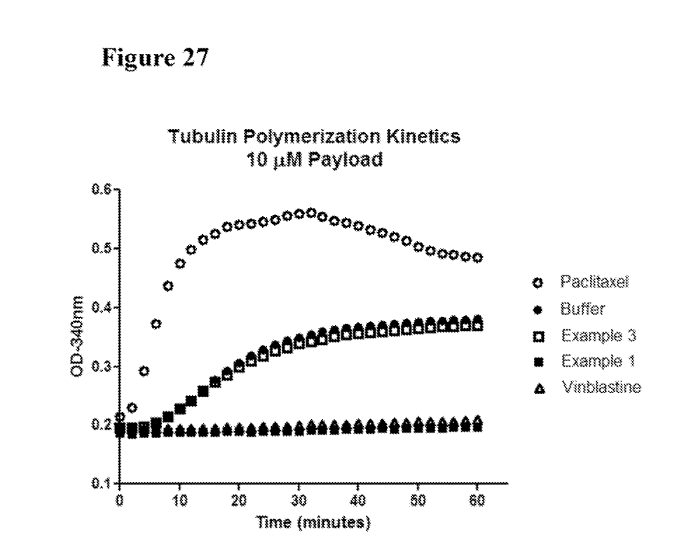

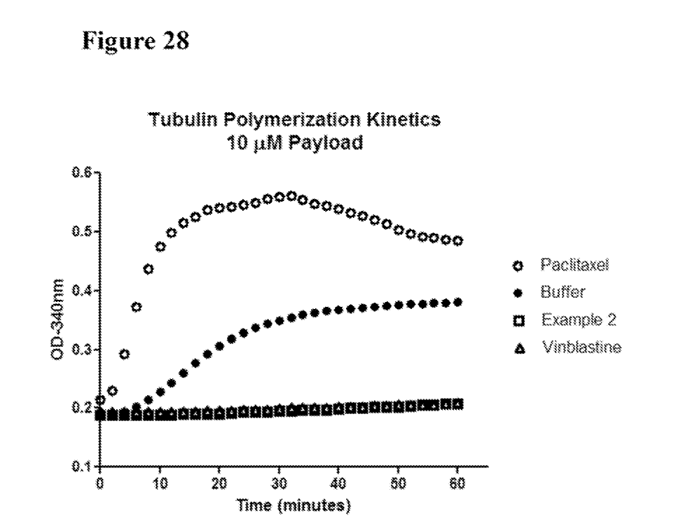

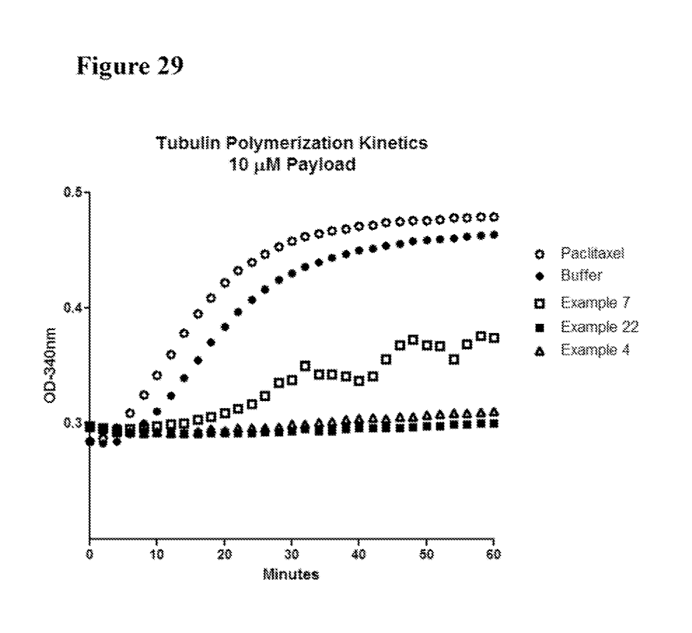

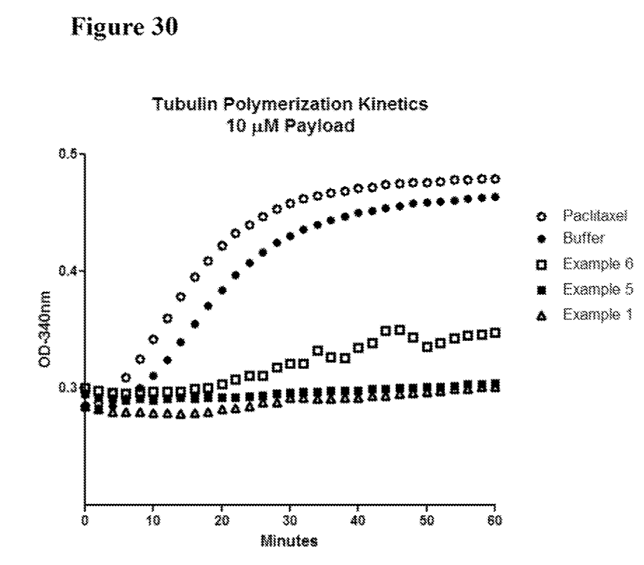

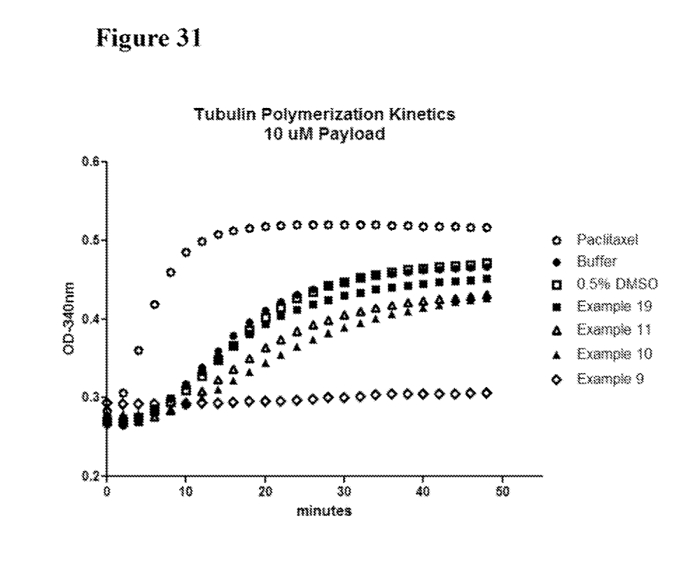

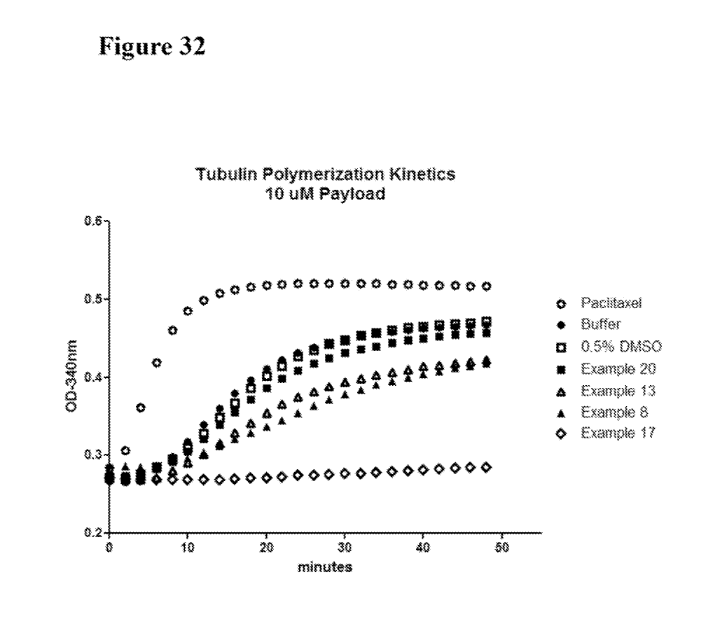

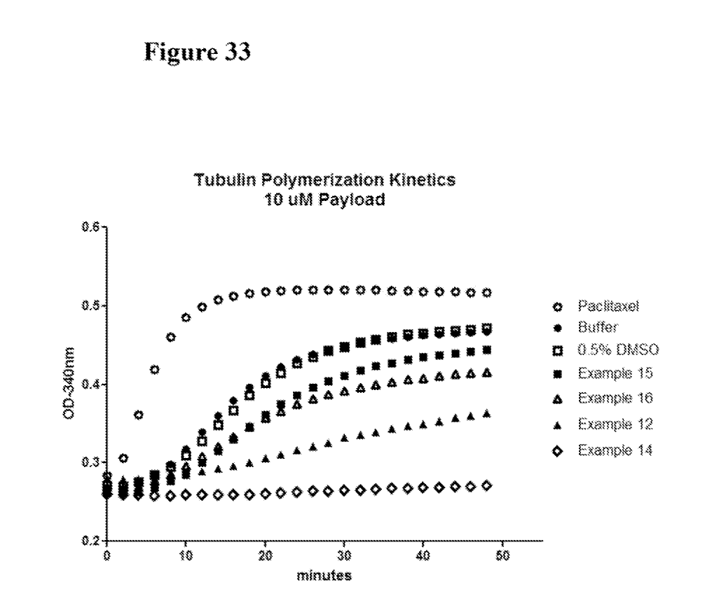

Data for compounds tested in this assay are presented in FIGS. 27-33.

### Example 25

### Determination of In Vivo Efficacy of Test Compounds: Efficacy Evaluation in Subcutaneously Established Human Bladder Cancer Cell Line SW780 Implanted in ICR SCID Mice

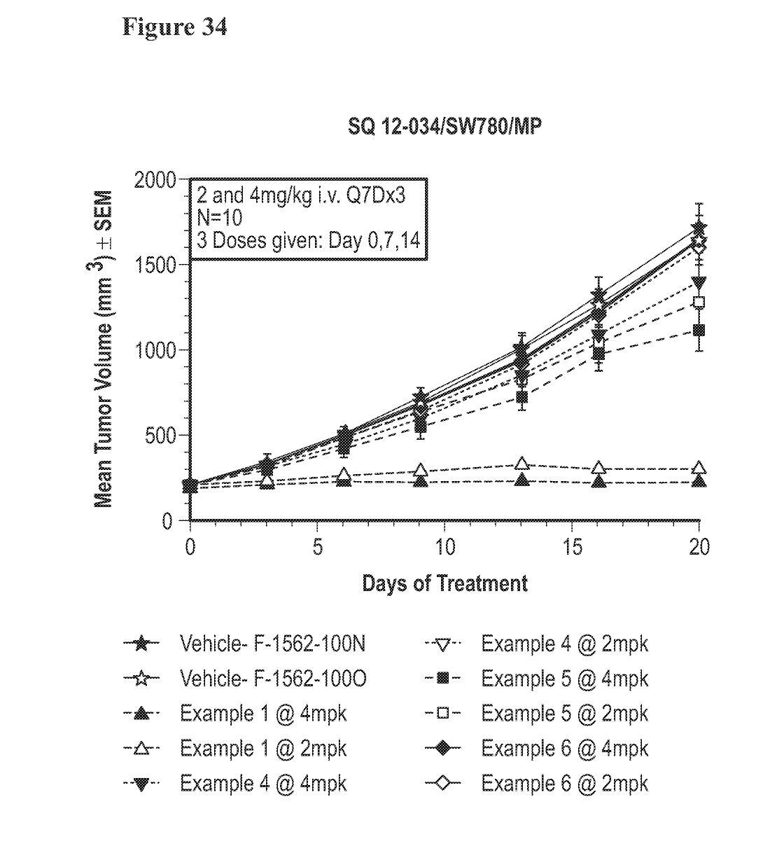

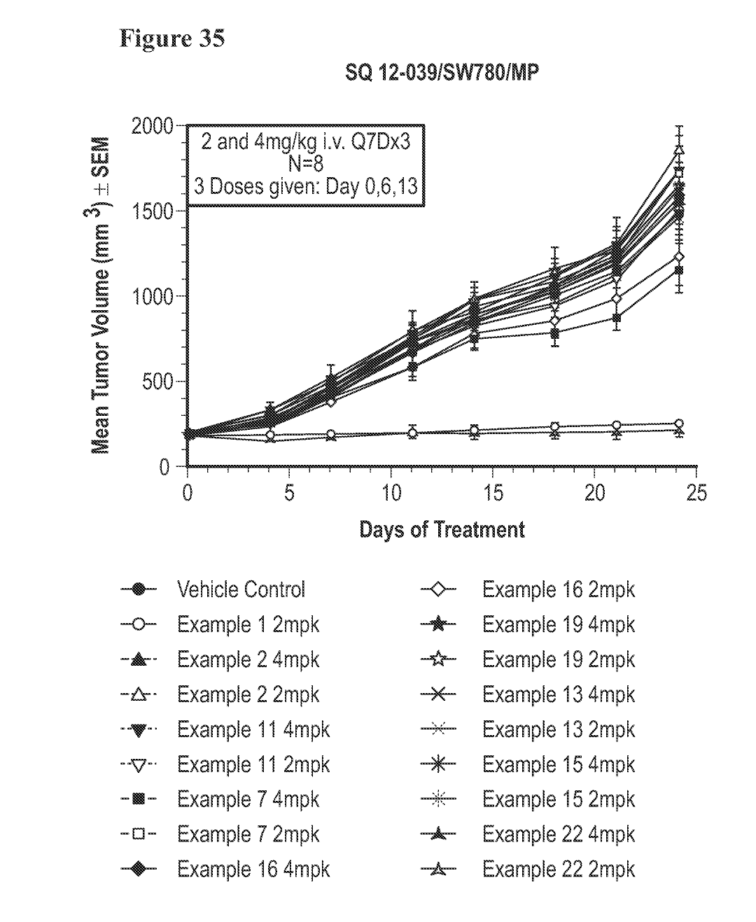

For animal in vivo studies, the test compounds were diluted with 20 mM Histidine, 5% Sucrose, pH 6 with 15% DMSO. Male ICR SCID mice (Taconic Farm, Hudson, N.Y.) were housed in standard rodent micro isolator cages. Environment controls for the animal rooms were set to maintain a temperature between 68-75° F., a relative humidity between 30% to 70%, and an approximate 12 h light/12 h dark cycle. Food and water was provided ad libitum. After 72 h of acclimatization, the mice were implanted with SW780 human bladder cancer cells (2×10⁶ cells/mouse), suspended in 50% complete cultrex (Trevigen, Inc.) mixed with 1×PBS (Gibco), and the tumor growth rate was monitored. When the average tumor volume reached ˜200 mm³, tumors were size-matched and mice were randomized to treatment groups (n=8 or 10). The tumor-bearing mice were treated i.v. with Vehicle or test compound at 2 or 4 mg/kg (mpk) on a QW dosing schedule for 3 weeks. Tumor volume was assessed twice weekly using caliper measurement. Data for compounds tested in this assay are shown in FIGS. 34 and 35. Notably, FIGS. 34 and 35 show that Example 1 caused tumor stasis at both 2 and 4 mg/kg dose levels. FIG. 35 shows that Example 22 showed tumor stasis at 2 mg/kg dose for the entire duration of the study.

While the foregoing written description of the invention enables one of ordinary skill to make and use the invention, those of ordinary skill will understand and appreciate the existence of variations, combinations, and equivalents of the specific embodiment, method, and examples herein. The invention should therefore not be limited by the above-described embodiments, methods, or examples, but rather encompasses all embodiments and methods within the scope and spirit of the invention.

1. A compound of Formula (I): 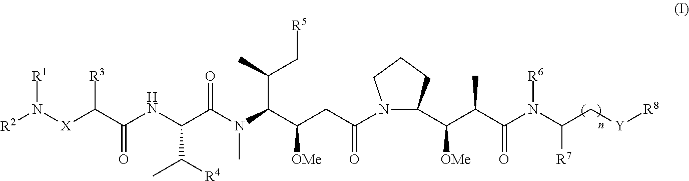
wherein
R¹ and R² are each independently H, methyl, or OH; or R¹ and R² taken together with the N to which they are attached form a 3- to 8-membered heterocyclic ring, optionally substituted with C₁₋₄alkyl, hydroxy, or C₁₋₄alkoxy;
X is O or is absent;
R³ is an amino acid substituent;
wherein R³ is in the R or S stereochemical configuration relative to the remainder of the molecule;
or, alternatively, the —(CH(R³))— group is a phenyl;
R⁴ is methyl or ethyl;
R⁵ is H or methyl;
R⁶ is H or C₁₋₈alkyl;
R⁷ is H, C₁₋₈alkyl, —CO₂R^{a}, —(OCH₂CH₂)ₘ—OR^{b}, —NH—CH₂CH₂—N(CH₃)₂, —B(OR^{c})₂, or tetrazole;
wherein R^{a}, R^{b}, and R^{c} are each independently H or C₁₋₄alkyl;
m is an integer from 1 to 12; and
R⁷ is in the R or S stereochemical configuration relative to the remainder of the molecule;
n is 0, 1, 2, 3, or 4;
Y is O or is absent; and
R⁸ is (1) or (2):
(1) —NR^{d}R^{e} wherein R^{d} and R^{e} are each independently H, C₁₋₄alkyl, OH, SH, —NR^{f}R^{g}, —NHCOR^{g}, halo, —COR^{h}, or a phenyl or 3- to 8-membered heterocyclic ring, each optionally substituted with C₁₋₄alkyl, hydroxy, or C₁₋₄alkoxy;
wherein R^{f} and R^{g} are each independently H, C₁₋₄alkyl, phenyl, or a 3- to 8-membered heterocyclic ring;
R^{h} is H, —OH, —OC₁₋₄alkyl, —NRⁱR^{j}, —NHNH₂, or —ONH₂; and
Rⁱ and R^{j} are each independently H, C₁₋₄alkyl, phenyl, or a 3- to 8-membered heterocyclic ring;
or R^{d} and R^{e} taken together with the nitrogen to which they are attached form a 5- to 8-membered heterocyclic ring, optionally substituted with one or two substituents selected from the group consisting of C₁₋₄alkyl, halo, OH, —OC₁₋₄alkyl, SH, —NR^{k}R^{l}, —NHC(O)R^{l}, —NHCO₂R^{l}, cyano, —CONR^{k}R^{l}, —COR^{m}, phenyl, and a 3- to 8-membered heterocyclic ring;
where R^{k} and R^{l} are each independently H, C₁₋₄alkyl, phenyl, or a 3- to 8-membered heterocyclic ring; or
R^{k} and R^{l} taken together with the nitrogen to which they are attached form a 3- to 8-membered heterocyclic ring; and
R^{m} is H, OH, —OC₁₋₄alkyl, —NHNH₂, or —ONH₂;
(2) a phenyl or a carbon-linked, nitrogen-containing heteroaryl ring, each optionally substituted with one, two, or three substituents selected from the group consisting of halo, OH, SH, —NRⁿR^{o}, —NHCOR^{o}, —COR^{p}, phenyl, anilino, and a 3- to 8-membered heterocyclic ring;
wherein Rⁿ and R^{o} are each independently H, C₁₋₄alkyl, phenyl, or a 3- to 8-membered heterocyclic ring;
R^{p} is H, OH, —NR^{q}R^{r}, —NHNH₂, or —ONH₂; and
Wherein R^{q} and R^{r} are each independently H, C₁₋₄alkyl, phenyl, or a 3- to 8-membered heterocyclic ring;
wherein n is not 0 when R⁸ is —NR^{d}R^{e}; 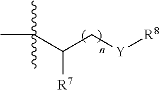
with the proviso that when R¹ and R² are both methyl, R⁷ is not 2-(2-pyridyl)ethyl, phenethyl, 4-hydroxyphenethyl, 2-morpholinoethyl, 3-dimethylaminopropyl, 2-dimethylaminoethyl, 4-aminophenethyl, 2-(5-methoxy-1H-indol-2-yl)ethyl, or 1-methoxy-1-oxo-3-phenylpropan-2-yl;
or a pharmaceutically acceptable salt thereof.

2. The compound according to claim 1, wherein R¹ and R² is are each independently H or methyl.

3. The compound according to claim 1, wherein R³ is (R)-isopropyl.

4. The compound according to claim 1, wherein the group —(CH(R³))— is phenyl.

5. The compound according to claim 1, wherein R⁴ and R⁵ are both methyl.

6. The compound according to claim 1, wherein R⁶ is H.

7. The compound according to claim 1, wherein R⁷ is H.

8. The compound according to claim 1, wherein R^{a}, R^{b}, and R^{c} are each independently H or methyl.

9. The compound according to claim 1, wherein m is 1.

10. The compound according to claim 1, wherein n is 1.

11. The compound according to claim 1, wherein R⁸ is —NR^{d}R^{e}.

12. The compound according to claim 11, wherein R^{d} and R^{e} are each independently H or methyl.

13. The compound according to claim 11, wherein R^{d} and R^{e} taken together with the nitrogen to which they are attached form a 5- to 8-membered heterocyclic ring, optionally substituted with one or two substituents selected from the group consisting of C₁₋₄alkyl, halo, OH, —OC₁₋₄alkyl, SH, —NR^{k}R^{l}, —NHC(O)R^{l}, cyano, —CONR^{k}R^{l}, —COR^{m}, phenyl, and a 3- to 8-membered heterocyclic ring.

14. The compound according to claim 13, wherein R^{d} and R^{e} taken together with the nitrogen to which they are attached form piperazine, N-methylpiperazine, morpholine, maleimido, or 3,5-dioxo-3H-1,2,4-triazol-4(5H)-yl.

15. The compound according to claim 1, wherein R⁸ is an optionally substituted phenyl, pyridine, or imidazole ring.

16. The compound according to claim 1, wherein R⁸ is phenyl, 2-pyridyl, 3-pyridyl, 4-pyridyl, 1-imidazolyl, 2-imidazolyl, or 4-imidazolyl, each optionally substituted.

17. A compound selected from the group consisting of:
(S)—N-((3R,4S,5S)-3-methoxy-1-((S)-2-((1R,2R)-1-methoxy-2-methyl-3-oxo-3-((2-(pyridin-2-yl)ethyl)amino)propyl)pyrrolidin-1-yl)-5-methyl-1-oxoheptan-4-yl)-N,3-dimethyl-2-((S)-3-methyl-2-(methylamino)butanamido)butanamide;
4-amino-N—((S)-1-(((3R,4S,5S)-3-methoxy-1-((S)-2-((1R,2R)-1-methoxy-2-methyl-3-oxo-3-((2-(pyridin-2-yl)ethyl)amino)propyl)pyrrolidin-1-yl)-5-methyl-1-oxoheptan-4-yl)(methyl)amino)-3-methyl-1-oxobutan-2-yl)benzamide;
(S)—N-((3R,4S,5S)-3-methoxy-1-((S)-2-((1R,2R)-1-methoxy-2-methyl-3-oxo-3-((2-(pyridin-3-yl)ethyl)amino)propyl)pyrrolidin-1-yl)-5-methyl-1-oxoheptan-4-yl)-N,3-dimethyl-2-((S)-3-methyl-2-(methylamino)butanamido)butanamide;
4-amino-N—((S)-1-(((3R,4S,5S)-3-methoxy-1-((S)-2-((1R,2R)-1-methoxy-2-methyl-3-oxo-3-((2-(pyridin-3-yl)ethyl)amino)propyl)pyrrolidin-1-yl)-5-methyl-1-oxoheptan-4-yl)(methyl)amino)-3-methyl-1-oxobutan-2-yl)benzamide;
(S)—N-((3R,4S,5S)-3-methoxy-1-((S)-2-((1R,2R)-1-methoxy-2-methyl-3-oxo-3-((2-(pyridin-4-yl)ethyl)amino)propyl)pyrrolidin-1-yl)-5-methyl-1-oxoheptan-4-yl)-N,3-dimethyl-2-((S)-3-methyl-2-(methylamino)butanamido)butanamide;
4-amino-N—((S)-1-(((3R,4S,5S)-3-methoxy-1-((S)-2-((1R,2R)-1-methoxy-2-methyl-3-oxo-3-((2-(pyridin-4-yl)ethyl)amino)propyl)pyrrolidin-1-yl)-5-methyl-1-oxoheptan-4-yl)(methyl)amino)-3-methyl-1-oxobutan-2-yl)benzamide;
(S)—N-((3R,4S,5S)-1-((S)-2-((1R,2R)-3-((2-(1H-imidazol-1-yl)ethyl)amino)-1-methoxy-2-methyl-3-oxopropyl)pyrrolidin-1-yl)-3-methoxy-5-methyl-1-oxoheptan-4-yl)-N,3-dimethyl-2-((S)-3-methyl-2-(methylamino)butanamido)butanamide;
N—((S)-1-(((3R,4S,5S)-1-((S)-2-((1R,2R)-3-((2-(1H-imidazol-1-yl)ethyl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)-4-aminobenzamide;
(S)—N-((3R,4S,5S)-1-((S)-2-((1R,2R)-3-((2-(1H-imidazol-2-yl)ethyl)amino)-1-methoxy-2-methyl-3-oxopropyl)pyrrolidin-1-yl)-3-methoxy-5-methyl-1-oxoheptan-4-yl)-N,3-dimethyl-2-((S)-3-methyl-2-(methylamino)butanamido)butanamide;
N—((S)-1-(((3R,4S,5S)-1-((S)-2-((1R,2R)-3-((2-(1H-imidazol-2-yl)ethyl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)-4-aminobenzamide;
(S)—N-((3R,4S,5S)-1-((S)-2-((1R,2R)-3-((2-(1H-imidazol-4-yl)ethyl)amino)-1-methoxy-2-methyl-3-oxopropyl)pyrrolidin-1-yl)-3-methoxy-5-methyl-1-oxoheptan-4-yl)-N,3-dimethyl-2-((S)-3-methyl-2-(methylamino)butanamido)butanamide;
N—((S)-1-(((3R,4S,5S)-1-((S)-2-((1R,2R)-3-((2-(1H-imidazol-4-yl)ethyl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)-4-aminobenzamide;
(S)—N-((3R,4S,5S)-1-((S)-2-((R,2R)-3-((2-(dimethylamino)ethyl)amino)-1-methoxy-2-methyl-3-oxopropyl)pyrrolidin-1-yl)-3-methoxy-5-methyl-1-oxoheptan-4-yl)-N,3-dimethyl-2-((S)-3-methyl-2-(methylamino)butanamido)butanamide;
(S)-2-((S)-2-(dimethylamino)-3-methylbutanamido)-N-((3R,4S,5S)-3-methoxy-1-((S)-2-((R,2R)-1-methoxy-2-methyl-3-((2-(methylamino)ethyl)amino)-3-oxopropyl)pyrrolidin-1-yl)-5-methyl-1-oxoheptan-4-yl)-N,3-dimethylbutanamide;
(S)—N-((3R,4S,5S)-1-((S)-2-((R,2R)-3-((2-aminoethyl)amino)-1-methoxy-2-methyl-3-oxopropyl)pyrrolidin-1-yl)-3-methoxy-5-methyl-1-oxoheptan-4-yl)-2-((S)-2-(dimethylamino)-3-methylbutanamido)-N,3-dimethylbutanamide;
(S)—N-((3R,4S,5S)-3-methoxy-1-((S)-2-((1R,2R)-1-methoxy-2-methyl-3-((2-morpholinoethyl)amino)-3-oxopropyl)pyrrolidin-1-yl)-5-methyl-1-oxoheptan-4-yl)-N,3-dimethyl-2-((S)-3-methyl-2-(methylamino)butanamido)butanamide;
(S)-2-((S)-2-(dimethylamino)-3-methylbutanamido)-N-((3R,4S,5S)-3-methoxy-1-((S)-2-((1R,2R)-1-methoxy-2-methyl-3-oxo-3-((2-(piperazin-1-yl)ethyl)amino)propyl)pyrrolidin-1-yl)-5-methyl-1-oxoheptan-4-yl)-N,3-dimethylbutanamide;
(S)—N-((3R,4S,5S)-3-methoxy-1-((S)-2-((1R,2R)-1-methoxy-2-methyl-3-oxo-3-((2-(piperazin-1-yl)ethyl)amino)propyl)pyrrolidin-1-yl)-5-methyl-1-oxoheptan-4-yl)-N,3-dimethyl-2-((S)-3-methyl-2-(methylamino)butanamido)butanamide;
(S)—N-((3R,4S,5S)-3-methoxy-1-((S)-2-((1R,2R)-1-methoxy-2-methyl-3-((2-(4-methylpiperazin-1-yl)ethyl)amino)-3-oxopropyl)pyrrolidin-1-yl)-5-methyl-1-oxoheptan-4-yl)-N,3-dimethyl-2-((S)-3-methyl-2-(methylamino)butanamido)butanamide;
4-amino-N—((S)-1-(((3R,4S,5S)-3-methoxy-1-((S)-2-((1R,2R)-1-methoxy-2-methyl-3-((2-morpholinoethyl)amino)-3-oxopropyl)pyrrolidin-1-yl)-5-methyl-1-oxoheptan-4-yl)(methyl)amino)-3-methyl-1-oxobutan-2-yl)benzamide;
4-amino-N—((S)-1-(((3R,4S,5S)-3-methoxy-1-((S)-2-((1R,2R)-1-methoxy-2-methyl-3-oxo-3-((2-(piperazin-1-yl)ethyl)amino)propyl)pyrrolidin-1-yl)-5-methyl-1-oxoheptan-4-yl)(methyl)amino)-3-methyl-1-oxobutan-2-yl)benzamide; and
(S)—N-((3R,4S,5S)-1-((S)-2-((R,2R)-3-((4-aminophenethyl)amino)-1-methoxy-2-methyl-3-oxopropyl)pyrrolidin-1-yl)-3-methoxy-5-methyl-1-oxoheptan-4-yl)-2-((S)-2-(dimethylamino)-3-methylbutanamido)-N,3-dimethylbutanamide;
and pharmaceutically acceptable salts thereof.

18. A pharmaceutical composition comprising an effective amount of at least one compound of Formula (I) as defined in claim 1, or a pharmaceutically acceptable salt thereof.

19. A method of treating a subject suffering from or diagnosed with cancer, comprising administering to a subject in need of such treatment an effective amount of at least one compound of Formula (I) as defined in claim 1, or a pharmaceutically acceptable salt thereof.
